SEQUENCE LISTING (307 sequences)

SEQ ID NO: 1 - DNA, 140 bp, synthetic construct
  source 1..140: mol_type = other DNA
tgcagtggtacaactatatgcccacaataatcaattacaatatgtaactgagattatgcctttgcatcctcgaataccagctgtccagtactggagagaactcagtcttccctagctaattggagctcccaattaagggc

SEQ ID NO: 2 - DNA, 140 bp, synthetic construct
  source 1..140: mol_type = other DNA
ccaacctgacaattctatgacggctgaatcactgggaaataattgaggttaattcctcagtctcctgcacataccacccacctctcctcccagcaggccagggaggatttatgagttaatggtggcattgtcatgtaaag

SEQ ID NO: 3 - DNA, 126 bp, synthetic construct
  source 1..126: mol_type = other DNA
catgctgcaaggggcaaaggtcacaggtgaggcgaaggggagggatggggacaccctttttaacagccagcggtgaaaatttaggctaagacaaggctgaccccactgccgagagagggcgggcag

SEQ ID NO: 4 - DNA, 104 bp, synthetic construct
  source 1..104: mol_type = other DNA
agcagccgtgggcgacctggggaaattaaactcggcggatccggcccattcatcaaaggaaggcactgggacaaagttggccccctccccttctcccccagccc

SEQ ID NO: 5 - DNA, 100 bp, synthetic construct
  source 1..100: mol_type = other DNA
ttcggtgaacacttttgcacaacttacccagctgatcactcgcgccccctcgcttttccatttctctttccccaaccccgtcaccccctgtctcctccgt

SEQ ID NO: 6 - DNA, 372 bp, synthetic construct
  source 1..372: mol_type = other DNA
cgtaaatatataaataaatacgagaacgaaatgaaactttggccccgagcgccagagcgccagagcgcgagagcgcgggagtgtttgtttgtttgaacttcctcgtcgtcgccaccttccttatccccaacctccaccccacctcacccccttccccagcttataatcgcgtttgactgcagccaggggtgggggtgggggtagggagtgtgtgtggaggggagggagaagaggttaaaaaaaagaagacgaagaagacggaaagaaagagatcgcagcaggggtgaagggagcggacgggaagcgatttttgccgactttggattcgtccccggcgtgcgcaagaatggcggcccttcccggcacggtacc

SEQ ID NO: 7 - DNA, 164 bp, synthetic construct
  source 1..164: mol_type = other DNA
gattagaaaagagggaaaaaaatattcgttggcgatttcttttcgttattggtcctccgctcccttgcgccctggcccccttccctcctcatgaataaaagaaaagtccgaacctatcagatcgcgctcactgcctcctcctcctcctacctcctcctcctcct

SEQ ID NO: 8 - DNA, 66 bp, synthetic construct
  source 1..66: mol_type = other DNA
gatcaagtgcgcgccggcagccacgcaggcgggagagcgcgcaaacccggggatttgcagtggcac

SEQ ID NO: 9 - DNA, 81 bp, synthetic construct
  source 1..81: mol_type = other DNA
ctctgtggcgtgcaattacaaaattgggagtggaggccaagttcccagatgcccagggtcggaggatcgcagtgggggaga

SEQ ID NO: 10 - DNA, 79 bp, synthetic construct
  source 1..79: mol_type = other DNA
gggcaaaggggaggcgggggccaaaggaaagggtgtggggaggggatgcgaagggagggggtcggctgcacttggaact

SEQ ID NO: 11 - DNA, 103 bp, synthetic construct
  source 1..103: mol_type = other DNA
gcaaaggcagggctggagacgccttcagaaatggaaatgggacccatttgtttataatttactctctgacacttaacaggaacctgatgggaccaataaaggc

SEQ ID NO: 12 - DNA, 57 bp, synthetic construct
  source 1..57: mol_type = other DNA
gaattcatgttccctcaacacctttgcagctgtctcttccacactcatttcccatct

SEQ ID NO: 13 - DNA, 115 bp, synthetic construct
  source 1..115: mol_type = other DNA
ttcctcccgcctgcccctcctcccctcagctcggccaattaggcccctgacacctggagccgctgacaaactgtttctgtcacttgctctctgtctttcattagggccgcagaag

SEQ ID NO: 14 - DNA, 139 bp, synthetic construct
  source 1..139: mol_type = other DNA
tgtgtcccaggcactgggaataagacgccaagtctgtccccaggcagctcctacacaaatccaggcacgtcccagccaaatccaggcacgtcatggccacaagaaaacaacaaacagggtgaggctcagtctgttccca

SEQ ID NO: 15 - DNA, 500 bp, synthetic construct
  source 1..500: mol_type = other DNA
cggtgaacacttttgcacaacttacccagctgatcactcgcgccccctcgcttttccatttctctttccccaaccccgtcaccccctgtctcctccgtccagccctgaaacccgaggaggctccttcttccgtctgtccccgggtctcctaggggacggggctgtgaaagctggtgtggagggagaagcgagtgtggtccggagaaagaaggcgtggagaagagggagggagcgagagcgagagaataaatatataaataaatacgagaacgaaatccactccgcagtctccgggctcggaaactttggccccgagcgccagagcgccagagcgcgagagcgcggcgctcgccactctgaggctggcggcctcgattccggccgcgttcccccggcccccctccgccgcggggcctggtctccgggttctgccaggcgcatcagcccgcacaacttctggccgaggccagccggcagaggcggacttggggttggagtgt

SEQ ID NO: 16 - DNA, 847 bp, synthetic construct
  source 1..847: mol_type = other DNA
gactccgagagcagccgtgggcgacctgggggccggggactcggcggatccggcccattcatcaaaggaaggcactgggacaaagttggccccctccccttctcccccagcccccaactcgccgggatcggctccctctcttttcacggcggttcaaattctcccccaaaacgaggtcgagccggggattagaaaagagggaaaaaaatattcgttggcgatttcttttcgttattggtcctccgctcccttgcgccctggcccccttccctcctcatgaataaaagaaaagtccgaacctatcagatcgcgctcactgcctcctcctcctcctcctcctcgtacttcggtgaacacttttgcacaacttacccagctgatcactcgcgccccctcgcttttccatttctctttccccaaccccgtcaccccctgtctcctccgtccagccctgaaacccgaggaggctccttcttccgtctgtccccgggtctcctaggggacggggctgtgaaagctggtgtggagggagaagcgagtgtggtccggagaaagaaggcgtggagaagagggagggagcgagagcgagagaataaatatataaataaatacgagaacgaaatccactccgcagtctccgggctcggaaactttggccccgagcgccagagcgccagagcgcgagagcgcggcgctcgccactctgaggctggcggcctcgattctggccgcgttcccccggcccccctccgccgcggggcctggtctccgggttctgccaggcgcatcagcccgcacaacttctggccgaggccagccggcagaggcggacttggggttggagtgt

SEQ ID NO: 17 - DNA, 251 bp, synthetic construct
  source 1..251: mol_type = other DNA
gttttatctaaccttatagacgagcccccaggcctttgcacctgctgttccctctgcctggaaggacactcacttcccttttcctagagctcaggagccacctccactgggaagccttccctgcagcaacacactccacaccatccttccttttccttgcagaaccttggatcacacctgctccggctattctgatgtcatctccatctcctggagacagttgctgcatgagaacaaaggcagggcccaga

SEQ ID NO: 18 - DNA, 197 bp, synthetic construct
  source 1..197: mol_type = other DNA
agcagctgggggcatttatgggccttcctataaacttctcctccagcagctggtcacaaagctggttaatctcccagagtgctcagcttaaaacccgtgactcacagcacagccagtgtgggggagggggtggctgcctccaatacgtggcgcccagagtcagctgttctggggccttctctggtttctccaactga

SEQ ID NO: 19 - DNA, 67 bp, synthetic construct
  source 1..67: mol_type = other DNA
ctggggcagctgctgggggctgcagctgcttaatcccagtccttggtgccccccaggctggcaggtg

SEQ ID NO: 20 - DNA, 113 bp, synthetic construct
  source 1..113: mol_type = other DNA
tggggcaggtgcaggctggattgggtttccagaggctatatatataaaggctgccggggggcccagccaggcccacattcctttccagaggccagctctccatttatagcccc

SEQ ID NO: 21 - DNA, 295 bp, synthetic construct
  source 1..295: mol_type = other DNA
tccccctcccccagctgcccgcctgggctccggggcgtttaggctactacggataaatagcccagggcgcctggcgagaagctaggggtgaggaagccctggggcgctgccgccgctttccttaaccacaaatcaggccggacaggagagggaggggtgggggacagtgggtgggcattcagactgccagcactttgctatctacagccggggctcccgagcggcagaaagttccggccactctctgccgcttgggttgggcgaagccaggaccgtgccgcgccaccgccaggat

SEQ ID NO: 22 - DNA, 169 bp, synthetic construct
  source 1..169: mol_type = other DNA
gtaaggccgggaccccaggaagtgaggaagttagggcggcgctcgggatatcagggacgcgtttccgagggcggggagctggccttgcgggaggtttgggccaggatccttcccgagagagaggacccccttgtcctgggcagctgtcactggggtagcctgttttgga

SEQ ID NO: 23 - skipped

SEQ ID NO: 24 - DNA, 1859 bp, synthetic construct
  source 1..1859: mol_type = other DNA
tctagatgcagtggtacaactatatgcccacaataatcaattacaatatgtaactgagattatgcctttgcatcctcgaataccagctgtccagtactggagagaactcagtcttccctagctaattggagctcccaattaagggcagatctccaacctgacaattctatgacggctgaatcactgggaaataattgaggttaattcctcagtctcctgcacataccacccacctctcctcccagcaggccagggaggatttatgagttaatggtggcattgtcatgtaaagcgctcatgctgcaaggggcaaaggtcacaggtgaggcgaaggggagggatggggacaccctttttaacagccagcggtgaaaatttaggctaagacaaggctgaccccactgccgagagagggcgggcagaaaactcgagcagccgtgggcgacctggggaaattaaactcggcggatccggcccattcatcaaaggaaggcactgggacaaagttggccccctccccttctcccccagcccttcggtgaacacttttgcacaacttacccagctgatcactcgcgccccctcgcttttccatttctctttccccaaccccgtcaccccctgtctcctccgtgagagcgtaaatatataaataaatacgagaacgaaatgaaactttggccccgagcgccagagcgccagagcgcgagagcgcgggagtgtttgtttgtttgaacttcctcgtcgtcgccaccttccttatccccaacctccaccccacctcacccccttccccagcttataatcgcgtttgactgcagccaggggtgggggtgggggtagggagtgtgtgtggaggggagggagaagaggttaaaaaaaagaagacgaagaagacggaaagaaagagatcgcagcaggggtgaagggagcggacgggaagcgatttttgccgactttggattcgtccccggcgtgcgcaagaatggcggcccttcccggcacggtacccatatgattagaaaagagggaaaaaaatattcgttggcgatttcttttcgttattggtcctccgctcccttgcgccctggcccccttccctcctcatgaataaaagaaaagtccgaacctatcagatcgcgctcactgcctcctcctcctcctacctcctcctcctcctgtttaaacgatcaagtgcgcgccggcagccacgcaggcgggagagcgcgcaaacccggggatttgcagtggcacgtgctctgtggcgtgcaattacaaaattgggagtggaggccaagttcccagatgcccagggtcggaggatcgcagtgggggagactagtgggcaaaggggaggcgggggccaaaggaaagggtgtggggaggggatgcgaagggagggggtcggctgcacttggaactaggcctgcaaaggcagggctggagacgccttcagaaatggaaatgggacccatttgtttataatttactctctgacacttaacaggaacctgatgggaccaataaaggcgttaacgaattcatgttccctcaacacctttgcagctgtctcttccacactcatttcccatctacgcgtttcctcccgcctgcccctcctcccctcagctcggccaattaggcccctgacacctggagccgctgacaaactgtttctgtcacttgctctctgtctttcattagggccgcagaagctagctgtgtcccaggcactgggaataagacgccaagtctgtccccaggcagctcctacacaaatccaggcacgtcccagccaaatccaggcacgtcatggccacaagaaaacaacaaacagggtgaggctcagtctgttccca

SEQ ID NO: 25 - DNA, 1257 bp, synthetic construct
  source 1..1257: mol_type = other DNA
tctagatgcagtggtacaactatatgcccacaataatcaattacaatatgtaactgagattatgcctttgcatcctcgaataccagctgtccagtactggagagaactcagtcttccctagctaattggagctcccaattaagggcagatctccaacctgacaattctatgacggctgaatcactgggaaataattgaggttaattcctcagtctcctgcacataccacccacctctcctcccagcaggccagggaggatttatgagttaatggtggcattgtcatgtaaagcgctcatgctgcaaggggcaaaggtcacaggtgaggcgaaggggagggatggggacaccctttttaacagccagcggtgaaaatttaggctaagacaaggctgaccccactgccgagagagggcgggcagaaaactcgagcagccgtgggcgacctggggaaattaaactcggcggatccggcccattcatcaaaggaaggcactgggacaaagttggccccctccccttctcccccagcccttcggtgaacacttttgcacaacttacccagctgatcactcgcgccccctcgcttttccatttctctttccccaaccccgtcaccccctgtctcctccgtgagagcgtaaatatataaataaatacgagaacgaaatgaaactttggccccgagcgccagagcgccagagcgcgagagcgcgggagtgtttgtttgtttgaacttcctcgtcgtcgccaccttccttatccccaacctccaccccacctcacccccttccccagcttataatcgcgtttgactgcagccaggggtgggggtgggggtagggagtgtgtgtggaggggagggagaagaggttaaaaaaaagaagacgaagaagacggaaagaaagagatcgcagcaggggtgaagggagcggacgggaagcgatttttgccgactttggattcgtccccggcgtgcgcaagaatggcggcccttcccggcacggtacccatatgattagaaaagagggaaaaaaatattcgttggcgatttcttttcgttattggtcctccgctcccttgcgccctggcccccttccctcctcatgaataaaagaaaagtccgaacctatcagatcgcgctcactgcctcctcctcctcctacctcctcctcctcctgtttaaacgatcaagtgcgcgccggcagccacgcaggcgggagagcgcgcaaacccggggatttgcagtggcacgtg

SEQ ID NO: 26 - DNA, 830 bp, synthetic construct
  source 1..830: mol_type = other DNA
ctcgagcagccgtgggcgacctggggaaattaaactcggcggatccggcccattcatcaaaggaaggcactgggacaaagttggccccctccccttctcccccagcccttcggtgaacacttttgcacaacttacccagctgatcactcgcgccccctcgcttttccatttctctttccccaaccccgtcaccccctgtctcctccgtgagagcgtaaatatataaataaatacgagaacgaaatgaaactttggccccgagcgccagagcgccagagcgcgagagcgcgggagtgtttgtttgtttgaacttcctcgtcgtcgccaccttccttatccccaacctccaccccacctcacccccttccccagcttataatcgcgtttgactgcagccaggggtgggggtgggggtagggagtgtgtgtggaggggagggagaagaggttaaaaaaagaagacgaagaagacggaaagaaagagatcgcagcaggggtgaagggagcggacgggaagcgatttttgccgactttggattcgtccccggcgtgcgcaagaatggcggcccttcccggcacggtacccatatgattagaaaagagggaaaaaaatattcgttggcgatttcttttcgttattggtcctccgctcccttgcgccctggcccccttccctcctcatgaataaaagaaaagtccgaacctatcagatcgcgctcactgcctcctcctcctcctacctcctcctcctcctgtttaaacgatcaagtgcgcgccggcagccacgcaggcgggagagcgcgcaaacccggggatttgcagtggcacgtg

SEQ ID NO: 27 - DNA, 585 bp, synthetic construct
  source 1..585: mol_type = other DNA
ctcgagcagccgtgggcgacctggggaaattaaactcggcggatccggcccattcatcaaaggaaggcactgggacaaagttggccccctccccttctcccccagcccttcggtgaacacttttgcacaacttacccagctgatcactcgcgccccctcgcttttccatttctctttccccaaccccgtcaccccctgtctcctccgtgagagcgtaaatatataaataaatacgagaacgaaatgaaactttggccccgagcgccagagcgccagagcgcgagagcgcgggagtgtttgtttgtttgaacttcctcgtcgtcgccaccttccttatccccaacctccaccccacctcacccccttccccagcttataatcgcgtttgactgcagccaggggtgggggtgggggtagggagtgtgtgtggaggggagggagaagaggttaaaaaaaagaagacgaagaagacggaaagaaagagatcgcagcaggggtgaagggagcggacgggaagcgatttttgccgactttggattcgtccccggcgtgcgcaagaatggcggcccttcccggcacggtacc

SEQ ID NO: 28 - DNA, 500 bp, synthetic construct; identical to SEQ ID NO: 15
  source 1..500: mol_type = other DNA

SEQ ID NO: 29 - DNA, 847 bp, synthetic construct; identical to SEQ ID NO: 16
  source 1..847: mol_type = other DNA

SEQ ID NO: 30 - DNA, 683 bp, synthetic construct
  source 1..683: mol_type = other DNA
agcagctgggggcatttatgggccttcctataaacttctcctccagcagctggtcacaaagctggttaatctcccagagtgctcagcttaaaacccgtgactcacagcacagccagtgtgggggagggggtggctgcctccaatacgtggcgcccagagtcagctgttctggggccttctctggtttctccaactgactggggcagctgctgggggctgcagctgcttaatcccagtccttggtgccccccaggctggcaggtgaagctttggggcaggtgcaggctggattgggtttccagaggctatatatataaaggctgccggggggcccagccaggcccacattcctttccagaggccagctctccatttatagccccactagtccccctcccccagctgcccgcctgggctccggggcgtttaggctactacggataaatagcccagggcgcctggcgagaagctaggggtgaggaagccctggggcgctgccgccgctttccttaaccacaaatcaggccggacaggagagggaggggtgggggacagtgggtgggcattcagactgccagcactttgctatctacagccggggctcccgagcggcagaaagttccggccactctctgccgcttgggttgggcgaagccaggaccgtgccgcgccaccgccaggat

SEQ ID NO: 31 - DNA, 625 bp, synthetic construct
  source 1..625: mol_type = other DNA
agcagctgggggcatttatgggccttcctataaacttctcctccagcagctggtcacaaagctggttaatctcccagagtgctcagcttaaaacccgtgactcacagcacagccagtgtgggggagggggtggctgcctccaatacgtggcgcccagagtcagctgttctggggccttctctggtttctccaactggtcgacaatctaagcttggggcaggtgcaggctggattgggtttccagaggctatatatataaaggctgccggggggcccagccaggcccacattcctttccagaggccagctctccatttatagccccactagtccccctcccccagctgcccgcctgggctccggggcgtttaggctactacggataaatagcccagggcgcctggcgagaagctaggggtgaggaagccctggggcgctgccgccgctttccttaaccacaaatcaggccggacaggagagggaggggtgggggacagtgggtgggcattcagactgccagcactttgctatctacagccggggctcccgagcggcagaaagttccggccactctctgccgcttgggttgggcgaagccaggaccgtgccgcgccaccgccaggat

SEQ ID NO: 32 - DNA, 588 bp, synthetic construct
  source 1..588: mol_type = other DNA
agcagctgggggcatttatgggccttcctataaacttctcctccagcagctggtcacaaagctggttaatctcccagagtgctcagcttaaaacccgtgactcacagcacagccagtgtgggggagggggtggctgcctccaatacgtggcgcccagagtcagctgttctggggccttctctggtttctccaactgaattctggggcagctgctgggggctgcagctgcttaatcccagtccttggtgccccccaggctggcaggtgaagcttggcagctgttacatcactagtccccctcccccagctgcccgcctgggctccggggcgtttaggctactacggataaatagcccagggcgcctggcgagaagctaggggtgaggaagccctggggcgctgccgccgctttccttaaccacaaatcaggccggacaggagagggaggggtgggggacagtgggtgggcattcagactgccagcactttgctatctacagccggggctcccgagcggcagaaagttccggccactctctgccgcttgggttgggcgaagccaggaccgtgccgcgccaccgccaggat

SEQ ID NO: 33 - DNA, 515 bp, synthetic construct
  source 1..515: mol_type = other DNA
agcagctgggggcatttatgggccttcctataaacttctcctccagcagctggtcacaaagctggttaatctcccagagtgctcagcttaaaacccgtgactcacagcacagccagtgtgggggagggggtggctgcctccaatacgtggcgcccagagtcagctgttctggggccttctctggtttctccaactggtcgacggcagctgttaggactagtccccctcccccagctgcccgcctgggctccggggcgtttaggctactacggataaatagcccagggcgcctggcgagaagctaggggtgaggaagccctggggcgctgccgccgctttccttaaccacaaatcaggccggacaggagagggaggggtgggggacagtgggtgggcattcagactgccagcactttgctatctacagccggggctcccgagcggcagaaagttccggccactctctgccgcttgggttgggcgaagccaggaccgtgccgcgccaccgccaggat

SEQ ID NO: 34 - DNA, 412 bp, synthetic construct
  source 1..412: mol_type = other DNA
ggggcaggtgcaggctggattgggtttccagaggctatatatataaaggctgccggggggcccagccaggcccacattcctttccagaggccagctctccatttatagccccactagtccccctcccccagctgcccgcctgggctccggggcgtttaggctactacggataaatagcccagggcgcctggcgagaagctaggggtgaggaagccctggggcgctgccgccgctttccttaaccacaaatcaggccggacaggagagggaggggtgggggacagtgggtgggcattcagactgccagcactttgctatctacagccggggctcccgagcggcagaaagttccggccactctctgccgcttgggttgggcgaagccaggaccgtgccgcgccaccgccaggat

SEQ ID NO: 35 - DNA, 703 bp, synthetic construct
  source 1..703: mol_type = other DNA
agcagctgggggcatttatgggccttcctataaacttctcctccagcagctggtcacaaagctggttaatctcccagagtgctcagcttaaaacccgtgactcacagcacagccagtgtgggggagggggtggctgcctccaatacgtggcgcccagagtcagctgttctggggccttctctggtttctccaactgagctagacggtgaacacttttgcacaacttacccagctgatcactcgcgccccctcgcttttccatttctctttccccaaccccgtcaccccctgtctcctccgtccagccctgaaacccgaggaggctccttcttccgtctgtccccgggtctcctaggggacggggctgtgaaagctggtgtggagggagaagcgagtgtggtccggagaaagaaggcgtggagaagagggagggagcgagagcgagagaataaatatataaataaatacgagaacgaaatccactccgcagtctccgggctcggaaactttggccccgagcgccagagcgccagagcgcgagagcgcggcgctcgccactctgaggctggcggcctcgattccggccgcgttcccccggcccccctccgccgcggggcctggtctccgggttctgccaggcgcatcagcccgcacaacttctggccgaggccagccggcagaggcggacttggggttggagtgt

SEQ ID NO: 36 - DNA, 1050 bp, synthetic construct
  source 1..1050: mol_type = other DNA
agcagctgggggcatttatgggccttcctataaacttctcctccagcagctggtcacaaagctggttaatctcccagagtgctcagcttaaaacccgtgactcacagcacagccagtgtgggggagggggtggctgcctccaatacgtggcgcccagagtcagctgttctggggccttctctggtttctccaactgagctagagactccgagagcagccgtgggcgacctgggggccggggactcggcggatccggcccattcatcaaaggaaggcactgggacaaagttggccccctccccttctcccccagcccccaactcgccgggatcggctccctctcttttcacggcggttcaaattctcccccaaaacgaggtcgagccggggattagaaaagagggaaaaaaatattcgttggcgatttcttttcgttattggtcctccgctcccttgcgccctggcccccttccctcctcatgaataaaagaaaagtccgaacctatcagatcgcgctcactgcctcctcctcctcctcctcctcgtacttcggtgaacacttttgcacaacttacccagctgatcactcgcgccccctcgcttttccatttctctttccccaaccccgtcaccccctgtctcctccgtccagccctgaaacccgaggaggctccttcttccgtctgtccccgggtctcctaggggacggggctgtgaaagctggtgtggagggagaagcgagtgtggtccggagaaagaaggcgtggagaagagggagggagcgagagcgagagaataaatatataaataaatacgagaacgaaatccactccgcagtctccgggctcggaaactttggccccgagcgccagagcgccagagcgcgagagcgcggcgctcgccactctgaggctggcggcctcgattctggccgcgttcccccggcccccctccgccgcggggcctggtctccgggttctgccaggcgcatcagcccgcacaacttctggccgaggccagccggcagaggcggacttggggttggagtgt

SEQ ID NO: 37 - DNA, 12 bp, synthetic construct
  source 1..12: mol_type = other DNA
atcccctcccca

SEQ ID NO: 38 - DNA, 19 bp, synthetic construct
  source 1..19: mol_type = other DNA
gccgagagagggcgggcag

SEQ ID NO: 39 - DNA, 12 bp, synthetic construct
  source 1..12: mol_type = other DNA
gtcggctgcact

SEQ ID NO: 40 - DNA, 14 bp, synthetic construct
  source 1..14: mol_type = other DNA
  misc_difference 1: note = a, c, t, g, unknown or other
  misc_difference 2: note = a, c, t, g, unknown or other
  misc_difference 3: note = a, c, t, g, unknown or other
  misc_difference 14: note = a, c, t, g, unknown or other
nnnrrcarytgyyn

SEQ ID NO: 41 - DNA, 11 bp, synthetic construct
  source 1..11: mol_type = other DNA
  misc_difference 10: note = a, c, t, g, unknown or other
rrcagstgsng

SEQ ID NO: 42 - DNA, 12 bp, synthetic construct
  source 1..12: mol_type = other DNA
agtgcagccgac

SEQ ID NO: 43 - DNA, 10 bp, synthetic construct
  source 1..10: mol_type = other DNA
ggcaaagggg

SEQ ID NO: 44 - DNA, 12 bp, synthetic construct
  source 1..12: mol_type = other DNA
  misc_difference 10: note = a, c, t, g, unknown or other
gcagctgtcnct

SEQ ID NO: 45 - DNA, 20 bp, synthetic construct
  source 1..20: mol_type = other DNA
gccctgcctttgttctcatg

SEQ ID NO: 46 - DNA, 10 bp, synthetic construct
  source 1..10: mol_type = other DNA
cccctttgcc

SEQ ID NO: 47 - DNA, 18 bp, synthetic construct
  source 1..18: mol_type = other DNA
  misc_difference 15: note = a, c, t, g, unknown or other
  misc_difference 17: note = a, c, t, g, unknown or other
wgarycagctgyggncnk

SEQ ID NO: 48 - DNA, 16 bp, synthetic construct
  source 1..16: mol_type = other DNA
  misc_difference 1: note = a, c, t, g, unknown or other
  misc_difference 2: note = a, c, t, g, unknown or other
  misc_difference 3: note = a, c, t, g, unknown or other
  misc_difference 4: note = a, c, t, g, unknown or other
  misc_difference 15: note = a, c, t, g, unknown or other
  misc_difference 16: note = a, c, t, g, unknown or other
nnnntaatyrattann

SEQ ID NO: 49 - DNA, 10 bp, synthetic construct
  source 1..10: mol_type = other DNA
gccaaaggaa

SEQ ID NO: 50 - DNA, 12 bp, synthetic construct
  source 1..12: mol_type = other DNA
  misc_difference 1: note = a, c, t, g, unknown or other
  misc_difference 12: note = a, c, t, g, unknown or other
nccagctgtccn

SEQ ID NO: 51 - DNA, 10 bp, synthetic construct
  source 1..10: mol_type = other DNA
ttcctttggc

SEQ ID NO: 52 - DNA, 12 bp, synthetic construct
  source 1..12: mol_type = other DNA
ggaggcgggggc

SEQ ID NO: 53 - DNA, 12 bp, synthetic construct
  source 1..12: mol_type = other DNA
gcccccgcctcc

SEQ ID NO: 54 - DNA, 12 bp, synthetic construct
  source 1..12: mol_type = other DNA
tggggaggggat

SEQ ID NO: 55-59 - skipped (5 entries)

SEQ ID NO: 60 - DNA, 10 bp, synthetic construct
  source 1..10: mol_type = other DNA
taatyratta

SEQ ID NO: 61 - DNA, 19 bp, synthetic construct
  source 1..19: mol_type = other DNA
cttttccatttctctttcc

SEQ ID NO: 62 - skipped

SEQ ID NO: 63 - DNA, 13 bp, synthetic construct
  source 1..13: mol_type = other DNA
tgcagctgtctct

SEQ ID NO: 64 - DNA, 601 bp, synthetic construct
  source 1..601: mol_type = other DNA
gcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaact

SEQ ID NO: 65 - DNA, 10 bp, synthetic construct
  source 1..10: mol_type = other DNA
tgcacctgct

SEQ ID NO: 66 - DNA, 10 bp, synthetic construct
  source 1..10: mol_type = other DNA
gcacctgctg

SEQ ID NO: 67 - DNA, 15 bp, synthetic construct
  source 1..15: mol_type = other DNA
cacacctgctccggc

SEQ ID NO: 68 - DNA, 15 bp, synthetic construct
  source 1..15: mol_type = other DNA
gccggagcaggtgtg

SEQ ID NO: 69 - DNA, 15 bp, synthetic construct
  source 1..15: mol_type = other DNA
gacagttgctgcatg

SEQ ID NO: 70 - DNA, 16 bp, synthetic construct
  source 1..16: mol_type = other DNA
catgcagcaactgtct

SEQ ID NO: 71 - DNA, 14 bp, synthetic construct
  source 1..14: mol_type = other DNA
ggagacagttgctg

SEQ ID NO: 72 - DNA, 15 bp, synthetic construct
  source 1..15: mol_type = other DNA
atgcagcaactgtct

SEQ ID NO: 73 - DNA, 14 bp, synthetic construct
  source 1..14: mol_type = other DNA
agacagttgctgca

SEQ ID NO: 74 - DNA, 14 bp, synthetic construct
  source 1..14: mol_type = other DNA
tgcagcaactgtct

SEQ ID NO: 75 - DNA, 20 bp, synthetic construct
  source 1..20: mol_type = other DNA
gccctgcctttgttctcatg

SEQ ID NO: 76 - DNA, 10 bp, synthetic construct
  source 1..10: mol_type = other DNA
agcagctggg

SEQ ID NO: 77 - DNA, 10 bp, synthetic construct
  source 1..10: mol_type = other DNA
cccagcygct

SEQ ID NO: 78 - DNA, 14 bp, synthetic construct
  source 1..14: mol_type = other DNA
cagcagctggtcac

SEQ ID NO: 79 - DNA, 14 bp, synthetic construct
  source 1..14: mol_type = other DNA
gtgaccagctgctg

SEQ ID NO: 80 - DNA, 11 bp, synthetic construct
  source 1..11: mol_type = other DNA
agcagctgggg

SEQ ID NO: 81 - DNA, 15 bp, synthetic construct
  source 1..15: mol_type = other DNA
agcagctggtcacaa

SEQ ID NO: 82 - DNA, 15 bp, synthetic construct
  source 1..15: mol_type = other DNA
ttgtgaccagctgct

SEQ ID NO: 83-84 - skipped (2 entries)

SEQ ID NO: 85 - DNA, 13 bp, synthetic construct
  source 1..13: mol_type = other DNA
tggctgcctccaa

SEQ ID NO: 86 - DNA, 10 bp, synthetic construct
  source 1..10: mol_type = other DNA
ggcagctgct

SEQ ID NO: 87 - DNA, 10 bp, synthetic construct
  source 1..10: mol_type = other DNA
tgcagctgct

SEQ ID NO: 88 - DNA, 10 bp, synthetic construct
  source 1..10: mol_type = other DNA
ggcagctgct

SEQ ID NO: 89 - DNA, 10 bp, synthetic construct
  source 1..10: mol_type = other DNA
agcagctgcc

SEQ ID NO: 90 - DNA, 10 bp, synthetic construct
  source 1..10: mol_type = other DNA
gcagctgctg

SEQ ID NO: 91 - DNA, 10 bp, synthetic construct
  source 1..10: mol_type = other DNA
gcagctgctt

SEQ ID NO: 92 - DNA, 10 bp, synthetic construct
  source 1..10: mol_type = other DNA
gggcagctgc

SEQ ID NO: 93 - DNA, 10 bp, synthetic construct
  source 1..10: mol_type = other DNA
gcagctgccc

SEQ ID NO: 94 - DNA, 15 bp, synthetic construct
  source 1..15: mol_type = other DNA
actggggcagctgct

SEQ ID NO: 95 - DNA, 15 bp, synthetic construct
  source 1..15: mol_type = other DNA
ggggctgcagctgct

SEQ ID NO: 96 - DNA, 15 bp, synthetic construct
  source 1..15: mol_type = other DNA
ggcagctgctggggg

SEQ ID NO: 97 - DNA, 15 bp, synthetic construct
  source 1..15: mol_type = other DNA
cccccagcagctgcc

SEQ ID NO: 98 - DNA, 15 bp, synthetic construct
  source 1..15: mol_type = other DNA
tgcagctgcttaatc

SEQ ID NO: 99 - DNA, 15 bp, synthetic construct
  source 1..15: mol_type = other DNA
gattaagcagctgca

SEQ ID NO: 100 - DNA, 14 bp, synthetic construct
  source 1..14: mol_type = other DNA
tggggcagctgctg

SEQ ID NO: 101 - DNA, 14 bp, synthetic construct
  source 1..14: mol_type = other DNA
ggctgcagctgctt

SEQ ID NO: 102 - DNA, 14 bp, synthetic construct
  source 1..14: mol_type = other DNA
gggcagctgctggg

SEQ ID NO: 103 - DNA, 14 bp, synthetic construct
  source 1..14: mol_type = other DNA
cccagcagctgccc

SEQ ID NO: 104 - DNA, 11 bp, synthetic construct
  source 1..11: mol_type = other DNA
ggcagctgctg

SEQ ID NO: 105 - DNA, 11 bp, synthetic construct
  source 1..11: mol_type = other DNA
ctgcagctgct

SEQ ID NO: 106 - DNA, 11 bp, synthetic construct
  source 1..11: mol_type = other DNA
agcagctgcag

SEQ ID NO: 107 - DNA, 10 bp, synthetic construct
  source 1..10: mol_type = other DNA
gggcagctgc

SEQ ID NO: 108 - DNA, 10 bp, synthetic construct
  source 1..10: mol_type = other DNA
ctgcagctgc

SEQ ID NO: 109 - DNA, 10 bp, synthetic construct
  source 1..10: mol_type = other DNA
gcagctgctg

SEQ ID NO: 110 - DNA, 10 bp, synthetic construct
  source 1..10: mol_type = other DNA
cagcagctgc

SEQ ID NO: 111 - DNA, 10 bp, synthetic construct
  source 1..10: mol_type = other DNA
gcagctgctt

SEQ ID NO: 112 - DNA, 36 bp, synthetic construct
  source 1..36: mol_type = other DNA
actggggcagctgctgggggctgcagctgcttaatc

SEQ ID NO: 113 - DNA, 30 bp, synthetic construct
  source 1..30: mol_type = other DNA
tggggcagctgctgggggctgcagctgctt

SEQ ID NO: 114 - DNA, 20 bp, synthetic construct
  source 1..20: mol_type = other DNA
ggggctgcagctgcttaatc

SEQ ID NO: 115 - DNA, 10 bp, synthetic construct
  source 1..10: mol_type = other DNA
ggcaggtgca

SEQ ID NO: 116 - DNA, 10 bp, synthetic construct
  source 1..10: mol_type = other DNA
tgcacctgcc

SEQ ID NO: 117 - DNA, 10 bp, synthetic construct
  source 1..10: mol_type = other DNA
gggcaggtgc

SEQ ID NO: 118 - DNA, 10 bp, synthetic construct
  source 1..10: mol_type = other DNA
gcacctgccc

SEQ ID NO: 119 - DNA, 11 bp, synthetic construct
  source 1..11: mol_type = other DNA
ggcaggtgcag

SEQ ID NO: 120 - DNA, 15 bp, synthetic construct
  source 1..15: mol_type = other DNA
ccatttatagcccca

SEQ ID NO: 121 - DNA, 15 bp, synthetic construct
  source 1..15: mol_type = other DNA
tggggctataaatgg

SEQ ID NO: 122 - DNA, 10 bp, synthetic construct
  source 1..10: mol_type = other DNA
cccagctgcc

SEQ ID NO: 123 - DNA, 10 bp, synthetic construct
  source 1..10: mol_type = other DNA
ccagctgccc

SEQ ID NO: 124 - DNA, 11 bp, synthetic construct
  source 1..11: mol_type = other DNA
ccccagctgcc

SEQ ID NO: 125 - DNA, 11 bp, synthetic construct
  source 1..11: mol_type = other DNA
ggcagctgggg

SEQ ID NO: 126 - DNA, 10 bp, synthetic construct
  source 1..10: mol_type = other DNA
gggcagctgt

SEQ ID NO: 127 - DNA, 14 bp, synthetic construct
  source 1..14: mol_type = other DNA
ctgggcagctgtca

SEQ ID NO: 128 - DNA, 14 bp, synthetic construct
  source 1..14: mol_type = other DNA
gggcagctgtcact

SEQ ID NO: 129 - DNA, 15 bp, synthetic construct
  source 1..15: mol_type = other DNA
agtggacagctgccc

SEQ ID NO: 130 - DNA, 15 bp, synthetic construct
  source 1..15: mol_type = other DNA
tcctgggcagctgtc

SEQ ID NO: 131 - DNA, 15 bp, synthetic construct
  source 1..15: mol_type = other DNA
ggcagctgtcactgg

SEQ ID NO: 132 - DNA, 15 bp, synthetic construct
  source 1..15: mol_type = other DNA
ccagtgacagctgcc

SEQ ID NO: 133 - DNA, 12 bp, synthetic construct
  source 1..12: mol_type = other DNA
gcagctgtcact

SEQ ID NO: 134 - DNA, 20 bp, synthetic construct
  source 1..20: mol_type = other DNA
tcctgggcagctgtcactgg

SEQ ID NO: 135 - DNA, 12 bp, synthetic construct
  source 1..12: mol_type = other DNA
gatgcagtggta

SEQ ID NO: 136 - DNA, 16 bp, synthetic construct
  source 1..16: mol_type = other DNA
acaataatcaattaca

SEQ ID NO: 137 - DNA, 16 bp, synthetic construct
  source 1..16: mol_type = other DNA
aataatcaattacaat

SEQ ID NO: 138 - DNA, 16 bp, synthetic construct
  source 1..16: mol_type = other DNA
attgtaattgattatt

SEQ ID NO: 139 - DNA, 10 bp, synthetic construct
  source 1..10: mol_type = other DNA
taatcaatta

SEQ ID NO: 140 - DNA, 10 bp, synthetic construct
  source 1..10: mol_type = other DNA
tgcctttgca

SEQ ID NO: 141 - DNA, 12 bp, synthetic construct
  source 1..12: mol_type = other DNA
accagctgtcca

SEQ ID NO: 142 - DNA, 18 bp, synthetic construct
  source 1..18: mol_type = other DNA
atgcccacaataatcaat

SEQ ID NO: 143 - DNA, 18 bp, synthetic construct
  source 1..18: mol_type = other DNA
attgattattgtgggcat

SEQ ID NO: 144 - DNA, 12 bp, synthetic construct
  source 1..12: mol_type = other DNA
gaactcagtctt

SEQ ID NO: 145 - DNA, 12 bp, synthetic construct
  source 1..12: mol_type = other DNA
aagactgagttc

SEQ ID NO: 146 - DNA, 11 bp, synthetic construct
  source 1..11: mol_type = other DNA
gctaattggag

SEQ ID NO: 147 - DNA, 11 bp, synthetic construct
  source 1..11: mol_type = other DNA
ctccaattagc

SEQ ID NO: 148 - DNA, 12 bp, synthetic construct
  source 1..12: mol_type = other DNA
ccaattaagggc

SEQ ID NO: 149 - DNA, 12 bp, synthetic construct
  source 1..12: mol_type = other DNA
gcccttaattgg

SEQ ID NO: 150 - DNA, 10 bp, synthetic construct
  source 1..10: mol_type = other DNA
ccaacctgac

SEQ ID NO: 151 - DNA, 12 bp, synthetic construct
  source 1..12: mol_type = other DNA
gaggttaattcc

SEQ ID NO: 152 - DNA, 12 bp, synthetic construct
  source 1..12: mol_type = other DNA
ccacccacctct

SEQ ID NO: 153 - DNA, 15 bp, synthetic construct
  source 1..15: mol_type = other DNA
ttgtcatgtaaagcg

SEQ ID NO: 154-155 - skipped (2 entries)

SEQ ID NO: 156 - DNA, 12 bp, synthetic construct
  source 1..12: mol_type = other DNA
ctgcaaggggca

SEQ ID NO: 157 - DNA, 12 bp, synthetic construct
  source 1..12: mol_type = other DNA
tgccccttgcag

SEQ ID NO: 158 - DNA, 10 bp, synthetic construct
  source 1..10: mol_type = other DNA
ggcaaaggtc

SEQ ID NO: 159 - DNA, 10 bp, synthetic construct
  source 1..10: mol_type = other DNA
gacctttgcc

SEQ ID NO: 160 - DNA, 10 bp, synthetic construct
  source 1..10: mol_type = other DNA
ggcgaagggg

SEQ ID NO: 161 - DNA, 10 bp, synthetic construct
  source 1..10: mol_type = other DNA
ccccttcgcc

SEQ ID NO: 162 - DNA, 18 bp, synthetic construct
  source 1..18: mol_type = other DNA
aaggtcacaggtgaggcg

SEQ ID NO: 163 - DNA, 18 bp, synthetic construct
  source 1..18: mol_type = other DNA
cgcctcacctgtgacctt

SEQ ID NO: 164 - DNA, 12 bp, synthetic construct
  source 1..12: mol_type = other DNA
aggggagggatg

SEQ ID NO: 165 - DNA, 12 bp, synthetic construct
  source 1..12: mol_type = other DNA
catccctcccct

SEQ ID NO: 166 - DNA, 12 bp, synthetic construct
  source 1..12: mol_type = other DNA
gagggcgggcag

SEQ ID NO: 167 - DNA, 12 bp, synthetic construct
  source 1..12: mol_type = other DNA
ctgcccgccctc

SEQ ID NO: 168 - DNA, 15 bp, synthetic construct
  source 1..15: mol_type = other DNA
ccctttttaacagcc

SEQ ID NO: 169 - DNA, 15 bp, synthetic construct
  source 1..15: mol_type = other DNA
ggctgttaaaaaggg

SEQ ID NO: 170 - DNA, 12 bp, synthetic construct
  source 1..12: mol_type = other DNA
ccccactgccga

SEQ ID NO: 171 - DNA, 12 bp, synthetic construct
  source 1..12: mol_type = other DNA
tcggcagtgggg

SEQ ID NO: 172 - DNA, 47 bp, synthetic construct
  source 1..47: mol_type = other DNA
catgctgcaaggggcaaaggtcacaggtgaggcgaaggggagggatg

SEQ ID NO: 173-174 - skipped (2 entries)

SEQ ID NO: 175 - DNA, 10 bp, synthetic construct
  source 1..10: mol_type = other DNA
gcgacctggg

SEQ ID NO: 176 - skipped

SEQ ID NO: 177 - DNA, 18 bp, synthetic construct
  source 1..18: mol_type = other DNA
agttggccccctcccctt

SEQ ID NO: 178 - DNA, 12 bp, synthetic construct
  source 1..12: mol_type = other DNA
ctcccccagccc

SEQ ID NO: 179 - DNA, 12 bp, synthetic construct
  source 1..12: mol_type = other DNA
gccccctcccct

SEQ ID NO: 180 - DNA, 12 bp, synthetic construct
  source 1..12: mol_type = other DNA
cccccagccctt

SEQ ID NO: 181 - DNA, 10 bp, synthetic construct
  source 1..10: mol_type = other DNA
cccagctgat

SEQ ID NO: 182 - DNA, 12 bp, synthetic construct
  source 1..12: mol_type = other DNA
gatcactcgcgc

SEQ ID NO: 183 - DNA, 12 bp, synthetic construct
  source 1..12: mol_type = other DNA
actcgcgccccc

SEQ ID NO: 184 - DNA, 12 bp, synthetic construct
  source 1..12: mol_type = other DNA
aaccccgtcacc

SEQ ID NO: 185 - DNA, 12 bp, synthetic construct
  source 1..12: mol_type = other DNA
cgccccctcgct

SEQ ID NO: 186 - skipped

SEQ ID NO: 187 - DNA, 11 bp, synthetic construct
  source 1..11: mol_type = other DNA
caccccctgtc

SEQ ID NO: 188 - DNA, 19 bp, synthetic construct
  source 1..19: mol_type = other DNA
cttttccatttctctttcc

SEQ ID NO: 189 - DNA, 11 bp, synthetic construct
  source 1..11: mol_type = other DNA
gcgccagagcg

SEQ ID NO: 190 - DNA, 14 bp, synthetic construct
  source 1..14: mol_type = other DNA
ctttggccccgagc

SEQ ID NO: 191 - DNA, 14 bp, synthetic construct
  source 1..14: mol_type = other DNA
tatataaataaata

SEQ ID NO: 192 - DNA, 15 bp, synthetic construct
  source 1..15: mol_type = other DNA
gtgtttgtttgtttg

SEQ ID NO: 193 - DNA, 13 bp, synthetic construct
  source 1..13: mol_type = other DNA
aaatatataaata

SEQ ID NO: 194 - DNA, 19 bp, synthetic construct
  source 1..19: mol_type = other DNA
cgccagagcgcgagagcgc

SEQ ID NO: 195 - DNA, 12 bp, synthetic construct
  source 1..12: mol_type = other DNA
gagcgcgggagt

SEQ ID NO: 196 - DNA, 12 bp, synthetic construct
  source 1..12: mol_type = other DNA
actcccgagctc

SEQ ID NO: 197 - DNA, 12 bp, synthetic construct
  source 1..12: mol_type = other DNA
caccccacctca

SEQ ID NO: 198 - DNA, 12 bp, synthetic construct
  source 1..12: mol_type = other DNA
aggggtgggggt

SEQ ID NO: 199 - DNA, 12 bp, synthetic construct
  source 1..12: mol_type = other DNA
acccccacccct

SEQ ID NO: 200 - DNA, 12 bp, synthetic construct
  source 1..12: mol_type = other DNA
gggggtgggggt

SEQ ID NO: 201 - DNA, 11 bp, synthetic construct
  source 1..11: mol_type = other DNA
acccccacccc

SEQ ID NO: 202 - DNA, 12 bp, synthetic construct
  source 1..12: mol_type = other DNA
tgtggaggggag

SEQ ID NO: 203 - DNA, 12 bp, synthetic construct
  source 1..12: mol_type = other DNA
ctcccctccaca

SEQ ID NO: 204 - DNA, 12 bp, synthetic construct
  source 1..12: mol_type = other DNA
aggggagggaga

SEQ ID NO: 205 - DNA, 12 bp, synthetic construct
  source 1..12: mol_type = other DNA
tctccctcccct

SEQ ID NO: 206 - DNA, 12 bp, synthetic construct
  source 1..12: mol_type = other DNA
gcggacgggaag

SEQ ID NO: 207 - DNA, 12 bp, synthetic construct
  source 1..12: mol_type = other DNA
cttcccgtccgc

SEQ ID NO: 208 - DNA, 12 bp, synthetic construct
  source 1..12: mol_type = other DNA
gtccccggcgtg

SEQ ID NO: 209 - DNA, 12 bp, synthetic construct
  source 1..12: mol_type = other DNA
cttcccggcacg

SEQ ID NO: 210-211 - skipped (2 entries)

SEQ ID NO: 212 - DNA, 12 bp, synthetic construct
  source 1..12: mol_type = other DNA
cccggcgtgcgc

SEQ ID NO: 213 - DNA, 12 bp, synthetic construct
  source 1..12: mol_type = other DNA
gcgcacgccggg

SEQ ID NO: 214 - DNA, 16 bp, synthetic construct
  source 1..16: mol_type = other DNA
ccagcttataatcgcg

SEQ ID NO: 215 - DNA, 10 bp, synthetic construct
  source 1..10: mol_type = other DNA
ctgcagccag

SEQ ID NO: 216-217 - skipped (2 entries)

SEQ ID NO: 218 - DNA, 12 bp, synthetic construct
  source 1..12: mol_type = other DNA
atcgcagcaggg

SEQ ID NO: 219 - DNA, 41 bp, synthetic construct
  source 1..41: mol_type = other DNA
gtccccggcgtgcgcaagaatggcggcccttcccggcacgg

SEQ ID NO: 220 - DNA, 13 bp, synthetic construct
  source 1..13: mol_type = other DNA
aaagagggaaaaa

SEQ ID NO: 221 - DNA, 12 bp, synthetic construct
  source 1..12: mol_type = other DNA
aaaaaaatattc

SEQ ID NO: 222 - DNA, 12 bp, synthetic construct
  source 1..12: mol_type = other DNA
attcgttggcga

SEQ ID NO: 223 - DNA, 28 bp, synthetic construct
  source 1..28: mol_type = other DNA
aaaaatattcgttggcgatttcttttcg

SEQ ID NO: 224 - DNA, 17 bp, synthetic construct
  source 1..17: mol_type = other DNA
ccctggcccccttccct

SEQ ID NO: 225 - DNA, 13 bp, synthetic construct
  source 1..13: mol_type = other DNA
gaataaaagaaaa

SEQ ID NO: 226 - skipped

SEQ ID NO: 227 - DNA, 12 bp, synthetic construct
  source 1..12: mol_type = other DNA
gctcccttgcgc

SEQ ID NO: 228 - DNA, 12 bp, synthetic construct
  source 1..12: mol_type = other DNA
cttccctcctca

SEQ ID NO: 229 - DNA, 15 bp, synthetic construct
  source 1..15: mol_type = other DNA
ctcctgtttaaacga

SEQ ID NO: 230 - skipped

SEQ ID NO: 231 - DNA, 12 bp, synthetic construct
  source 1..12: mol_type = other DNA
gtgcgcgccggc

SEQ ID NO: 232 - DNA, 12 bp, synthetic construct
  source 1..12: mol_type = other DNA
gcaggcgggaga

SEQ ID NO: 233 - DNA, 12 bp, synthetic construct
  source 1..12: mol_type = other DNA
tctcccgcctgc

SEQ ID NO: 234 - DNA, 12 bp, synthetic construct
  source 1..12: mol_type = other DNA
atcgcagcaggg

SEQ ID NO: 235 - DNA, 12 bp, synthetic construct
  source 1..12: mol_type = other DNA
tttgcagtggca

SEQ ID NO: 236 - DNA, 12 bp, synthetic construct
  source 1..12: mol_type = other DNA
atcaagtgcgcg

SEQ ID NO: 237 - DNA, 12 bp, synthetic construct
  source 1..12: mol_type = other DNA
cgcgcacttgat

SEQ ID NO: 238 - DNA, 12 bp, synthetic construct
  source 1..12: mol_type = other DNA
gatcaagtgcgc

SEQ ID NO: 239 - DNA, 12 bp, synthetic construct
  source 1..12: mol_type = other DNA
gcgcacttgatc

SEQ ID NO: 240 - DNA, 10 bp, synthetic construct
  source 1..10: mol_type = other DNA
tcaagtgcgc

SEQ ID NO: 241 - DNA, 10 bp, synthetic construct
  source 1..10: mol_type = other DNA
gcgcacttga

SEQ ID NO: 242 - DNA, 11 bp, synthetic construct
  source 1..11: mol_type = other DNA
cgcaggcggga

SEQ ID NO: 243 - DNA, 11 bp, synthetic construct
  source 1..11: mol_type = other DNA
tcccgcctgcg

SEQ ID NO: 244 - DNA, 36 bp, synthetic construct
  source 1..36: mol_type = other DNA
gatcaagtgcgcgccggcagccacgcaggcgggaga

SEQ ID NO: 245 - DNA, 12 bp, synthetic construct
  source 1..12: mol_type = other DNA
atcgcagtgggg

SEQ ID NO: 246 - DNA, 11 bp, synthetic construct
  source 1..11: mol_type = other DNA
ctctgtggcgt

SEQ ID NO: 247 - DNA, 11 bp, synthetic construct
  source 1..11: mol_type = other DNA
acgccacagag

SEQ ID NO: 248 - DNA, 12 bp, synthetic construct
  source 1..12: mol_type = other DNA
tgtggcgtgcaa

SEQ ID NO: 249 - DNA, 12 bp, synthetic construct
  source 1..12: mol_type = other DNA
ttgcacgccaca

SEQ ID NO: 250 - DNA, 12 bp, synthetic construct
  source 1..12: mol_type = other DNA
ggccaagttccc

SEQ ID NO: 251 - DNA, 12 bp, synthetic construct
  source 1..12: mol_type = other DNA
gggaacttggcc

SEQ ID NO: 252 - DNA, 11 bp, synthetic construct
  source 1..11: mol_type = other DNA
cccagatgccc

SEQ ID NO: 253 - DNA, 11 bp, synthetic construct
  source 1..11: mol_type = other DNA
gggcatctggg

SEQ ID NO: 254 - DNA, 11 bp, synthetic construct
  source 1..11: mol_type = other DNA
gctgcacttgg

SEQ ID NO: 255 - DNA, 12 bp, synthetic construct
  source 1..12: mol_type = other DNA
gctgcacttgga

SEQ ID NO: 256 - DNA, 12 bp, synthetic construct
  source 1..12: mol_type = other DNA
aagggagggggt

SEQ ID NO: 257 - DNA, 12 bp, synthetic construct
  source 1..12: mol_type = other DNA
accccctccctt

SEQ ID NO: 258 - DNA, 10 bp, synthetic construct
  source 1..10: mol_type = other DNA
ggaacctgat

SEQ ID NO: 259 - DNA, 14 bp, synthetic construct
  source 1..14: mol_type = other DNA
ggaccaataaaggc

SEQ ID NO: 260 - DNA, 15 bp, synthetic construct
  source 1..15: mol_type = other DNA
ttgtttataatttac

SEQ ID NO: 261 - DNA, 15 bp, synthetic construct
  source 1..15: mol_type = other DNA
gtaaattataaacaa

SEQ ID NO: 262 - DNA, 10 bp, synthetic construct
  source 1..10: mol_type = other DNA
tgcaaaggca

SEQ ID NO: 263 - DNA, 10 bp, synthetic construct
  source 1..10: mol_type = other DNA
tgcctttgca

SEQ ID NO: 264 - DNA, 12 bp, synthetic construct
  source 1..12: mol_type = other DNA
aaaggcagggct

SEQ ID NO: 265 - DNA, 12 bp, synthetic construct
  source 1..12: mol_type = other DNA
agccctgccttt

SEQ ID NO: 266 - DNA, 12 bp, synthetic construct
  source 1..12: mol_type = other DNA
cagggctggaga

SEQ ID NO: 267 - DNA, 12 bp, synthetic construct
  source 1..12: mol_type = other DNA
tctccagccctg

SEQ ID NO: 268 - DNA, 13 bp, synthetic construct
  source 1..13: mol_type = other DNA
tgcagctgtctct

SEQ ID NO: 269 - DNA, 10 bp, synthetic construct
  source 1..10: mol_type = other DNA
cacctttgca

SEQ ID NO: 270 - DNA, 12 bp, synthetic construct
  source 1..12: mol_type = other DNA
cctcccgcctgc

SEQ ID NO: 271 - DNA, 12 bp, synthetic construct
  source 1..12: mol_type = other DNA
ccgcctgcccct

SEQ ID NO: 272 - DNA, 12 bp, synthetic construct
  source 1..12: mol_type = other DNA
tgcccctcctcc

SEQ ID NO: 273 - DNA, 11 bp, synthetic construct
  source 1..11: mol_type = other DNA
ggccaattagg

SEQ ID NO: 274 - DNA, 11 bp, synthetic construct
  source 1..11: mol_type = other DNA
aggcccctgac

SEQ ID NO: 275 - DNA, 11 bp, synthetic construct
  source 1..11: mol_type = other DNA
tgacacctgga

SEQ ID NO: 276 - DNA, 12 bp, synthetic construct
  source 1..12: mol_type = other DNA
tgacacctggag

SEQ ID NO: 277 - DNA, 12 bp, synthetic construct
  source 1..12: mol_type = other DNA
tgacacctggag

SEQ ID NO: 278 - DNA, 10 bp, synthetic construct
  source 1..10: mol_type = other DNA
tgtcacttgc

SEQ ID NO: 279 - DNA, 12 bp, synthetic construct
  source 1..12: mol_type = other DNA
tgtcacttgctc

SEQ ID NO: 280 - DNA, 11 bp, synthetic construct
  source 1..11: mol_type = other DNA
ccgcagaagct

SEQ ID NO: 281 - DNA, 10 bp, synthetic construct
  source 1..10: mol_type = other DNA
agggccgcag

SEQ ID NO: 282 - DNA, 10 bp, synthetic construct
  source 1..10: mol_type = other DNA
ctgcggccct

SEQ ID NO: 283 - DNA, 56 bp, synthetic construct
  source 1..56: mol_type = other DNA
cctcccgcctgcccctcctcccctcagctcggccaattaggcccctgacacctgga

SEQ ID NO: 284 - DNA, 10 bp, synthetic construct
  source 1..10: mol_type = other DNA
tcccaggcac

SEQ ID NO: 285 - DNA, 10 bp, synthetic construct
  source 1..10: mol_type = other DNA
ccccaggcag

SEQ ID NO: 286 - DNA, 10 bp, synthetic construct
  source 1..10: mol_type = other DNA
atccaggcac

SEQ ID NO: 287 - DNA, 10 bp, synthetic construct
  source 1..10: mol_type = other DNA
atccaggcac

SEQ ID NO: 288 - DNA, 12 bp, synthetic construct
  source 1..12: mol_type = other DNA
gtcccagccaaa

SEQ ID NO: 289-292 - skipped (4 entries)

SEQ ID NO: 293 - DNA, 18 bp, synthetic construct
  source 1..18: mol_type = other DNA
agttggccccctcccctt

SEQ ID NO: 294 - DNA, 12 bp, synthetic construct
  source 1..12: mol_type = other DNA
ctcccccagccc

SEQ ID NO: 295 - DNA, 14 bp, synthetic construct
  source 1..14: mol_type = other DNA
ctttggccccgagc

SEQ ID NO: 296 - DNA, 12 bp, synthetic construct
  source 1..12: mol_type = other DNA
cgccccctcgct

SEQ ID NO: 297 - DNA, 12 bp, synthetic construct
  source 1..12: mol_type = other DNA
gaataaaagaaa

SEQ ID NO: 298 - DNA, 52 bp, synthetic construct
  source 1..52: mol_type = other DNA
gtggaggccaagttcccagatgcccagggtcggaggatcgcagtgggggaga

SEQ ID NO: 299 - DNA, 28 bp, synthetic construct
  source 1..28: mol_type = other DNA
aaaaatattcgttggcgatttcttttcg

SEQ ID NO: 300 - DNA, 10 bp, synthetic construct
  source 1..10: mol_type = other DNA
cccagctgat

SEQ ID NO: 301 - skipped

SEQ ID NO: 302 - DNA, 19 bp, synthetic construct
  source 1..19: mol_type = other DNA
cttttccatttctctttcc

SEQ ID NO: 303 - DNA, 19 bp, synthetic construct
  source 1..19: mol_type = other DNA
cgccagagcgcgagagcgc

SEQ ID NO: 304 - DNA, 17 bp, synthetic construct
  source 1..17: mol_type = other DNA
ccctggcccccttccct

SEQ ID NO: 305 - DNA, 15 bp, synthetic construct
  source 1..15: mol_type = other DNA
cctcctcctcctcct

SEQ ID NO: 306 - DNA, 450 bp, synthetic construct
  source 1..450: mol_type = other DNA
ctagcatgctgcccatgtaaggaggcaaggcctggggacacccgagatgcctggttataattaacccagacatgtggctgcccccccccccccaacacctgctgcctctaaaaataaccctgcatgccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagcgtcga

SEQ ID NO: 307 - DNA, 10 bp, synthetic construct
  source 1..10: mol_type = other DNA
aagcagctgc

(19) United States
(12) Patent Application Publication
(10) Pub. No.: US 2026/0306674 A1
(43) Pub. Date: Oct. 8, 2026

(54) NOVEL REGULATORY CASSETTES FOR SPECIFIC EXPRESSION OF GENES IN MUSCLE STEM CELLS

(71) Applicant: UNIVERSITY OF WASHINGTON, Seattle, WA (US)

(72) Inventor: Niclas BENGTSSON, Seattle, WA (US)

(73) Assignee: UNIVERSITY OF WASHINGTON, Seattle, WA (US)

(21) Appl. No.: 19/479,949

(22) PCT Filed: Apr. 30, 2024
(86) PCT No.: PCT/US2024/027061

§ 371 Date: Oct. 29, 2025

Related U.S. Application Data
(60) Provisional application No. 63/463,152, filed on May 1, 2023.

(51) Int. Cl. A61K 48/00; A61K 38/00; A61P 21/00; C07K 14/47; C12N 9/22; C12N 15/11; C12N 15/86

(52) U.S. Cl. CPC A61K 48/0058; A61P 21/00; C07K 14/4708; C12N 9/226; C12N 15/111; C12N 15/86; A61K 38/00; C12N 2310/20; C12N 2750/14143

(57) ABSTRACT

The technology disclosed herein relates to regulatory expression constructs (RC) to drive transgene expression in muscle stem cells (MuSCs). Herein, the MuSC-RC can be used for targeted transgene expression and/or gene editing specifically in MuSCs, which uniquely permits genomic modifications in MuSCs to be inherited by resulting progeny that either differentiate to repair injured muscle, or return to quiescence as MuSCs for future regenerative needs. The technology also relates to nucleic acid constructs comprising the MuSC-RC operatively linked to transgenes, and vectors, compositions and cells comprising the same. Aspects of the technology also relate to methods of expressing a transgene and/or gene editing in a muscle satellite cell using the MuSC-RC, as well as methods for treating a neuromuscular disease or disorder in a subject using the same.

### CROSS-REFERENCE TO RELATED APPLICATIONS

This application is a 35 U.S.C. § 371 National Phase Entry Application of International Application No. PCT/US2024/27061, filed on Apr. 30, 2024, which designates the U.S., and which claims benefit under 35 U.S.C. § 119(e) of U.S. Provisional Application No. 63/463,152 filed on May 1, 2023, the contents of each of which are incorporated herein by reference in their respective entireties.

### GOVERNMENT SUPPORT

This invention was made with government support under Grant No. HT9425-23-1-0575, awarded by the Department of Defense (DOD). The government has certain rights in the invention.

### FIELD OF THE INVENTION

Regulatory cassettes for specific expression of genes in muscle stem cells (muSC). The current disclosure relates to synthetic regulatory constructs comprising regulatory element (RE) nucleic acid sequences, in particular muscle-specific regulatory elements, that are capable of regulating the expression of a transgene specifically in muscle stem cells (muSC), including skeletal muscle satellite cells. The disclosure also relates to expression constructs, vectors and cells comprising such muscle-specific regulatory nucleic acid sequences, and to methods of their use. The regulatory nucleic acid sequences are of particular utility for gene therapy applications, including but not limited to gene editing, and also find utility in other areas such as treating a disease or disorder in which it useful for expression in the skeletal muscle as well as bioprocessing and biotechnology.

### SEQUENCE LISTING

The instant application contains a Sequence Listing that has been submitted in XML format via Patent Center and is hereby incorporated by reference in its entirety. Said XML copy, created on Apr. 25, 2024, is named “034186-000107WOPT_SL.xml” and is 273,197 bytes in size.

### BACKGROUND OF THE INVENTION

Gene therapies are currently showing great clinical promise for effective treatments of muscle degenerative diseases such as Duchenne muscular dystrophy (DMD), and novel gene editing (CRISPR) approaches show immense potential for restoring ‘normal’ dystrophin protein by correcting mutations in muscle cells in vivo. However, long-term therapeutic success will require safe and efficient treatment of muscle stem cells (satellite cells, or SCs); which would ensure continued (possibly lifelong) replenishment of muscle with cells capable of producing dystrophin in the event of muscle regeneration in response to trauma, exercise or normal muscle maintenance. Proof-of-principle studies using non-specific virally derived gene regulatory cassettes (RCs) have shown that SCs can be targeted, but there are currently no effective means to safely restrict editing in SCs to limit potential side-effects.

Gene editing has emerged as a uniquely promising approach for long-term correction of mutations that cause muscular dystrophies such as Duchenne muscular dystrophy (DMD).⁵ One advantage of gene editing is the ability to address mutations ‘directly’, thus preserving normal endogenous gene regulation of dystrophin isoforms.⁶ However, as muscle regeneration of the edited muscle occurs, the edited gene is diluted out, and thus the therapeutic effect can be short lived and/or temporary. Gene editing could be permanent if applied to muscle stem cells, termed satellite cells (SCs), as progeny of corrected SCs would contribute to the regeneration of damaged myofibers and thus continually replenish dystrophin expression to improve treated muscles. It has been previously reported that adeno-associated viral (AAV) vector delivery of CRISPR/Cas9^{2-4,6,7-10} can be used to correct dystrophin expression in animal models of DMD. However, the correction of dystrophin in muscle stem cells using a AAV vector with a CMV regulator cassette was limited, as despite targeting up to 60% of SCs,^{8,11-13} the therapeutic effect was largely unsuccessful, because less than 0.05% of SCs were corrected following delivery of AAV:CRISPR/Cas9.¹¹ Moreover, the therapeutic value of using AAV gene transfer to SCs has historically been viewed as minimal due to the rapid dilution of therapeutic vector genomes that would occur in dividing stem- and progenitor cells during regeneration of dystrophic skeletal muscle.

Therefore there remains a need for regulatory nucleic acids which are able to drive transgene expression specifically in muscle stem cells. In particular, there is a need for regulatory nucleic acid constructs and elements that drive transgene expression in muscle stem cells that can be incorporated into expression constructs and vectors for satellite-specific expression of one or more desired transgenes (e.g., a therapeutic transgene or a gene-editing system, e.g., using CRISPR/Cas9 or other gene-editing system) which can improve long term production of a therapeutic gene and/or improve the efficacy or duration of gene editing effects in muscles.

### SUMMARY OF THE INVENTION

The present disclosure addresses the lack of suitable technologies for selectively targeting transgene expression in muscle stem cells (MuSC), which are also referred to herein as satellite cells.

Without wishing to be bound by theory, the inventors had previously developed several methods to express therapeutic genes in striated muscle, with a primary focus on establishing effective treatments for Duchenne muscular dystrophy (DMD). These have focused on restoring expression of dystrophin in cardiac and skeletal muscles using Adeno-associated viral (AAV) vector-mediated gene transfer of synthetic dystrophins, or via editing of the endogenous dystrophin gene using CRISPR/Cas9. Moreover, in this prior work the inventors developed muscle-specific expression cassettes (MSECs) to ensure efficient and specific expression of AAV delivered genes in postmitotic striated muscles.

Herein, to improve on the muscle pathophysiology and/or to increase the sustained long-term therapeutic efficacy, the inventors have developed regulatory expression constructs (RC) to drive transgene expression in muscle stem cells (MuSCs). This approach is in contrast to existing treatment paradigms, as the therapeutic value of AAV gene transfer to MuSCs has historically been viewed as minimal due to the rapid dilution of therapeutic vector genomes that would occur in dividing stem- and progenitor cells during regeneration of dystrophic skeletal muscle. However, herein the inventors demonstrate the efficacy of using MuSC-RC to facilitate gene editing specifically in MuSCs, which uniquely permits genomic modifications in MuSCs to be inherited by resulting progeny that either differentiate to repair injured muscle, or return to quiescence as MuSCs for future regenerative needs.

Accordingly, the technology described herein relates to the development of a library of novel regulator constructs, (herein referred to as “RCs) with efficient and specific transcriptional activities in muscle stem cells (MuSC, also referred to herein as satellite cells (SC)), that can be used to express a transgene, including therapeutic genes or gene editing genes efficiently in MuSC. The technology disclosed herein is an improvement on the inventors' previous experience in using muscle-specific expression cassettes (MSECs) in combination with AAV-mediated gene editing to safely and efficiently correct dystrophin mutations in striated muscle.

Herein, to restrict transcriptional gene activity to MuSCs, the inventors discovered and identified key regulatory domains containing control elements responsible for expression of the bona fide SC marker Pax7 and the myogenic stem/progenitor cell marker MyoD. Select regulatory elements (referred to herein as “REs’) were assembled into AAV-size-compatible MuSC-RCs and sequence optimized to confer specific transgene expression in MuSCs and proliferating myogenic progenitors, exhibiting a wide range of activities both in vitro and in vivo. These MuSC-RCs have the capacity to be instrumental for long-term correction of muscle degenerative conditions that would benefit from direct treatment of muscle stem cells.

Accordingly, aspects of the technology disclosed herein relate to a library of novel MuSC-RCs with optimal transcription rates and sizes, which are compatible for transgene expression in viral vectors, including AAV, and are compatible with, e.g., CRISPR/Cas9 or other gene editing system packaging into AAV.

In some embodiments, a MuSC-RCs as described herein is useful for expressing therapeutic proteins in SCs and myogenic progenitors, including for the purpose of treating a subject with a disease or disorder. As an exemplary example only, a vector comprising a MuSC-RC can be used to drive transgene expression for the purpose of treating a subject with a disease or disorder, e.g., but not limited to, correcting one or more dystrophin mutations in SCs of dystrophic hosts. In some embodiments, it is envisioned that the resulting correction of SCs will translate into long-term amelioration of skeletal muscle pathophysiology in DMD, and that this approach could be valuable in combinatorial treatments to correct the dystrophin gene or function in differentiated skeletal- and cardiac muscle cells as well as in SCs. In one embodiment, gene editing in SCs using the MuSC-RCs to drive the gene editing machinery could be combined with gene replacement therapy involving, e.g., microdystrophin expression in mature muscle cells. In such embodiments, microdystrophin constructs can be driven, for example, by muscle-specific expression cassettes as known in the art.

In brief, the Muscle-Stem cell Specific Regulatory Cassettes (MuSC-RC) as disclosed herein are molecular switches composed of DNA that can be used to selectively produce virtually any protein or RNA in skeletal muscle stem cells, while making virtually no product in non-stem cell muscle cells (i.e., mature muscle cells). MuSC-RCs can be operatively linked to any transgene, e.g., cDNAs encoding any protein or RNA, and when these constructs are inserted (transduced) into muscle stem cells, the transgene-encoded protein or RNA product will be synthesized. MuSC-RC-mediated product levels can be varied over concentration ranges exceeding 1000-fold by modifying their individual DNA sequences. MuSC-RCs can thus be used to selectively manufacture virtually any protein or RNA in skeletal muscle stem cells (MuSC) over very broad concentration ranges. This MuSC-RC capacity can be applied to gene therapy treatments for neuromuscular diseases, cancer cachexia, and aging diseases, as well as for any other diseases for which factors produced and secreted by muscle cells would be beneficial. The latter could range from secreted polypeptide hormones & cytokines, extracellular matrix proteins, enzymes, clotting factors and antibodies to metabolites. MuSC-RC diven gene expression can also be used for gene expression of a transcript for immunization purposes, e.g., immunization against virtually any antigen, for a wide variety of veterinary and animal agricultural purposes, as well as for cell-based meat production.

Many clinical uses for MuSC-RCs will be for expression of transgenes for a neuromuscular disease gene therapies and/or CRISPR/Cas9 gene correction strategies in which their high muscle specificity & ability to modify muscle stem cells that generate skeletal muscle provides beneficial safety features.

Muscle-stem cell Specific Expression regulatory Cassettes (MuSC-RCs) containing the regulatory elements as disclosed herein can provide a >100-fold range of product levels in skeletal muscle stem cells, as compared to a lower level in non-stem cell muscle cells (i.e., differentiated muscle cells) or cardiac muscle cells. This can be beneficial for optimal gene therapy because natural levels of individual proteins & RNAs vary widely. Producing less than normal product levels leads to suboptimal therapy, whereas excess levels can be toxic. In some embodiments, MuSC-RE have also been miniaturized to facilitate efficient packaging of large cDNAs in AAV delivery vectors.

The technology disclosed herein relates to regulatory expression constructs (RC) to drive transgene expression in muscle stem cells (MuSCs). Herein, the MuSC-RC can be used for targeted transgene expression and/or gene editing specifically in MuSCs, which uniquely permits genomic modifications in MuSCs to be inherited by resulting progeny that either differentiate to repair injured muscle, or return to quiescence as MuSCs for future regenerative needs. The technology also relates to nucleic acid constructs comprising the MuSC-RC operatively linked to transgenes, and vectors, compositions and cells comprising the same. Aspects of the technology also relate to methods of expressing a transgene and/or gene editing in a muscle satellite cell using the MuSC-RC, as well as methods for treating a neuromuscular disease or disorder in a subject using the same.

Aspects of the technology disclosed herein relate to a muscle-stem cell specific regulatory nucleic acid cassette (MuSC-RC) for selectively regulating the expression of an operatively linked heterologous transgene in a muscle stem cell (mSC), the MuSC-RC comprising at least two regulatory elements (RE), wherein the two RE's are selected from: (i) at least two REs located in an untranslated region of the PAX7 gene, (ii) at least two REs located in an untranslated region in the MyoD1 gene, or (iii) at least one RE located in an untranslated region of the PAX7 gene, and at least one RE located in an untranslated region in the MyoD1 gene, wherein the two RE are located adjacent to each other and are recombinant with respect to each other. In some embodiments, the muscle stem cell (mSC) is a skeletal muscle satellite cell.

In some embodiments, the MuSC-RC as disclosed herein comprises, (i) a nucleic acid sequence comprising at least one regulatory element (RE) from the untranslated region of the human PAX7 gene, or a functional fragment thereof, and (ii) a nucleic acid sequence comprising at least one regulatory element (RE) from the untranslated region of the human of MYOD1 gene, or a functional fragment thereof. In such embodiments, such a MuSC-RC is a chimeric MuSC-RC, e.g., MD/Px. The RE from Pax7 or MyoD can be in any order. In one embodiment, a MuSC-RC can comprise (i) a nucleic acid sequence comprising at least two regulatory elements (RE) from the 3′ UTR of the human PAX7 gene, or a functional fragment thereof, or (ii) a nucleic acid sequence comprising at least two regulatory elements (RE) from the 5′ UTR of the human of MYOD1 gene, or a functional fragment thereof, or (iii) a nucleic acid sequence comprising at least one regulatory element (RE) from the 5′ UTR of the human PAX7 gene, or a functional fragment thereof, and at least one regulatory element (RE) from the 5′ UTR of the human MyoD1 gene.

In some embodiments, a MuSC-RC can comprise a RE from the human PAX7 gene selected from any of: SEQ ID NO: 1-16, or a nucleic acid sequence having at least 85% sequence identity thereto. In some embodiments, a MuSC-RC can comprise a RE from the human MyoD1 gene selected from any of: SEQ ID NO: 17-22, or a nucleic acid sequence having at least 85% sequence identity thereto.

In some embodiments, a MuSC-RC can comprise any one of: (i) a nucleic acid sequence comprising SEQ ID NO: 4 and SEQ ID NO: 5 (R4-Px7 and R5-Px7 from the human PAX7 gene), or a functional fragment thereof, or a sequence having at least 85% sequence identity to at least SEQ ID NO: 4 or SEQ ID NO:5 or a functional variant thereof, or (ii) a nucleic acid sequence comprising at least (i) SEQ ID NO: 18 (R2-MD) and SEQ ID NO: 21 (R5-MD) or (ii) SEQ ID NO: 21 (R5-MD) and at least one of SEQ ID NO: 19, SEQ ID NO: 20 (R3-MD, R4-MD) of the human of MYOD1 gene, or a functional fragment thereof, or a sequence having at least 55% sequence identity to at least SEQ ID NO: 18, SEQ ID NO: 21 or SEQ ID NO: 19, SEQ ID NO: 20, a functional variant thereof.

In some embodiments, when a MuSC-RC as disclosed herein is operatively linked to a target nucleic acid, it results in a higher expression of the target nucleic acid in skeletal muscle satellite cells as compared to the expression of a target nucleic acid operatively linked to a CK8e regulatory element having a sequence of SEQ ID NO: 306.

In some embodiments, a MuSC-RC can comprise a nucleic acid sequence comprising at least a R5 and R6 RE of the PAX7 gene, comprises SEQ ID NO: 15 (R15-Px7) or SEQ ID NO: 16 (R16-Px7), or a sequence having at least 85% sequence identity to at least SEQ ID NO: 15 or SEQ ID NO: 16.

In some embodiments, a MuSC-RC can comprise a nucleic acid sequence comprising at least a R5 and R6 RE of the PAX7 gene, further comprises any one or more of: (a) a nucleic acid sequence comprising at least SEQ ID NO: 1 (R1-Px7) or a sequence having at least 85% sequence identity to SEQ ID NO: 1, (b) a nucleic acid sequence comprising at least SEQ ID NO: 2 (R2-Px7) or a sequence having at least 85% sequence identity to SEQ ID NO: 2, (c) a nucleic acid sequence comprising at least SEQ ID NO: 3 (R3-Px7), or a sequence having at least 85% sequence identity to SEQ ID NO: 3, (d) a nucleic acid sequence comprising at least SEQ ID NO: 4 (R4-Px7), or a sequence having at least 85% sequence identity to SEQ ID NO: 4, (e) a nucleic acid sequence comprising at least SEQ ID NO: 7 (R7-Px7), or a sequence having at least 85% sequence identity to SEQ ID NO: 7, (f) a nucleic acid sequence comprising at least SEQ ID NO: 8 (R8-Px7), or a sequence having at least 85% sequence identity to SEQ ID NO: 8, (g) a nucleic acid sequence comprising at least SEQ ID NO: 9 (R9-Px7), or a sequence having at least 85% sequence identity to SEQ ID NO: 9, (h) a nucleic acid sequence comprising at least SEQ ID NO: 10 (R10-Px7), or a sequence having at least 85% sequence identity to SEQ ID NO: 10, (i) a nucleic acid sequence comprising at least SEQ ID NO: 11 (R 11-Px7), or a sequence having at least 85% sequence identity to SEQ ID NO: 11, (j) a nucleic acid sequence comprising at least SEQ ID NO: 12 (R12-Px7), or a sequence having at least 85% sequence identity to SEQ ID NO: 12, (k) a nucleic acid sequence comprising at least SEQ ID NO: 13 (R13-Px7), or a sequence having at least 85% sequence identity to SEQ ID NO: 13, or (1) a nucleic acid sequence comprising at least SEQ ID NO: 14 (R14-Px7), or a sequence having at least 85% sequence identity to SEQ ID NO: 7.

In some embodiments, a MuSC-RC as disclosed herein can comprise a nucleic acid sequence comprising at least a R5 and R6 RE of the PAX7 gene comprises, or consists essentially of, a nucleic acid sequence comprising at least SEQ ID NOs: 4-7 (R7, R6, R5, R4), or a sequence having at least 85% sequence identity to SEQ ID NOs: 4-7, and can optionally further comprises at least one RE selected from R4 or R7 or a sequence having at least 95% sequence identity thereto.

In some embodiments, a MuSC-RC as disclosed herein can comprises at least one RE selected from R1, R2 and R3 or a sequence having at least 95% sequence identity thereto. In some embodiments, a MuSC-RC as disclosed herein can comprise a nucleic acid sequence that comprises two REs selected from any one or more of Pax7 RE's selected from SEQ ID NO: 1-14 (R1-Px7 to R14-Px7), or a sequence having at least 85% sequence identity to SEQ ID NO: 1-14. In some embodiments, the MuSC-RC can further comprise one or more Pax7 REs selected from any one or more of: R1, R2, R3 or a portion thereof. In some embodiments, a MuSC-RC as disclosed herein can further comprise one or more Pax7 REs selected from any one or more of: R1, R2, R3, or a portion thereof.

In some embodiments, a MuSC-RC as disclosed herein does not comprise any of: R8-R14, or a portion thereof.

In some embodiments, a MuSC-RC as disclosed herein comprises a nucleic acid sequence that comprises a R2 or R5 RE from the human of MYOD1 gene, and can further comprises any one or more of: (a) a nucleic acid sequence comprising at least SEQ ID NO: 19 (R3-MD), or a sequence having at least 85% sequence identity to SEQ ID NO: 19, (b) a nucleic acid sequence comprising at least SEQ ID NO: 20 (R4-MD), or a sequence having at least 85% sequence identity to SEQ ID NO: 20.

In some embodiments, a MuSC-RC as disclosed herein can comprise a R2 or R5 RE from the human of MYOD1 gene, and further comprises a RE selected from any one or more of: (a) a nucleic acid sequence comprising at least R2-MD, R3-MD and R5-MD, or a portion thereof, (b) a nucleic acid sequence comprising at least R2-MD, R4-MD and R6-MD, or a portion thereof, (c) a nucleic acid sequence comprising at least R2-MD, R3-MD, R4-MD, and R5-MD, or a portion thereof, or (d) a nucleic acid sequence comprising at least R4 and R5, or a portion thereof. In some embodiments, a MuSC-RC as disclosed herein does not comprise R6 or a coding region of the human of MYOD1 gene.

In some embodiments, a MuSC-RC as disclosed herein can comprise at least one RE from 3′UTR of the human of MYOD1 gene, and at least one RE from the 5′ UTR of the human PAX7 gene, e.g., is directed to a chimeric Px/MD MuSC-RC as disclosed herein. In some embodiments, a chimeric MuSC-RC can comprise (i) R2 nucleic acid sequence of the human of MYOD1 gene (R2-MD), and (ii) R5-Px7 or R6-Px7, or both R5-Px7 and R6-Px7 nucleic acid sequence of the human PAX7 gene. In some embodiments, a chimeric MuSC-RC can comprise a nucleic acid sequence is selected from: (i) R2-MD of the human of MYOD1 gene and R15-Px7 of the human PAX7 gene (e.g., chimeric MDcPxM99), or (ii) R2-MD of the human of MYOD1 gene and R16-Px7 of the human PAX7 gene. (e.g., chimeric Px/MD-2 also referred to as MDcPxM455). In some embodiments, a MuSC-RC as disclosed herein has a maximal length of 1500 nucleotides.

Another aspect of the technology disclosed herein relates to a nucleic acid construct comprising the MuSC-RC as disclosed herein, operatively linked to a target nucleic acid sequence, where in some embodiments, a target nucleic acid sequence is encodes a nuclease, e.g., a CRISPR-associated nuclease. In some embodiments, a target nucleic acid sequence is selected from any of: a miRNA, antisense nucleic acid sequence, gene or nucleic acid sequence encoding a therapeutic polypeptide. In some embodiments, a target nucleic acid sequence is associated with a neuromuscular disease or disorder, and some embodiments, the expression of the target nucleic acid sequence reduces a pathological effect or symptom of a neuromuscular disease or disorder. In some embodiments, a target nucleic acid sequence encodes a therapeutic polypeptide or gene-editing construct useful for the treatment of a neuromuscular disease or disorder, e.g., any one of, but not limited to, a muscular dystrophy selected from at least one of the myotonic muscular dystrophies (DM1 or DM2), Duchenne muscular dystrophy, Becker muscular dystrophy, the limb-girdle muscular dystrophies, the facioscapulohumeral muscular dystrophies, the congenital muscular dystrophies, oculopharyngeal muscular dystrophy, distal muscular dystrophy, the desmin-related myopathies, fukyama muscular dystrophy, the FKRP-deficiencies, and Emery-Dreifuss muscular dystrophy. In some embodiments, a construct comprising a MuSC-RC operatively linked to a target nucleic acid sequence is present in a non-viral or viral vector, e.g., an AAV vector.

In some embodiments, a nucleic acid construct further comprises one or more guide RNA (gRNA) cassettes, as disclosed herein.

Another aspect of the technology as disclosed herein relates to a recombinant vector comprising a MuSC-RC as disclosed herein, or a nucleic acid construct comprising the MuSC-RC, operatively linked to a target nucleic acid sequence. In some embodiments, the vector is a selected from any of: AAV vector, non-viral DNA vector, close-circular DNA vectors.

Another aspect of the technology relates to a cell comprising a MuSC-RC as disclosed herein, or a recombinant vector comprising a MuSC-RC as disclosed herein.

Another aspect of the technology relates to a method of expressing a transgene, including a gene-editing transgene, in a skeletal muscle satellite cell, the method comprising transducing a muscle tissue with a vector comprising a MuSC-RC as disclosed herein.

Another aspect of the technology relates to a method of treating a subject with a neuromuscular disease or disorder, comprising administering to a subject with a neuromuscular disease or disorder a recombinant vector comprising a MuSC-RC as disclosed herein, or a cell comprising a MuSC-RC as disclosed herein.

Another aspect of the technology relates to a composition comprising the recombinant vector comprising a MuSC-RC as disclosed herein for the treatment of a subject with a neuromuscular disease or disorder.

Another aspect of the technology relates to the use of the recombinant vector comprising a MuSC-RC as disclosed herein for the preparation of a medicament for the treatment of a neuromuscular disease or disorder.

Safe and effective targeting of MuSCs using any one or more MuSC-RC as a disclosed herein can address current limitations of in vivo skeletal muscle gene editing, and can help realize the full potential of gene editing for many genetic muscle diseases.

The summary above is meant to illustrate, in a non-limiting manner, some of the embodiments, advantages, features, and uses of the technology disclosed herein. Other embodiments, advantages, features, and uses of the technology disclosed herein will be apparent from the Detailed Description, the Drawings, the Examples, and the Claims.

### BRIEF DESCRIPTION OF THE DRAWINGS

The present teachings described herein will be more fully understood from the following description of various illustrative embodiments, when read together with the accompanying drawings. It should be understood that the drawings described below are for illustration purposes only and are not intended to limit the scope of the present teachings in any way.

FIG. 1 shows results of the correction of dystrophin expression in mdx4cv mice following systemic AAV-mediated gene editing using a post-mitotic striated muscle specific gene regulatory cassette (CK8e). Shown are cross-sections from heart, tibialis anterior (TA), diaphragm (Dia), and soleus (Sol) muscles; stained for dystrophin at 4 weeks post-treatment.³

FIG. 2 is a schematic illustration showing the loss of dystrophin correction during skeletal muscle turnover. AAV transduction and dystrophin mutation correction in myonuclei restores sarcolemmal dystrophin expression. However, dystrophin expression is lost over time due to muscle turnover in the absence of mutation correction in SCs.

FIG. 3A-3D shows the temporal loss of dystrophin-correction in skeletal muscle of mdx4cv mice. FIG. 3A is a schematic of two AAV-vectors, (i) the Nuclease vector (N) expressing CK8-SaCas9, having a CK8 promoter, and (ii) the target vector (T) comprising dual-gRNAs (g-i51 and g-i53) and CMV-mCherry. SgRNAs target introns 51 & 53 (UP arrows) of the Dmd gene to excise exons 52 & 53 (containing a TAA stop mutation) and restore the ORF of dystrophin (Δ5253). FIG. 3B are images of heart & skeletal muscle (diaphragm (Dia), gastrocnemius (Gastroc), tibialis anterior TA) cross-sections depicting dystrophin+myofibers at 4-(left) & 18 weeks (right) post-systemic delivery into 2 week-old mdx mice. FIG. 3C and FIG. 3D shows semi-quantitative-(FIG. 3C), and digital PCR (FIG. 3D) analyses depict loss of vector genomes (Vgs) and Δ5253 (A) dystrophin correction due to turnover between 4- and 18 weeks post-treatment.14 Values too low to display are demarcated with #, ***P<0.001, ****P<0.0001.

FIG. 4A-4D shows that CRISPR gene editing (as outlined in FIG. 2) results in turnover-related reduction of editing in skeletal—(diaphragm & gastrocnemius muscles). FIG. 4A shows reduction of Δ5253 (Δ) dystrophin correction in edited skeletal muscle (diaphragm & gastrocnemius muscles), but not the heart between 4- & 18 weeks post-treatment, as quantified using digital PCR. FIG. 4B shows AAV-microdystrophin (μDys) co-delivery preserves skeletal muscle editing after 18 weeks. FIG. 4C shows robust production of corrected mRNA from relatively few myonuclei of cardiac muscle. FIG. 4D shows that stabilization of muscle turnover via μDys delivery preserves long-term expression of delivered genes as evidenced by continued expression of mCherry (d, top) in Soleus (So), tibialis anterior (TA), gastrocnemius (Ga), extensor digitorum longus (Edl), quadriceps (Qu), diaphragm (Dia) and heart (He); and dystrophin (Dys) in Dia and Gastric (d, lower), as detected using antibodies raised against the C-terminus of dystrophin (C-term). Note that cardiac expression of mCherry and Dys (not shown) is preserved regardless of μDys co-delivery as the heart does not regenerate.⁴

FIG. 5A-5B is a schematic illustration of how AAV-size-compatible RCs based on Pax7 (FIG. 5A) or MyoD1 (FIG. 5B) gene regulatory elements are used to edit quiescent SC vs. activated SCs, respectively.

FIG. 5A show that using a Pax7 regulatory cassette, edited (grey box) quiescent SCs give rise to pools of activated SCs and progenitors that differentiate into dystrophin-expressing myonuclei in regenerated myofibers, or return to quiescence (dashed arrow). FIG. 5B shows that MyoD regulatory cassette, AAV-targeting of transient pools of activated and proliferating SCs generates edited (grey box) SCs that differentiate into dystrophin-expressing myonuclei. Edited SCs can also return to quiescence (solid arrow) and contribute to future rounds of regeneration (dashed arrow). Note that AAV vectors and editing activity is rapidly diluted during cell division, hence reducing risks of unwanted editing or immunological effects associated with extended editing. Clear boxes indicate non-edited cells. Pax7 (and Myf5) expression is lost from the activated SC as the SCs commit further towards myogenic differentiation, followed by the loss of MyoD expression in concert with initiated expression of late differentiation markers (including Myogenin and MRF4). These myoblasts then fuse to repair or form multinucleated syncytial muscle fibers.

FIG. 6A-6B are schematic illustrations showing exemplary regulatory elements (RE) in PAX7 and MYOD1 genes, and their use in exemplary synthetic MuSC-RC. FIG. 6A is an illustration of the PAX7 (chromosome 1, Chr.1) and MYOD1 (chromosome 11, Chr.11) genetic loci with approximate locations of identified regulatory element (RE) regions of interest outlined. R1 in the PAX7 gene is referred to herein as “R1-Px7”, R2 in the PAX7 gene is referred to herein as “R2-Px7” and so forth. R1 in the MYOD1 gene is referred to herein as “R1-MD”, R2 in the MYOD1 gene is referred to herein as “R2-MD” and so forth. FIG. 6B shows for illustration purposes only, a selection of RE regions from PAX7 and/or MYOD1 assembled into exemplary MuSC-RC, showing various combinations and sizes for in vitro & in vivo analyses of specific transcriptional activity in quiescent vs. activated MuSCs. Exemplary MuSC-RC are shown comprising RE's from each of PAX7 or MYOD1 gene separately, and as chimeric combinations of Res selected from either PAX7 and MYOD1. The exemplary MuSC-RC's shown in FIG. 6B are for illustration purposes only, and other combinations of at least 2 RE's from any of the REs from PAX7 and/or MYOD1 are encompassed for use in a MuSC-RC as disclosed herein, e.g., with exemplary combinations disclosed in the Tables herein.

FIG. 7A-7D show in vitro quantification of MuSC-RC activity levels. FIG. 7A is a schematic illustration of dual-luciferase plasmid construct where the MuSC-RC being measured (referred to as SCRC in the construct) drives expression of firefly luciferase (F.Luc), and is separated from control CMV-Renilla luciferase (R.Luc) by an insulator segment and poly-adenylation (Poly.A) signal. FIG. 7B shows the results of the activity levels of MuSC-RCs (normalized to internal CMV-R.Luc) for MyoD1 (MD)- and Pax7 (Px)-based MuSC-RCs shown in FIG. 6B at 48 hours post Lipofectamine-mediated transfection into mouse L6 fibroblasts, day 4 proliferating myoblasts and day 7 differentiated myocytes, derived from primary SCs isolated from wildtype 4 week-old male mouse hindlimb muscles. This demonstrates that most SCRCs exhibit myogenic specificity, and that MyoD1-based SCRCs exhibit higher activity levels than Pax7-based RCs, possibly due to native Pax7 transcription being rapidly shut down following SC isolation. SCRCs MD-1 (MD-689), MD-2 (MD-619), MD-5 (MD-413), Px-6 (PxM455) and chimeric MD/Px-1 (MDcPxM455) are all active in both proliferating & differentiated muscle cells could be ideal. FIG. 7C shows the results of the activity levels of select MuSC-RCs (including the CK8e MSEC that exhibits high & specific transcriptional activity in postmitotic striated muscle in vivo)¹⁴ at 48 hours post-transfection of mouse primary myogenic stem- and progenitor cells at day: 0, 1, 2 and 4 following isolation. FIG. 7D shows the relative native transcript abundance in skeletal muscle of CKM, MyoD and Pax7.

FIG. 8 shows images of myogenic cultures of primary isolated MuSCs from Pax7GFP/Ail4(CAG-fl-STOP-fl-tdTomato) double-transgenic mice. Top row shows proliferating SCs expressing GFP from the Pax7 locus. Transient transfection with plasmids expressing Cre-recombinase (CRE) using CMV, MyoD1 (MD), or Pax7 (Px7) RCs results in tdTomato expression from the Rosa locus following CRE-mediated recombination (middle row). Bottom row shows subsequent differentiation of transfected progenitor cells yields numerous tdTomato+myotubes. Note, that fewer tdTomato+myotubes using Px7-CRE may reflect either a smaller ratio of cells with Px7-RC activity at time of transfection, or a narrower time window of active CRE expression as Pax7 activity is rapidly altered during in vitro culture of primary cells.

FIG. 9A-9B is a schematic of an exemplary AAV vector comprising a MuSC-RC as disclosed herein. FIG. 9A is a schematic illustration of CRE-expressing AAV vectors enabling high-throughput testing of SC-RCs. Downstream of the SC-RC, the 5′-end of CRE contains a nuclear localization signal (NLS) followed by an in-frame cloning spacer and a triple-nucleotide barcode that is unique to each AAV vector and SC-RC (excluding ATG, TAA, TGA and TAG). FIG. 9B shows the inserted intron separates the CRE cDNA and enables distinct quantification of mRNA vs. AAV vector genomes via NGS.

FIG. 10A-10B shows results of FACS sorting of Pax7^{GFP+} satellite cells. FIG. 10A shows FACS plots showing selection of cell populations based on GFP and tdTomato expression at 2 weeks post IM (2×10⁹ vg/vector/inj) or IV (6.7×10¹⁰ vg/vector/inj) delivery of pooled and barcoded AAV vectors expressing CRE. FACS analyses indicate 13.9% (IM) and 20% (IV) of Gfp+SCs express tdTomato. FIG. 10B shows representative semi-quantitative RT-PCR and PCR amplicons submitted for NGS. Note: bottom extra band represents unused primer duplexes from samples with low levels of mRNA expression. n=8 muscles (IM) and n=8 or 12 muscles (RO), pool muscles for SC-isolation and DNA/RNA extraction. Each treatment group provides a controlled ratio of SCRC-activity and vector copy number; but may not be comparable across groups due to amplicon sequencing of varying amounts of sorted cells and extracted DNA/RNA.

FIG. 11A-11B show muscle stem cell prevalence & targeting is reduced in mdx vs. WT muscle. FACS analysis of mice treated with the IM or IV AAV vectors used in FIG. 10A-10B. FIG. 11A shows the % of Pax+7 satellite cells is reduced, and % targeting satellite cells is reduced on mdx mice. FIG. 11B shows % of Pax7+/td Tomato+satellite cells (SCs) targeted after AAVMyo1:CRE delivery via IM or IV injection, showing that even in dystrophic muscle, the percent targeted SC does not reduce after 10 weeks delivered by IM or IV routes.

FIG. 12A-12B show in vivo SCRC activity in Pax7+SCs—CRE mRNA and CRE AAV vectors. FIG. 12A shows the SC activity of MyoD-based SCRCs in healthy and dystrophic muscle. FIG. 12B shows the SC activity of Pax7-based SCRCs in healthy and dystrophic muscle. Each treatment group is internally controlled (each RC receives part of 100%). Normalized activity against CMV-generated transcripts/vector genomes. Mdx 10 week data may not be reliable due to muscle turnover—loss of vector causing difficult nested amplification with ~70 cycles. Remember, expressing cre doesn't correct the disease—muscle injury and regeneration is ongoing and causes loss of AAV vector genomes and hence reduces measurable RC activity.

FIG. 13A-13B show results of systematic testing of MD659 vs. Px831 4 weeks post-tranduction, showing the specificity of selectively targeting muscle stem cells. FIG. 13A shows immunostaining (top row) or FACs sorting (lower row) showing the expression of tdTomato red in skeletal muscle tissues: gastrocnemius (Ga), diaphragm (Dia), tibialis anterior (TA) muscles, but not in cardiac muscle (heart, he) after IV injection of AAVMyol:CRE that comprises a MuSC-RC selected from: Px831, MD689 or MD619, as compared to the untreated control. Importantly, Px831 showed minimal to negligible expression in the heart (He). FIG. 13B shows % targeted SC, and shows that although mice transduced with AAV comprising the Px831 MuSC-RC had the lowest % of targeting SC, the expression was highly specific to skeletal muscle stem cells. That is—the inventors surprisingly discovered that Px831 gives rise to tdTomato expression in skeletal muscle, but not cardiac muscle; suggesting higher level of restriction to SCs that give rise to progeny that fuse to repair skeletal muscle. N=3 for each. Injected into dystrophic mdx males, analyzed for tdTomato+/Pax7GFP+SCs and overall muscle tdTomato expression at 4 weeks. MD619 appears to yield highest targeting of SCs following systemic AAV delivery and expression of Cre-recombinase, labeling over half of sorted SCs and resulting in significant expression of tdTomato in bulk striated muscle; either via SC-fusion to regenerating dystrophic muscle (remember Cre doesn't stabilize muscle injury) or via simultaneous expression in cardiomyocytes and myofibers.

FIG. 14 shows an exemplary use of MuSC-RC for gene editing using split-intein mediated expression of large base-editors in postmitotic muscle and muscle stem cells following dual-AAV delivery. Shown is a schematic of a dual-delivery system where the MuSC-RC regulates the expression of the N-terminal of spCas9 (D10A fragment), and the other AAV expresses the C-terminal half of the spCas9(D10A) is operatively linked to a CMV promoter. Only in myogenic stem cells and muscle progenitors will both of the split-inteins be expressed, leading to gene editing only in the muscle stem cell population. It is envisioned that the level of gene editing in satellite cells can be compared to the gene editing that occurs when a control promoter, e.g., non-stem cells muscle specific promoter is used, e.g., CK8e, to regulate the expression of the expression of the N-terminal of spCas9 (D10A fragment). Additionally, as an extra level of control, the CMV promoter operatively linked to the C-terminal half of the spCas9(D10A) can be replaced with a MuSC-RC as disclosed herein. In some embodiments, the MuSC-RC used can be the same MuSC-RC used to regulate the expression of the N-terminal of spCas9 (D10A fragment), thereby adding an extra level of specificity to target muscle stem cells.

### DETAILED DESCRIPTION OF THE INVENTION

The technology described herein is based on the discovery of a library of regulatory elements for use in muscle stem cell specific regulatory constructs (MuSC-RCs) with efficient and specific transcriptional activities in MuSCs that can be used to express therapeutic genes. This work builds on the inventors' prior experience in using muscle-specific expression cassettes (MSECs) in combination with AAV-mediated gene editing to safely and efficiently correct dystrophin mutations in striated muscle, as disclosed in US Application US20170362635 ('635 application), which is incorporated herein in its entirety by reference. However, in contrast to the '635 application, where muscle cells are targeted, herein the inventors have developed synthetic muscle stem-cell specific regulatory cassettes (MuSC-RC) to restrict transcriptional gene activity to muscle stem cells or muscle satellite cells (SCs). In particular, the inventors have identified key regulatory domains (also referred to as regulatory elements or “RE's”) containing control elements responsible for expression of the bona fide stem cell marker Pax7 and the myogenic stem/progenitor cell marker MyoD. Select regulatory elements were assembled into AAV-size-compatible MuSC-RCs and sequence optimized to confer specific transgene expression in SCs and proliferating myogenic progenitors, exhibiting a wide range of activities both in vitro and in vivo. These MuSC-RCs have the potential to be instrumental for long-term correction of muscle degenerative conditions that would benefit from direct treatment of muscle stem cells.

Therapeutic MuSC editing is particularly applicable to DMD, due to an abundance of proliferating muscle stem- and progenitor cells responding to continuous bouts of injury and regeneration within dystrophic skeletal muscles. Herein, use of muscle stem cell specific promoters and regulatory elements will lead to enhanced therapeutic efficacy and longevity following dystrophin gene editing in MuSCs or progenitors. Previous studies have reported that at least some MuSC populations or progeny thereof can be transduced by AAV vectors, however, these studies have relied on ubiquitous and/or non-specific gene regulatory cassettes (RCs) to control editing activity. However, non-specific editing approaches can be troublesome for use in patients, not merely based on immunogenicity, but particularly when targeting mitotically competent cells in which unintended editing side-effects could cause detrimental outcomes such as malignancies. Accordingly, the MuSC-specific RCs as disclosed herein provide focused gene expression in muscle stem cells which could minimize such outcomes by eliminating potential oncogenic editing of non-target genes or cell types.

To address the need for MuSC-active RCs, the inventors expanded a AAV-size-compatible library of muscle-specific RCs to include RCs with specific activities in muscle stem- and muscle progenitor cells, and limited activity in cardiac cells. Herein, the inventors demonstrate numerous synthetic MuSC-RCs derived from highly conserved MyoD1 and Pax7 gene regulatory sequences (two transcription factors involved in the activation or maintenance of quiescent MuSCs). Herein in the Examples, the inventors demonstrate several candidate MuSC-RCs with preferential activities in myogenic stem- and progenitor cells that are derived solely from MyoD1 or Pax7, as well as from chimeric combinations of regulatory elements from both genes.

### I. Muscle Stem Cell Regulatory Cassettes (MuSC-RC)

The technology disclosed herein relates to regulatory expression constructs (RC) to drive transgene expression in muscle stem cells (MuSCs). Such MuSC-RC's can be used to improve muscle pathophysiology and/or to increase the sustained long-term therapeutic efficacy by driving transgene expression specifically in muscle stem cells (MuSCs). Herein, the MuSC-RC cab be used for gene editing specifically in MuSCs, which uniquely permits genomic modifications in MuSCs to be inherited by resulting progeny that either differentiate to repair injured muscle, or return to quiescence as MuSCs for future regenerative needs.

In some embodiments, the technology disclosed herein relates to a muscle-stem cell specific regulatory nucleic acid cassette (MuSC-RC) for selectively regulating the expression of an operatively linked heterologous transgene in a muscle stem cell (mSC), the MuSC-RC comprising at least two regulatory elements (RE), wherein the two RE's are selected from: (i) at least two REs located in an untranslated region of the PAX7 gene, (ii) at least two REs located in an untranslated region in the MyoD1 gene, or (iii) at least one RE located in an untranslated region of the PAX7 gene, and at least one RE located in an untranslated region in the MyoD1 gene, wherein the two RE are located adjacent to each other and are recombinant with respect to each other. In some embodiments, the muscle stem cell (mSC) is a skeletal muscle satellite cell. In some embodiments, the MuSC-RC comprises (i) a nucleic acid sequence comprising at least one regulatory element (RE) from the untranslated region of the human PAX7 gene, or a functional fragment thereof, and (ii) a nucleic acid sequence comprising at least one regulatory element (RE) from the untranslated region of the human of MYOD1 gene, or a functional fragment thereof.

In some embodiments, the MuSC-RC comprises a nucleic acid sequence comprising at least two regulatory element (RE) from the untranslated region of the human PAX7 gene, or a functional fragment thereof, as disclosed herein. In some embodiments, the MuSC-RC comprises a nucleic acid sequence comprising at least two regulatory element (RE) from the untranslated region of the human MyoD gene, or a functional fragment thereof, as disclosed herein.

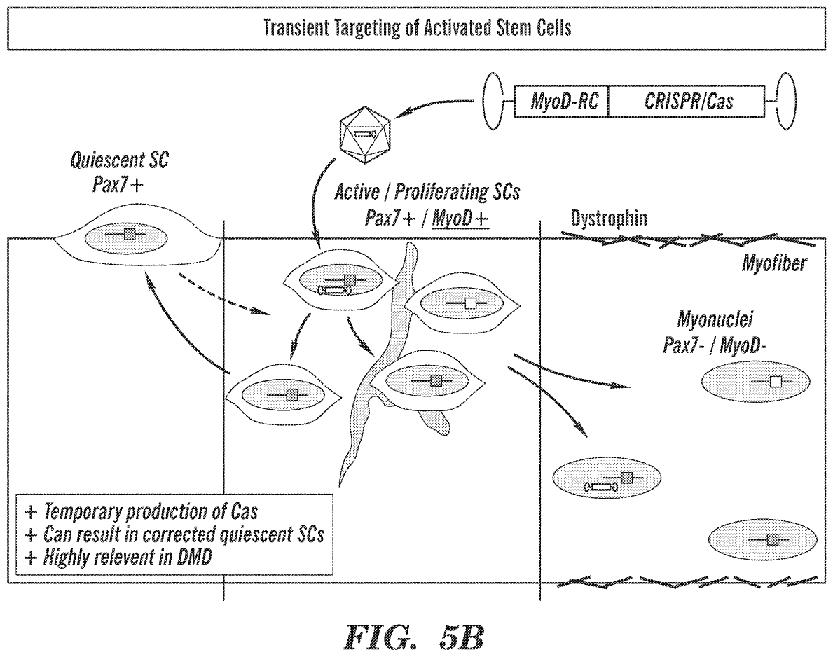

In some cases, the MuSC-RC promotes expression of an operatively linked transgene in a muscle stem cell. As used herein, muscle stem-cell specific means that the regulatory cassette has a preference for stem cells or satellite cells in the muscle. Such a regulatory construct can also regulate the expression of a transgene in other tissues, including other muscles, as long as there is an overall preference for expression in muscle stem cells, or muscle satellite cells. The MuSC-RCs disclosed herein can be active in skeletal muscle stem cells, including activated muscle stem cells and/or resting muscle stem cells. In some embodiments, the MuSC-RCs disclosed herein can drive expression of a transgene in early activated muscle stem cells. That is, referring to FIGS. 5A and 5B for illustration purposes, the MuSC-RC drive expression in muscle satellite cells that express Pax7 or MyoD, or both Pax7 and MyoD. In some embodiments, as illustrated in FIG. 5A, a MuSC-RC can drive expression in quiescent SC that are Pax7+. In some embodiments, the quiescent SC are Pax7+ and Pax3+. In some embodiments, the quiescent SC are Pax7+ and can also express both MyoD and Myf5 mRNA, however the mRNA is not transcribed to MyoD or Myf5 protein. In some embodiment, as illustrated in FIG. 5B, a MuSC-RC can drive expression in activated or proliferating satellite cells, which are activated in response to muscle injury, and where the activated SC express both Pax7 and MyoD (Pax7+/MyoD+). In some embodiments, activated SC are “early activated SC”, which co-express Pax7 with the myogenic regulatory factors Myf5 and MyoD (early activated). It is envisioned that the MuSC-RC can drive the expression of a transgene in a muscle stem cell (MuSC) as compared to a non-muscle cell, e.g., a fibroblast. Stated differently, a MuSC-RCs as disclosed herein has a preference for muscle cells, particularly muscle stem cells as compared to fibroblasts, or other non-muscle cells.

The term “muscle stem cell-specific” or “satellite cell-specific” refers to a MuSC-RC that has a preference for the expression of an operatively linked promoter in a muscle stem cell or satellite cells as opposed to a non-stem muscle cell. That is, while there may be some minimal expression in a muscle cell, it is envisioned that a MuSC-RC as disclosed herein has a higher expression in a muscle stem cell as compared to a non-stem muscle cell, and/or a non-muscle cell.

The term “muscle cell” or “myocyte” relates in the present to cells which are found in muscles (muscle tissue) or which are derived from muscle tissue. In some embodiments, muscle cells can be primary cells. In some embodiments, the muscle cells can in in vivo (e.g. in muscle tissue) or in vitro (e.g. in cell culture). Myocytes as found in muscle tissue are typically long, tubular cells that develop from myoblasts to form muscles in a process known as myogenesis. The term muscle cells or myocytes as used herein includes myocytes from skeletal muscle.

In some embodiments, a MuSC-RC comprises at least two regulator elements (REs), where the REs are located in tandem, or substantially adjacent to each other, and each RE is recombinant with respect to each other. This is also referred to herein as “heterologous with respect to each other”.

In any of the combinations of REs, or functional variants or fragments thereof, disclosed herein, some or all of the recited REs may suitably be positioned adjacent to one other in the MuSC-RE (i.e. without any intervening REs or other regulatory elements). The REs may be contiguous or non-contiguous (i.e. they can be positioned immediately adjacent to one another or they can be separated by a spacer or other sequence). The two or more RE's in a MuSC-RC are recombinant, in that, they are different from normally existing in nature, as they are not normally immediately adjacent to each other (i.e., intervening nucleic acid sequences between RE's have been removed). The RE's disclosed herein may be in any order.

In some embodiments, the REs, or functional variants thereof, are provided in the recited order and are adjacent to one another.

In some embodiments a MuSC-RC comprises two REs. where each RE is from a different gene, e.g., one RE is a Pax7 RE and one RE is a MYOD1 RE. In some embodiments, if a MuSC-RC comprises two REs from the same gene, each RE present in a MuSC-RC can be in close proximity to each other and has a portion of intervening endogenous nucleotides between the two RE's removed. Stated differently, where a MuSC-RC comprises two REs from the same gene, e.g., two REs from PAX7 gene, or two REs from MYOD1 gene, they are recombinant with respect to each other as a portion of the exogenous sequences located between the two RE's is removed. That is—the distance between two REs from the same gene (e.g., PAX7 or MYOD1 gene) is smaller than the distance between the two RE's in the respective endogenous PAX7 or MYOD gene.

In some embodiments, a RE for use in a MuSC-RE disclosed herein comprises one or more transcription factor binding site (TFBS) that promote expression in muscle stem cells. Exemplary consensus sequences of TFBS to specific transcription factors of interest are disclosed in Table 3. In some embodiments, each RE comprises at least 1, or 2, or 3, or 4, or 5, or 6, or 7 or more than 7 TFBS.

TABLE 3
Consensus sequences of target transcription factor binding sites (TFBS)

| | Consensus nucleic acid TFBS | | SEQ ID |
|---|---|---|---|
| Transcription factor | sequences | Size | NO: |
| MyoD1 binding | nscasctgyy | 10-mer | |
| MyoD binding | scasctgybn | 10-mer | |
| Myogenin | nnnnnarcagctgyn | 15-mer | |
| Myf6/mr4-directs diff. | nnnrrcarytgyyn | 14-mer | 40 |
| Myogenin binding | rrcagstgsng | 11-mer | 41 |
| (negative regulator) | | | |
| Myf5 binding - early | nnrcagctgy | 10-mer | |
| activation with MyoD | | | |
| TATA box new | nnnnnntataaannn | 15-mer | |
| MyoD1 binding site | GCAGCTGTCNCT | 12-mer | 44 |
| (jasper) | | | |
| pDES-RO1-R | GCCCTGCCTTTGTTCTCATG | 20-mer | 45 |
| CREB | nnygcagtngnn | 12-mer | |
| AP4 binding | wgarycagctgyggncnk | 18-mer | 47 |
| Pax7 binding | nnnntaatyrattann | 16-mer | 48 |
| TCF/Lef1 (Myf5 embryo | nncctttgnn | 10-mer | |
| paper) | | | |
| MCK promoter Ebox, | nccagctgtccn | 12-mer | 50 |
| binds MyoD1 | | | |
| AP-1/c-Fos | nnnactgagtnn | 12-mer | |
| Dix/Hox (Myf5 embryo, | nnccaattann | 11-mer | |
| with Fast-smad) | | | |
| Nkx2.5 | nnngttaattnn | 12-mer | |
| Mef3 | nnaacctgan | 10-mer | |
| Gli1 | ntgggtggn | 9-mer | |
| SP1 | nnncccgccnnn | 12-mer | |
| Ebox new | nnncacttgnnn | 12-mer | |
| Ebox 2 new | nnncacctgnn | 11-mer | |
| Myogenin (binds in | nnccaggcan | 10-mer | |
| Pax7) | | | |
| Pax7 only binding | taatyratta | | 60 |
| GC-Rich transcription | cttttccatttctctttcc | | 61 |
| binding (no TATA box) | | | |
| Runx | nnRccacagnn | | |
| mdMyoD1 binding | Tgcagctgtctct | | 63 |
| perfect | | | |

### II. Exemplary MuSC-RCs

As disclosed herein, a MuSC-RC as disclosed herein comprises at least two regulatory elements (REs). Each RE can be from the same gene, e.g., Pax7 or MyoD gene, or in alternative embodiments, at least one RE can be from the Pax7 gene and one from the MyoD1 gene.

### (a) Pax7-MuSC-RC (Px-MuSC-RC):

In some embodiments, a MuSC-RC is a Px-MuSC-RC and comprises at least two REs from the Pax7 gene (i.e., the MuSC does not comprise a RE from the MyoD1 gene). Exemplary combinations of 2 REs from Pax7 include, but are not limited to, the combinations shown in Table 1A.

TABLE 1A
Exemplary combinations of at least 2 RE selected from any of
the 16 PAX7 REs (120 options). These can be in any order,
and not necessarily in the order shown.
Table 1A: at least 2 RE selected from any of the R1-Px7 to R16-
Px16 RE; (which can be in any order, and not necessarily in the
order shown)

R1-Px7, R2-Px7
R1-Px7, R3-Px7
R1-Px7, R4-Px7
R1-Px7, R5-Px7
R1-Px7, R6-Px7
R1-Px7, R7-Px7
R1-Px7, R8-Px7
R1-Px7, R9-Px7
R1-Px7, R10-Px7
R1-Px7, R11-Px7
R1-Px7, R12-Px7
R1-Px7, R13-Px7
R1-Px7, R14-Px7
R1-Px7, R15-Px7
R1-Px7, R16-Px7
R2-Px7, R3-Px7
R2-Px7, R4-Px7
R2-Px7, R5-Px7
R2-Px7, R6-Px7
R2-Px7, R7-Px7
R2-Px7, R8-Px7
R2-Px7, R9-Px7
R2-Px7, R10-Px7
R2-Px7, R11-Px7
R2-Px7, R12-Px7
R2-Px7, R13-Px7
R2-Px7, R14-Px7
R2-Px7, R15-Px7
R2-Px7, R16-Px7
R3-Px7, R4-Px7
R3-Px7, R5-Px7
R3-Px7, R6-Px7
R3-Px7, R7-Px7
R3-Px7, R8-Px7
R3-Px7, R9-Px7
R3-Px7, R10-Px7
R3-Px7, R11-Px7
R3-Px7, R12-Px7
R3-Px7, R13-Px7
R3-Px7, R14-Px7
R3-Px7, R15-Px7
R3-Px7, R16-Px7
R4-Px7, R5-Px7
R4-Px7, R6-Px7
R4-Px7, R7-Px7
R4-Px7, R8-Px7
R4-Px7, R9-Px7
R4-Px7, R10-Px7
R4-Px7, R11-Px7
R4-Px7, R12-Px7
R4-Px7, R13-Px7
R4-Px7, R14-Px7
R4-Px7, R15-Px7
R4-Px7, R16-Px7
R5-Px7, R6-Px7
R5-Px7, R7-Px7
R5-Px7, R8-Px7
R5-Px7, R9-Px7
R5-Px7, R10-Px7
R5-Px7, R11-Px7
R5-Px7, R12-Px7
R5-Px7, R13-Px7
R5-Px7, R14-Px7
R5-Px7, R15-Px7
R5-Px7, R16-Px7
R6-Px7, R7-Px7
R6-Px7, R8-Px7
R6-Px7, R9-Px7
R6-Px7, R10-Px7
R6-Px7, R11-Px7
R6-Px7, R12-Px7
R6-Px7, R13-Px7
R6-Px7, R14-Px7
R6-Px7, R15-Px7
R6-Px7, R16-Px7
R7-Px7, R8-Px7
R7-Px7, R9-Px7
R7-Px7, R10-Px7
R7-Px7, R11-Px7
R7-Px7, R12-Px7
R7-Px7, R13-Px7
R7-Px7, R14-Px7
R7-Px7, R15-Px7
R7-Px7, R16-Px7
R8-Px7, R9-Px7
R8-Px7, R10-Px7
R8-Px7, R11-Px7
R8-Px7, R12-Px7
R8-Px7, R13-Px7
R8-Px7, R14-Px7
R8-Px7, R15-Px7
R8-Px7, R16-Px7
R9-Px7, R10-Px7
R9-Px7, R11-Px7
R9-Px7, R12-Px7
R9-Px7, R13-Px7
R9-Px7, R14-Px7
R9-Px7, R15-Px7
R9-Px7, R16-Px7
R10-Px7, R11-Px7
R10-Px7, R12-Px7
R10-Px7, R13-Px7
R10-Px7, R14-Px7
R10-Px7, R15-Px7
R10-Px7, R16-Px7
R11-Px7, R12-Px7
R11-Px7, R13-Px7
R11-Px7, R14-Px7
R11-Px7, R15-Px7
R11-Px7, R16-Px7
R12-Px7, R13-Px7
R12-Px7, R14-Px7
R12-Px7, R15-Px7
R12-Px7, R16-Px7
R13-Px7, R14-Px7
R13-Px7, R15-Px7
R13-Px7, R16-Px7
R14-Px7, R15-Px7
R14-Px7, R16-Px7
R15-Px7, R16-Px7

In some embodiments, a MuSC-RC is a Px-MuSC-RC and comprises at least three REs from the Pax7 gene (i.e., the MuSC does not comprise a RE from the MyoD 1 gene). Exemplary combinations of 3 REs from Pax7 include, but are not limited to, the combinations shown in Table 1B.

TABLE 1B
Exemplary combinations of a at least 3 RE selected from any of the R1-Px7
to P16-Px7. These can be in any order, and not necessarily in the order shown.
Table 1B: at least 3 RE selected from any of the R1-Px7 to R16-Px16 RE;
(which can be in any order, and not necessarily in the order shown)

| | | | |
|---|---|---|---|
| R1-Px7, R2-Px7, R3-Px7 | R2-Px7, R4-Px7, R11-Px7 | R3-Px7, R9-Px7, R12-Px7 | R5-Px7, R9-Px7, R15-Px7 |
| R1-Px7, R2-Px7, R4-Px7 | R2-Px7, R4-Px7, R12-Px7 | R3-Px7, R9-Px7, R13-Px7 | R5-Px7, R9-Px7, R16-Px7 |
| R1-Px7, R2-Px7, R5-Px7 | R2-Px7, R4-Px7, R13-Px7 | R3-Px7, R9-Px7, R14-Px7 | R5-Px7, R10-Px7, R11-Px7 |
| R1-Px7, R2-Px7, R6-Px7 | R2-Px7, R4-Px7, R14-Px7 | R3-Px7, R9-Px7, R15-Px7 | R5-Px7, R10-Px7, R12-Px7 |
| R1-Px7, R2-Px7, R7-Px7 | R2-Px7, R4-Px7, R15-Px7 | R3-Px7, R9-Px7, R16-Px7 | R5-Px7, R10-Px7, R13-Px7 |
| R1-Px7, R2-Px7, R8-Px7 | R2-Px7, R4-Px7, R16-Px7 | R3-Px7, R10-Px7, R11-Px7 | R5-Px7, R10-Px7, R14-Px7 |
| R1-Px7, R2-Px7, R9-Px7 | R2-Px7, R5-Px7, R6-Px7 | R3-Px7, R10-Px7, R12-Px7 | R5-Px7, R10-Px7, R15-Px7 |
| R1-Px7, R2-Px7, R10-Px7 | R2-Px7, R5-Px7, R7-Px7 | R3-Px7, R10-Px7, R13-Px7 | R5-Px7, R10-Px7, R16-Px7 |
| R1-Px7, R2-Px7, R11-Px7 | R2-Px7, R5-Px7, R8-Px7 | R3-Px7, R10-Px7, R14-Px7 | R5-Px7, R11-Px7, R12-Px7 |
| R1-Px7, R2-Px7, R12-Px7 | R2-Px7, R5-Px7, R9-Px7 | R3-Px7, R10-Px7, R15-Px7 | R5-Px7, R11-Px7, R13-Px7 |
| R1-Px7, R2-Px7, R13-Px7 | R2-Px7, R5-Px7, R10-Px7 | R3-Px7, R10-Px7, R16-Px7 | R5-Px7, R11-Px7, R14-Px7 |
| R1-Px7, R2-Px7, R14-Px7 | R2-Px7, R5-Px7, R11-Px7 | R3-Px7, R11-Px7, R12-Px7 | R5-Px7, R11-Px7, R15-Px7 |
| R1-Px7, R2-Px7, R15-Px7 | R2-Px7, R5-Px7, R12-Px7 | R3-Px7, R11-Px7, R13-Px7 | R5-Px7, R11-Px7, R16-Px7 |
| R1-Px7, R2-Px7, R16-Px7 | R2-Px7, R5-Px7, R13-Px7 | R3-Px7, R11-Px7, R14-Px7 | R5-Px7, R12-Px7, R13-Px7 |
| R1-Px7, R3-Px7, R4-Px7 | R2-Px7, R5-Px7, R14-Px7 | R3-Px7, R11-Px7, R15-Px7 | R5-Px7, R12-Px7, R14-Px7 |
| R1-Px7, R3-Px7, R5-Px7 | R2-Px7, R5-Px7, R15-Px7 | R3-Px7, R11-Px7, R16-Px7 | R5-Px7, R12-Px7, R15-Px7 |
| R1-Px7, R3-Px7, R6-Px7 | R2-Px7, R5-Px7, R16-Px7 | R3-Px7, R12-Px7, R13-Px7 | R5-Px7, R12-Px7, R16-Px7 |
| R1-Px7, R3-Px7, R7-Px7 | R2-Px7, R6-Px7, R7-Px7 | R3-Px7, R12-Px7, R14-Px7 | R5-Px7, R13-Px7, R14-Px7 |
| R1-Px7, R3-Px7, R8-Px7 | R2-Px7, R6-Px7, R8-Px7 | R3-Px7, R12-Px7, R15-Px7 | R5-Px7, R13-Px7, R15-Px7 |
| R1-Px7, R3-Px7, R9-Px7 | R2-Px7, R6-Px7, R9-Px7 | R3-Px7, R12-Px7, R16-Px7 | R5-Px7, R13-Px7, R16-Px7 |
| R1-Px7, R3-Px7, R10-Px7 | R2-Px7, R6-Px7, R10-Px7 | R3-Px7, R13-Px7, R14-Px7 | R5-Px7, R14-Px7, R15-Px7 |
| R1-Px7, R3-Px7, R11-Px7 | R2-Px7, R6-Px7, R11-Px7 | R3-Px7, R13-Px7, R15-Px7 | R5-Px7, R14-Px7, R16-Px7 |
| R1-Px7, R3-Px7, R12-Px7 | R2-Px7, R6-Px7, R12-Px7 | R3-Px7, R13-Px7, R16-Px7 | R5-Px7, R15-Px7, R16-Px7 |
| R1-Px7, R3-Px7, R13-Px7 | R2-Px7, R6-Px7, R13-Px7 | R3-Px7, R14-Px7, R15-Px7 | R6-Px7, R7-Px7, R8-Px7 |
| R1-Px7, R3-Px7, R14-Px7 | R2-Px7, R6-Px7, R14-Px7 | R3-Px7, R14-Px7, R16-Px7 | R6-Px7, R7-Px7, R9-Px7 |
| R1-Px7, R3-Px7, R15-Px7 | R2-Px7, R6-Px7, R15-Px7 | R3-Px7, R15-Px7, R16-Px7 | R6-Px7, R7-Px7, R10-Px7 |
| R1-Px7, R3-Px7, R16-Px7 | R2-Px7, R6-Px7, R16-Px7 | R4-Px7, R5-Px7, R6-Px7 | R6-Px7, R7-Px7, R11-Px7 |
| R1-Px7, R4-Px7, R5-Px7 | R2-Px7, R7-Px7, R8-Px7 | R4-Px7, R5-Px7, R7-Px7 | R6-Px7, R7-Px7, R12-Px7 |
| R1-Px7, R4-Px7, R6-Px7 | R2-Px7, R7-Px7, R9-Px7 | R4-Px7, R5-Px7, R8-Px7 | R6-Px7, R7-Px7, R13-Px7 |
| R1-Px7, R4-Px7, R7-Px7 | R2-Px7, R7-Px7, R10-Px7 | R4-Px7, R5-Px7, R9-Px7 | R6-Px7, R7-Px7, R14-Px7 |
| R1-Px7, R4-Px7, R8-Px7 | R2-Px7, R7-Px7, R11-Px7 | R4-Px7, R5-Px7, R10-Px7 | R6-Px7, R7-Px7, R15-Px7 |
| R1-Px7, R4-Px7, R9-Px7 | R2-Px7, R7-Px7, R12-Px7 | R4-Px7, R5-Px7, R11-Px7 | R6-Px7, R7-Px7, R16-Px7 |
| R1-Px7, R4-Px7, R10-Px7 | R2-Px7, R7-Px7, R13-Px7 | R4-Px7, R5-Px7, R12-Px7 | R6-Px7, R8-Px7, R9-Px7 |
| R1-Px7, R4-Px7, R11-Px7 | R2-Px7, R7-Px7, R14-Px7 | R4-Px7, R5-Px7, R13-Px7 | R6-Px7, R8-Px7, R10-Px7 |
| R1-Px7, R4-Px7, R12-Px7 | R2-Px7, R7-Px7, R15-Px7 | R4-Px7, R5-Px7, R14-Px7 | R6-Px7, R8-Px7, R11-Px7 |
| R1-Px7, R4-Px7, R13-Px7 | R2-Px7, R7-Px7, R16-Px7 | R4-Px7, R5-Px7, R15-Px7 | R6-Px7, R8-Px7, R12-Px7 |
| R1-Px7, R4-Px7, R14-Px7 | R2-Px7, R8-Px7, R9-Px7 | R4-Px7, R5-Px7, R16-Px7 | R6-Px7, R8-Px7, R13-Px7 |
| R1-Px7, R4-Px7, R15-Px7 | R2-Px7, R8-Px7, R10-Px7 | R4-Px7, R6-Px7, R7-Px7 | R6-Px7, R8-Px7, R14-Px7 |
| R1-Px7, R4-Px7, R16-Px7 | R2-Px7, R8-Px7, R11-Px7 | R4-Px7, R6-Px7, R8-Px7 | R6-Px7, R8-Px7, R15-Px7 |
| R1-Px7, R5-Px7, R6-Px7 | R2-Px7, R8-Px7, R12-Px7 | R4-Px7, R6-Px7, R9-Px7 | R6-Px7, R8-Px7, R16-Px7 |
| R1-Px7, R5-Px7, R7-Px7 | R2-Px7, R8-Px7, R13-Px7 | R4-Px7, R6-Px7, R10-Px7 | R6-Px7, R9-Px7, R10-Px7 |
| R1-Px7, R5-Px7, R8-Px7 | R2-Px7, R8-Px7, R14-Px7 | R4-Px7, R6-Px7, R11-Px7 | R6-Px7, R9-Px7, R11-Px7 |
| R1-Px7, R5-Px7, R9-Px7 | R2-Px7, R8-Px7, R15-Px7 | R4-Px7, R6-Px7, R12-Px7 | R6-Px7, R9-Px7, R12-Px7 |
| R1-Px7, R5-Px7, R10-Px7 | R2-Px7, R8-Px7, R16-Px7 | R4-Px7, R6-Px7, R13-Px7 | R6-Px7, R9-Px7, R13-Px7 |
| R1-Px7, R5-Px7, R11-Px7 | R2-Px7, R9-Px7, R10-Px7 | R4-Px7, R6-Px7, R14-Px7 | R6-Px7, R9-Px7, R14-Px7 |
| R1-Px7, R5-Px7, R12-Px7 | R2-Px7, R9-Px7, R11-Px7 | R4-Px7, R6-Px7, R15-Px7 | R6-Px7, R9-Px7, R15-Px7 |
| R1-Px7, R5-Px7, R13-Px7 | R2-Px7, R9-Px7, R12-Px7 | R4-Px7, R6-Px7, R16-Px7 | R6-Px7, R9-Px7, R16-Px7 |
| R1-Px7, R5-Px7, R14-Px7 | R2-Px7, R9-Px7, R13-Px7 | R4-Px7, R7-Px7, R8-Px7 | R6-Px7, R10-Px7, R11-Px7 |
| R1-Px7, R5-Px7, R15-Px7 | R2-Px7, R9-Px7, R14-Px7 | R4-Px7, R7-Px7, R9-Px7 | R6-Px7, R10-Px7, R12-Px7 |
| R1-Px7, R5-Px7, R16-Px7 | R2-Px7, R9-Px7, R15-Px7 | R4-Px7, R7-Px7, R10-Px7 | R6-Px7, R10-Px7, R13-Px7 |
| R1-Px7, R6-Px7, R7-Px7 | R2-Px7, R9-Px7, R16-Px7 | R4-Px7, R7-Px7, R11-Px7 | R6-Px7, R10-Px7, R14-Px7 |
| R1-Px7, R6-Px7, R8-Px7 | R2-Px7, R10-Px7, R11-Px7 | R4-Px7, R7-Px7, R12-Px7 | R6-Px7, R10-Px7, R15-Px7 |
| R1-Px7, R6-Px7, R9-Px7 | R2-Px7, R10-Px7, R12-Px7 | R4-Px7, R7-Px7, R13-Px7 | R6-Px7, R10-Px7, R16-Px7 |
| R1-Px7, R6-Px7, R10-Px7 | R2-Px7, R10-Px7, R13-Px7 | R4-Px7, R7-Px7, R14-Px7 | R6-Px7, R11-Px7, R12-Px7 |
| R1-Px7, R6-Px7, R11-Px7 | R2-Px7, R10-Px7, R14-Px7 | R4-Px7, R7-Px7, R15-Px7 | R6-Px7, R11-Px7, R13-Px7 |
| R1-Px7, R6-Px7, R12-Px7 | R2-Px7, R10-Px7, R15-Px7 | R4-Px7, R7-Px7, R16-Px7 | R6-Px7, R11-Px7, R14-Px7 |
| R1-Px7, R6-Px7, R13-Px7 | R2-Px7, R10-Px7, R16-Px7 | R4-Px7, R8-Px7, R9-Px7 | R6-Px7, R11-Px7, R15-Px7 |
| R1-Px7, R6-Px7, R14-Px7 | R2-Px7, R11-Px7, R12-Px7 | R4-Px7, R8-Px7, R10-Px7 | R6-Px7, R11-Px7, R16-Px7 |
| R1-Px7, R6-Px7, R15-Px7 | R2-Px7, R11-Px7, R13-Px7 | R4-Px7, R8-Px7, R11-Px7 | R6-Px7, R12-Px7, R13-Px7 |
| R1-Px7, R6-Px7, R16-Px7 | R2-Px7, R11-Px7, R14-Px7 | R4-Px7, R8-Px7, R12-Px7 | R6-Px7, R12-Px7, R14-Px7 |
| R1-Px7, R7-Px7, R8-Px7 | R2-Px7, R11-Px7, R15-Px7 | R4-Px7, R8-Px7, R13-Px7 | R6-Px7, R12-Px7, R15-Px7 |
| R1-Px7, R7-Px7, R9-Px7 | R2-Px7, R11-Px7, R16-Px7 | R4-Px7, R8-Px7, R14-Px7 | R6-Px7, R12-Px7, R16-Px7 |
| R1-Px7, R7-Px7, R10-Px7 | R2-Px7, R12-Px7, R13-Px7 | R4-Px7, R8-Px7, R15-Px7 | R6-Px7, R13-Px7, R14-Px7 |
| R1-Px7, R7-Px7, R11-Px7 | R2-Px7, R12-Px7, R14-Px7 | R4-Px7, R8-Px7, R16-Px7 | R6-Px7, R13-Px7, R15-Px7 |
| R1-Px7, R7-Px7, R12-Px7 | R2-Px7, R12-Px7, R15-Px7 | R4-Px7, R9-Px7, R10-Px7 | R6-Px7, R13-Px7, R16-Px7 |
| R1-Px7, R7-Px7, R13-Px7 | R2-Px7, R12-Px7, R16-Px7 | R4-Px7, R9-Px7, R11-Px7 | R6-Px7, R14-Px7, R15-Px7 |
| R1-Px7, R7-Px7, R14-Px7 | R2-Px7, R13-Px7, R14-Px7 | R4-Px7, R9-Px7, R12-Px7 | R6-Px7, R14-Px7, R16-Px7 |
| R1-Px7, R7-Px7, R15-Px7 | R2-Px7, R13-Px7, R15-Px7 | R4-Px7, R9-Px7, R13-Px7 | R6-Px7, R15-Px7, R16-Px7 |
| R1-Px7, R7-Px7, R16-Px7 | R2-Px7, R13-Px7, R16-Px7 | R4-Px7, R9-Px7, R14-Px7 | R7-Px7, R8-Px7, R9-Px7 |
| R1-Px7, R8-Px7, R9-Px7 | R2-Px7, R14-Px7, R15-Px7 | R4-Px7, R9-Px7, R15-Px7 | R7-Px7, R8-Px7, R10-Px7 |
| R1-Px7, R8-Px7, R10-Px7 | R2-Px7, R14-Px7, R16-Px7 | R4-Px7, R9-Px7, R16-Px7 | R7-Px7, R8-Px7, R11-Px7 |
| R1-Px7, R8-Px7, R11-Px7 | R2-Px7, R15-Px7, R16-Px7 | R4-Px7, R10-Px7, R11-Px7 | R7-Px7, R8-Px7, R12-Px7 |
| R1-Px7, R8-Px7, R12-Px7 | R3-Px7, R4-Px7, R5-Px7 | R4-Px7, R10-Px7, R12-Px7 | R7-Px7, R8-Px7, R13-Px7 |
| R1-Px7, R8-Px7, R13-Px7 | R3-Px7, R4-Px7, R6-Px7 | R4-Px7, R10-Px7, R13-Px7 | R7-Px7, R8-Px7, R14-Px7 |
| R1-Px7, R8-Px7, R14-Px7 | R3-Px7, R4-Px7, R7-Px7 | R4-Px7, R10-Px7, R14-Px7 | R7-Px7, R8-Px7, R15-Px7 |
| R1-Px7, R8-Px7, R15-Px7 | R3-Px7, R4-Px7, R8-Px7 | R4-Px7, R10-Px7, R15-Px7 | R7-Px7, R8-Px7, R16-Px7 |
| R1-Px7, R8-Px7, R16-Px7 | R3-Px7, R4-Px7, R9-Px7 | R4-Px7, R10-Px7, R16-Px7 | R7-Px7, R9-Px7, R10-Px7 |
| R1-Px7, R9-Px7, R10-Px7 | R3-Px7, R4-Px7, R10-Px7 | R4-Px7, R11-Px7, R12-Px7 | R7-Px7, R9-Px7, R11-Px7 |
| R1-Px7, R9-Px7, R11-Px7 | R3-Px7, R4-Px7, R11-Px7 | R4-Px7, R11-Px7, R13-Px7 | R7-Px7, R9-Px7, R12-Px7 |
| R1-Px7, R9-Px7, R12-Px7 | R3-Px7, R4-Px7, R12-Px7 | R4-Px7, R11-Px7, R14-Px7 | R7-Px7, R9-Px7, R13-Px7 |
| R1-Px7, R9-Px7, R13-Px7 | R3-Px7, R4-Px7, R13-Px7 | R4-Px7, R11-Px7, R15-Px7 | R7-Px7, R9-Px7, R14-Px7 |
| R1-Px7, R9-Px7, R14-Px7 | R3-Px7, R4-Px7, R14-Px7 | R4-Px7, R11-Px7, R16-Px7 | R7-Px7, R9-Px7, R15-Px7 |
| R1-Px7, R9-Px7, R15-Px7 | R3-Px7, R4-Px7, R15-Px7 | R4-Px7, R12-Px7, R13-Px7 | R7-Px7, R9-Px7, R16-Px7 |
| R1-Px7, R9-Px7, R16-Px7 | R3-Px7, R4-Px7, R16-Px7 | R4-Px7, R12-Px7, R14-Px7 | R7-Px7, R10-Px7, R11-Px7 |
| R1-Px7, R10-Px7, R11-Px7 | R3-Px7, R5-Px7, R6-Px7 | R4-Px7, R12-Px7, R15-Px7 | R7-Px7, R10-Px7, R12-Px7 |
| R1-Px7, R10-Px7, R12-Px7 | R3-Px7, R5-Px7, R7-Px7 | R4-Px7, R12-Px7, R16-Px7 | R7-Px7, R10-Px7, R13-Px7 |
| R1-Px7, R10-Px7, R13-Px7 | R3-Px7, R5-Px7, R8-Px7 | R4-Px7, R13-Px7, R14-Px7 | R7-Px7, R10-Px7, R14-Px7 |
| R1-Px7, R10-Px7, R14-Px7 | R3-Px7, R5-Px7, R9-Px7 | R4-Px7, R13-Px7, R15-Px7 | R7-Px7, R10-Px7, R15-Px7 |
| R1-Px7, R10-Px7, R15-Px7 | R3-Px7, R5-Px7, R10-Px7 | R4-Px7, R13-Px7, R16-Px7 | R7-Px7, R10-Px7, R16-Px7 |
| R1-Px7, R10-Px7, R16-Px7 | R3-Px7, R5-Px7, R11-Px7 | R4-Px7, R14-Px7, R15-Px7 | R7-Px7, R11-Px7, R12-Px7 |
| R1-Px7, R11-Px7, R12-Px7 | R3-Px7, R5-Px7, R12-Px7 | R4-Px7, R14-Px7, R16-Px7 | R7-Px7, R11-Px7, R13-Px7 |
| R1-Px7, R11-Px7, R13-Px7 | R3-Px7, R5-Px7, R13-Px7 | R4-Px7, R15-Px7, R16-Px7 | R7-Px7, R11-Px7, R14-Px7 |
| R1-Px7, R11-Px7, R14-Px7 | R3-Px7, R5-Px7, R14-Px7 | R5-Px7, R6-Px7, R7-Px7 | R7-Px7, R11-Px7, R15-Px7 |
| R1-Px7, R11-Px7, R15-Px7 | R3-Px7, R5-Px7, R15-Px7 | R5-Px7, R6-Px7, R8-Px7 | R7-Px7, R11-Px7, R16-Px7 |
| R1-Px7, R11-Px7, R16-Px7 | R3-Px7, R5-Px7, R16-Px7 | R5-Px7, R6-Px7, R9-Px7 | R7-Px7, R12-Px7, R13-Px7 |
| R1-Px7, R12-Px7, R13-Px7 | R3-Px7, R6-Px7, R7-Px7 | R5-Px7, R6-Px7, R10-Px7 | R7-Px7, R12-Px7, R14-Px7 |
| R1-Px7, R12-Px7, R14-Px7 | R3-Px7, R6-Px7, R8-Px7 | R5-Px7, R6-Px7, R11-Px7 | R7-Px7, R12-Px7, R15-Px7 |
| R1-Px7, R12-Px7, R15-Px7 | R3-Px7, R6-Px7, R9-Px7 | R5-Px7, R6-Px7, R12-Px7 | R7-Px7, R12-Px7, R16-Px7 |
| R1-Px7, R12-Px7, R16-Px7 | R3-Px7, R6-Px7, R10-Px7 | R5-Px7, R6-Px7, R13-Px7 | R7-Px7, R13-Px7, R14-Px7 |
| R1-Px7, R13-Px7, R14-Px7 | R3-Px7, R6-Px7, R11-Px7 | R5-Px7, R6-Px7, R14-Px7 | R7-Px7, R13-Px7, R15-Px7 |
| R1-Px7, R13-Px7, R15-Px7 | R3-Px7, R6-Px7, R12-Px7 | R5-Px7, R6-Px7, R15-Px7 | R7-Px7, R13-Px7, R16-Px7 |
| R1-Px7, R13-Px7, R16-Px7 | R3-Px7, R6-Px7, R13-Px7 | R5-Px7, R6-Px7, R16-Px7 | R7-Px7, R14-Px7, R15-Px7 |
| R1-Px7, R14-Px7, R15-Px7 | R3-Px7, R6-Px7, R14-Px7 | R5-Px7, R7-Px7, R8-Px7 | R7-Px7, R14-Px7, R16-Px7 |
| R1-Px7, R14-Px7, R16-Px7 | R3-Px7, R6-Px7, R15-Px7 | R5-Px7, R7-Px7, R9-Px7 | R7-Px7, R15-Px7, R16-Px7 |
| R1-Px7, R15-Px7, R16-Px7 | R3-Px7, R6-Px7, R16-Px7 | R5-Px7, R7-Px7, R10-Px7 | R8-Px7, R9-Px7, R10-Px7 |
| R2-Px7, R3-Px7, R4-Px7 | R3-Px7, R7-Px7, R8-Px7 | R5-Px7, R7-Px7, R11-Px7 | R8-Px7, R9-Px7, R11-Px7 |
| R2-Px7, R3-Px7, R5-Px7 | R3-Px7, R7-Px7, R9-Px7 | R5-Px7, R7-Px7, R12-Px7 | R8-Px7, R9-Px7, R12-Px7 |
| R2-Px7, R3-Px7, R6-Px7 | R3-Px7, R7-Px7, R10-Px7 | R5-Px7, R7-Px7, R13-Px7 | R8-Px7, R9-Px7, R13-Px7 |
| R2-Px7, R3-Px7, R7-Px7 | R3-Px7, R7-Px7, R11-Px7 | R5-Px7, R7-Px7, R14-Px7 | R8-Px7, R9-Px7, R14-Px7 |
| R2-Px7, R3-Px7, R8-Px7 | R3-Px7, R7-Px7, R12-Px7 | R5-Px7, R7-Px7, R15-Px7 | R8-Px7, R9-Px7, R15-Px7 |
| R2-Px7, R3-Px7, R9-Px7 | R3-Px7, R7-Px7, R13-Px7 | R5-Px7, R7-Px7, R16-Px7 | R8-Px7, R9-Px7, R16-Px7 |
| R2-Px7, R3-Px7, R10-Px7 | R3-Px7, R7-Px7, R14-Px7 | R5-Px7, R8-Px7, R9-Px7 | R8-Px7, R10-Px7, R11-Px7 |
| R2-Px7, R3-Px7, R11-Px7 | R3-Px7, R7-Px7, R15-Px7 | R5-Px7, R8-Px7, R10-Px7 | R8-Px7, R10-Px7, R12-Px7 |
| R2-Px7, R3-Px7, R12-Px7 | R3-Px7, R7-Px7, R16-Px7 | R5-Px7, R8-Px7, R11-Px7 | R8-Px7, R10-Px7, R13-Px7 |
| R2-Px7, R3-Px7, R13-Px7 | R3-Px7, R8-Px7, R9-Px7 | R5-Px7, R8-Px7, R12-Px7 | R8-Px7, R10-Px7, R14-Px7 |
| R2-Px7, R3-Px7, R14-Px7 | R3-Px7, R8-Px7, R10-Px7 | R5-Px7, R8-Px7, R13-Px7 | R8-Px7, R10-Px7, R15-Px7 |
| R2-Px7, R3-Px7, R15-Px7 | R3-Px7, R8-Px7, R11-Px7 | R5-Px7, R8-Px7, R14-Px7 | R8-Px7, R10-Px7, R16-Px7 |
| R2-Px7, R3-Px7, R16-Px7 | R3-Px7, R8-Px7, R12-Px7 | R5-Px7, R8-Px7, R15-Px7 | R8-Px7, R11-Px7, R12-Px7 |
| R2-Px7, R4-Px7, R5-Px7 | R3-Px7, R8-Px7, R13-Px7 | R5-Px7, R8-Px7, R16-Px7 | R8-Px7, R11-Px7, R13-Px7 |
| R2-Px7, R4-Px7, R6-Px7 | R3-Px7, R8-Px7, R14-Px7 | R5-Px7, R9-Px7, R10-Px7 | R8-Px7, R11-Px7, R14-Px7 |
| R2-Px7, R4-Px7, R7-Px7 | R3-Px7, R8-Px7, R15-Px7 | R5-Px7, R9-Px7, R11-Px7 | R8-Px7, R11-Px7, R15-Px7 |
| R2-Px7, R4-Px7, R8-Px7 | R3-Px7, R8-Px7, R16-Px7 | R5-Px7, R9-Px7, R12-Px7 | R8-Px7, R11-Px7, R16-Px7 |
| R2-Px7, R4-Px7, R9-Px7 | R3-Px7, R9-Px7, R10-Px7 | R5-Px7, R9-Px7, R13-Px7 | R8-Px7, R12-Px7, R13-Px7 |
| R2-Px7, R4-Px7, R10-Px7 | R3-Px7, R9-Px7, R11-Px7 | R5-Px7, R9-Px7, R14-Px7 | R8-Px7, R12-Px7, R14-Px7 |
| R8-Px7, R12-Px7, R15-Px7 | R9-Px7, R13-Px7, R16-Px7 | R10-Px7, R13-Px7, R14-Px7 | R11-Px7, R14-Px7, R15-Px7 |
| R8-Px7, R12-Px7, R16-Px7 | R9-Px7, R14-Px7, R15-Px7 | R10-Px7, R13-Px7, R15-Px7 | R11-Px7, R14-Px7, R16-Px7 |
| R8-Px7, R13-Px7, R14-Px7 | R9-Px7, R14-Px7, R16-Px7 | R10-Px7, R13-Px7, R16-Px7 | R11-Px7, R15-Px7, R16-Px7 |
| R8-Px7, R13-Px7, R15-Px7 | R9-Px7, R15-Px7, R16-Px7 | R10-Px7, R14-Px7, R15-Px7 | R12-Px7, R13-Px7, R14-Px7 |
| R8-Px7, R13-Px7, R16-Px7 | R10-Px7, R11-Px7, R12-Px7 | R10-Px7, R14-Px7, R16-Px7 | R12-Px7, R13-Px7, R15-Px7 |
| R8-Px7, R14-Px7, R15-Px7 | R10-Px7, R11-Px7, R13-Px7 | R10-Px7, R15-Px7, R16-Px7 | R12-Px7, R13-Px7, R16-Px7 |
| R8-Px7, R14-Px7, R16-Px7 | R10-Px7, R11-Px7, R14-Px7 | R11-Px7, R12-Px7, R13-Px7 | R12-Px7, R14-Px7, R15-Px7 |
| R8-Px7, R15-Px7, R16-Px7 | R10-Px7, R11-Px7, R15-Px7 | R11-Px7, R12-Px7, R14-Px7 | R12-Px7, R14-Px7, R16-Px7 |
| R9-Px7, R10-Px7, R11-Px7 | R10-Px7, R11-Px7, R16-Px7 | R11-Px7, R12-Px7, R15-Px7 | R12-Px7, R15-Px7, R16-Px7 |
| R9-Px7, R10-Px7, R12-Px7 | R10-Px7, R12-Px7, R13-Px7 | R11-Px7, R12-Px7, R16-Px7 | R13-Px7, R14-Px7, R15-Px7 |
| R9-Px7, R10-Px7, R13-Px7 | R10-Px7, R12-Px7, R14-Px7 | R11-Px7, R13-Px7, R14-Px7 | R13-Px7, R14-Px7, R16-Px7 |
| R9-Px7, R10-Px7, R14-Px7 | R10-Px7, R12-Px7, R15-Px7 | R11-Px7, R13-Px7, R15-Px7 | R13-Px7, R15-Px7, R16-Px7 |
| R9-Px7, R10-Px7, R15-Px7 | R10-Px7, R12-Px7, R16-Px7 | R11-Px7, R13-Px7, R16-Px7 | R14-Px7, R15-Px7, R16-Px7 |
| R9-Px7, R10-Px7, R16-Px7 | R9-Px7, R11-Px7, R14-Px7 | R9-Px7, R12-Px7, R13-Px7 | R9-Px7, R12-Px7, R16-Px7 |
| R9-Px7, R11-Px7, R12-Px7 | R9-Px7, R11-Px7, R15-Px7 | R9-Px7, R12-Px7, R14-Px7 | R9-Px7, R13-Px7, R14-Px7 |
| R9-Px7, R11-Px7, R13-Px7 | R9-Px7, R11-Px7, R16-Px7 | R9-Px7, R12-Px7, R15-Px7 | R9-Px7, R13-Px7, R15-Px7 |

In some embodiments, a MuSC-RC is a Px-MuSC-RC and comprises at least 2, or 3, or 4, or 5, or 6 REs from the Px gene (i.e., the MuSC does not comprise a RE from the MyoD1 gene). In some embodiments, the RE's in a Px-MuSC-RC can all be the same. For example, a Px-MuSC-RC useful in the methods and compositions as disclosed herein can comprise, e.g., a two R5-Px7 REs, or three R5-Px7 REs, or four R5-Px7 REs, etc. It is envisioned that all the RE's in a Px-MuSC-RC can be the same and can all be selected from any of R1-Px7 to R16-Px16.

In some embodiments, an exemplary Px-MuSC-RC useful in the methods and compositions as disclosed herein can comprise, e.g., two Px7 REs, where at least one RE is R5-Px7. In some embodiments, an exemplary Px-MuSC-RC useful in the methods and compositions as disclosed herein can comprise, e.g., two Px7 REs, where at least one RE is R8-Px7. In some embodiments, an exemplary Px-MuSC-RC useful in the methods and compositions as disclosed herein can comprise, e.g., two Px7 REs, where at least one RE is R5-Px7, and at least one Px7 RE is R8-Px7.

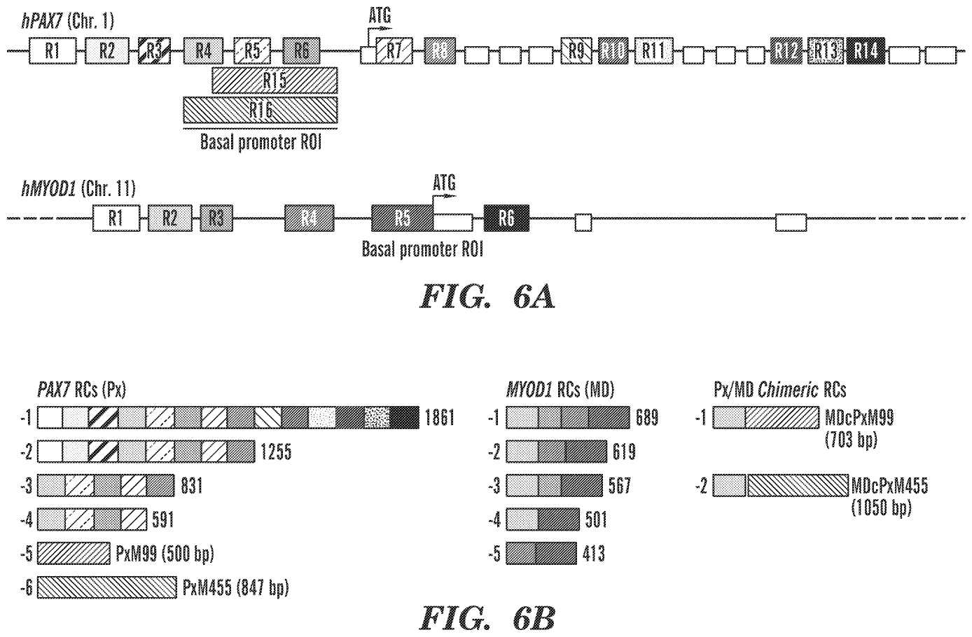

Exemplary MuSC-RC's comprising 2 or more Px7 RE's are shown in FIG. 6B and disclosed in the Examples. For illustrative purposes, an exemplary MuSC-RC is referred to as Px-1 (also referred to as “Px-1861”), and comprise 14 Pax7 RE's: R1-Px7 to R14-Px7 and corresponds to SEQ ID NO: 24. Another exemplary MuSC-RC is referred to as Px-2 (also referred to as “Px-1255”), and comprises eight RE's: R1-Px7, R2-Px7, R3-Px7, R4-Px7, R4-Px7, R5-Px7, R6-Px7, R7-Px7 and R8-Px7 and corresponds to SEQ ID NO: 25. Another exemplary MuSC-RC is referred to as Px-3 (also referred to as “Px-831”), and comprises five RE's: R4-Rx7, R5-Px7, R6-Px7, R7-Px7 and R8-Px7 and corresponds to SEQ ID NO: 26. Another exemplary MuSC-RC is referred to as Px-4 (also referred to as “Px-591”), and comprises four RE's: R4-Rx7, R5-Px7, R6-Px7 and R7-Px7 and corresponds to SEQ ID NO: 27. These Px7 MuSC-RC's (also shown in FIG. 6B) Px-1 to Px-4 (also referred to as Px-1861, Px-1255, Px-831, Px-591 respectively) are exemplary MuSC-RC's comprising 2 or more Pax7 RE's, and other combinations of two or more Pax7 RE's can be used, such as the combinations 2 or more Pax7 REs disclosed in Tables 1A or 1B.

In some embodiments, an exemplary Px-MuSC-RC useful in the methods and compositions as disclosed herein is Px831 (also referred to as Px-3) and corresponds to SEQ ID NO: 26. In some embodiments, an exemplary Px-MuSC-RC useful in the methods and compositions as disclosed herein is Px591 (also referred to as Px-4) and corresponds to SEQ ID NO: 27.

A MuSC-RC comprising one or more Pax7 REs can comprise at least two RE's from Pax7 gene as disclosed herein. In some embodiments, if one or more of the Pax7 RE's is modified or is a fragment from the reference Pax7 RE, it still retains the ability to bind a transcription factor that binds to the initial Pax7 RE from which it is derived. Representative TSBS for transcription factors for Pax7 RE's are shown in Table 4.

TABLE 4
Representative Transcription factor binding sites (TFBS) in each RE for Pax7 (R1-Px7 to
R14-Px7):

| | | | DNA sequence targeted by the TF binding domain |
|---|---|---|---|
| | | | (bold). All sequences are in 5′ to 3′ direction. |
| | | | Bold Italics are the DNA binding sequences on |
| RE Pax7 | TF Binding | Consensus sequence of | the reverse (antisense strand), written in 5′ to |
| region | domain | TFBS | 3′)that are bound by the TF) |
| R1-Px7 | CREB | Nnygcagtngnn | Gatgcagtggta (SEQ ID NO: 135) |
| | Pax7 | Nnnntaatyrattann | Acaataatcaattaca (SEQ ID NO: 136) |
| | binding | (SEQ ID NO: 48) | Aataatcaattacaat (SEQ ID NO: 137) |
| | | | Attgtaattgattatt (SEQ ID NO: 138) |
| | Pax7 only | taatyratta (SEQ ID | Taatcaatta (SEQ ID NO: 139) |
| | binding | NO: 60) | |
| | TCF/Lef1 | Nncctttgnn | Tgcctttgca (SEQ ID NO: 140) |
| | (Myf5 | | |
| | embryo | | |
| | paper) | | |
| | MCK | Nccagctgtcen (SEQ ID | Accagctgtcca (SEQ ID NO: 141) |
| | promoter | NO: 50) | |
| | Ebox, binds | | |
| | MyoD1 | | |
| | AP4 binding | Wgarycagctgyggncnk | Atgcccacaataatcaat (SEQ ID NO: 142) |
| | | (SEQ ID NO: 47) | Attgattattgtgggcat (SEQ ID NO: 143) |
| | AP-1/c-Fos | Nnnactgagtnn | Gaactcagtctt (SEQ ID NO: 144) |
| | | | Aagactgagttc (SEQ ID NO: 145) |
| | Dix/Hox | Nnccaattann | Gctaattggag (SEQ ID NO: 146) |
| | (Myf5 | | Ctccaattagc (SEQ ID NO: 147) |
| | embryo, with | | |
| | Fast-smad) | | |
| | Nkx2.5 | Nnngttaattnn | Ccaattaagggc (SEQ ID NO: 148) |
| | | | Gcccttaattgg (SEQ ID NO: 149) |
| R2-Px7 | Mef3 | Nnaacctgan | Ccaacctgac (SEQ ID NO: 150) |
| | Nkx2.5 | Nnngttaattnn | Gaggttaattcc (SEQ ID NO: 151) |
| | SP1 | Nnncccgccnnn | Ccacccacctct (SEQ ID NO: 152) |
| | TATA box | Nnnnnntataaannn | ttgtcatgtaaagCG (SEQ ID NO: 153) |
| | new | | |
| | Gli1 | Ntgggtggn | Accacccac |
| | | | Gtgggtggt |
| P3-Px7 | Ebox new | Nnncacttgnnn | Ctgcaaggggca (SEQ ID NO: 156) |
| | | | tgccccttgcag (SEQ ID NO: 157) |
| | TCF/Lef1 | Nncctttgnn | Ggcaaaggtc (SEQ ID NO: 158) |
| | (Myf5 | | Gacctttgcc (SEQ ID NO: 159) |
| | embryo | | Ggcgaagggg (SEQ ID NO: 160) |
| | paper) | | Ccccttcgcc (SEQ ID NO: 161) |
| | AP4 binding | Wgarycagctgyggncnk | Aaggtcacaggtgaggcg (SEQ ID NO: 162) |
| | | (SEQ ID NO: 47) | Cgcctcacctgtgacctt (SEQ ID NO: 163) |
| | | | Gcgggcag |
| | SP1 | Nnncccgccnnn | Aggggagggatg (SEQ ID NO: 164) |
| | | | Catccctcccct (SEQ ID NO: 165) |
| | | | Gagggcgggcag (SEQ ID NO: 166) |
| | | | Ctgcccgccctc (SEQ ID NO: 167) |
| | TATA box | Nnnnnntataaannn | ccctTTTTAAcagcc (SEQ ID NO: 168) |
| | new | | ggctgttaaaaaggg (SEQ ID NO: 169) |
| | CREB | Nnygcagtngnn | Ccccactgccga (SEQ ID NO: 170) |
| | | | Tcggcagtgggg (SEQ ID NO: 171) |
| P3-Px7 | region of R3- | comprises the | Catgctgcaaggggcaaaggtcacaggtgaggc |
| | Px7 of | following bindings | gaaggggagggatg (SEQ ID NO: 172) |
| | relevance: | sites for: ebox new, | |
| | | Ebox2 new, SP1, AP4 | |
| | | (2 sites), TCF/Lefl (2- | |
| | | sites) | |
| | region of R3- | comprises the | Gccgagagaggggggcag (SEQ ID NO: 38) |
| | Px7 of | following bindings | |
| | relevance: | sites for: AP4, CREB, | |
| | | SP1 | |
| P4-Px7 | AP4 binding | Wgarycagctgyggncnk | Agcagccgt |
| | | (SEQ ID NO: 47) | |
| | MyoD | | Gcgacctggg (SEQ ID NO: 175) |
| | Pax7 | | AATTAAa |
| | NF-1 | | Agttggccccctcccctt (SEQ ID NO: 177) |
| | AP2 | | Ctcccccagccc (SEQ ID NO: 178) |
| | SP1 | Nnncccgccnnn | Gccccctcccct (SEQ ID NO: 179) |
| | | | Cccccagccctt (SEQ ID NO: 180) |
| P5-Px7 | MyoD/AP4 | | Cccagctgat (SEQ ID NO: 181) |
| | Ebox new | Nuncacttgnnn | Gatcactcgcgc (SEQ ID NO: 182) |
| | SP1 | Nnncccgccnnn | Actcgcgccccc (SEQ ID NO: 183) |
| | | | Aaccccgtcacc (SEQ ID NO: 184) |
| | AP2 | | Cgccccctcgct (SEQ ID NO: 185) |
| | CREB | | Cgtca |
| | Ebox 2 new | Nnncacctgnn | Caccccctgtc (SEQ ID NO: 187) |
| | GC-Rich | Cttttccatttctctttcc | Cttttccatttctctttcc (SEQ ID NO: 188) |
| | transcription | (SEQ ID NO: 61) | |
| | binding (no | | |
| | TATA box) | | |
| P6-Px7 | Runx | | Gcgccagagcg (SEQ ID NO: 189) |
| | NF-1;AP2 | | Ctttggccccgagc (SEQ ID NO: 190) |
| | FKH | | Tatataaataaata (SEQ ID NO: 191) |
| | | | Gtgtttgtttgtttg (SEQ ID NO: 192) |
| | Oct | | Aaatatataaata (SEQ ID NO: 193) |
| | Pax | | Cgccagagcgcgagagcgc (SEQ ID NO: 194) |
| | SP1 | Nnncccgccnnn | Gagcgcgggagt (SEQ ID NO: 195) |
| | | | Actcccgagctc (SEQ ID NO: 196) |
| | | | Caccccacctca (SEQ ID NO: 197) |
| | | | aggggtgggggT (SEQ ID NO: 198) |
| | | | acccccacccct (SEQ ID NO: 199) |
| | | | gggggTgggggt(SEQ ID NO: 200) |
| | | | acccccacccc (SEQ ID NO: 201) |
| | | | tgtggaggggag (SEQ ID NO: 202) |
| | | | ctcccctccaca (SEQ ID NO: 203) |
| | | | aggggagggaga (SEQ ID NO: 204) |
| | | | tctccctcccct (SEQ ID NO: 205) |
| | | | gcggacgggaag (SEQ ID NO: 206) |
| | | | cttcccgtccgc (SEQ ID NO: 207) |
| | | | gtccccggcgtg (SEQ ID NO: 208) |
| | | | cttcccggcacg (SEQ ID NO: 209) |
| | | | aatggcgg |
| | | | ccgccatt |
| | | | cccggcgtgcgc (SEQ ID NO: 212) |
| | | | gcgcacgccggg (SEQ ID NO: 213) |
| | TATA box | Nnnnnntataaannn | ccagcttAtAATcgcg (SEQ ID NO: 214) |
| | new | | |
| | AP4 binding | Wgarycagctgyggncnk | Ctgcagccag (SEQ ID NO: 215) |
| | | (SEQ ID NO: 47) | Gcggacgg |
| | | | Gcggccct |
| | CREB | Nnygcagtngnn | Atcgcagcaggg (SEQ ID NO: 218) |
| | region of R6- | comprises the | Gtccccggcgtgcgcaagaatggcggcccttcccggcacgg |
| | Px7 of | following bindings | (SEQ ID NO: 219) |
| | relevance: | sites for: SP1, AP4 | |
| P7-Px7 | E2F | | Aaagagggaaaaa (SEQ ID NO: 220) |
| | Oct | | Aaaaaaatattc (SEQ ID NO: 221) |
| | Egr-1 | | Attcgttggcga (SEQ ID NO: 222) |
| | Oct-1/NF-1 | | Aaaaatattcgttggcgatttcttttcg (SEQ ID NO: 223) |
| | NF-1 | | Ccctggcccccttccct (SEQ ID NO: 224) |
| | TATA-like | | Gaataaaagaaaa (SEQ ID NO: 225) |
| | motif | | |
| | AP4 binding | Wgarycagctgyggncnk | Tgcgccctg |
| | | (SEQ ID NO: 47) | |
| | Ebox 2 new | Nnncacctgnn | Gctcccttgcgc (SEQ ID NO: 227) |
| | SP1 | Nnncccgccnnn | Cttccctcctca (SEQ ID NO: 228) |
| | TATA box | Nnnnnntataaannn | ctcctGTTTAAACga (SEQ ID NO: 229) |
| | new | | |
| P8-Px7 | AP4 binding | Wgarycagctgyggncnk | Tgcgcgccg |
| | | (SEQ ID NO: 47) | |
| | SP1 | Nnncccgccnnn | Gtgcgcgccggc (SEQ ID NO: 231) |
| | | | Gcagggggaga (SEQ ID NO: 232) |
| | | | Tctcccgcctgc (SEQ ID NO: 233) |
| | CREB | Nnygcagtngnn | Atcgcagcaggg (SEQ ID NO: 234) |
| | | | Tttgcagtggca (SEQ ID NO: 235) |
| | | | Atcaagtgcgcg (SEQ ID NO: 236) |
| | | | Cgcgcacttgat (SEQ ID NO: 237) |
| | Ebox new | Nnncacttgnnn | Gatcaagtgcgc (SEQ ID NO: 238) |
| | | | Gcgcacttgatc (SEQ ID NO: 239) |
| | | | Tcaagtgcgc (SEQ ID NO: 240) |
| | | | Gcgcacttga (SEQ ID NO: 241) |
| | Ebox 2 new | Nnncacctgnn | Cgcaggcggga (SEQ ID NO: 242) |
| | | | Tcccgcctgcg (SEQ ID NO: 243) |
| | region of R8- | comprises the | Gatcaagtgcgcgccggcagccacgcaggcgggaga (SEQ |
| | Px7 of | following bindings | ID NO: 244) |
| | relevance: | sites for: TATA box | |
| | | new, ebox new, CREB, | |
| | | Ebox2 new, SP1, AP4 | |
| | | binding | |
| P9-Px7 | CREB | Nnygcagtngnn | Atcgcagtgggg (SEQ ID NO: 245) |
| | Runx | nnRccacagnn | Ctctgtggcgt (SEQ ID NO: 246) |
| | | | Acgccacagag (SEQ ID NO: 247) |
| | SP1 | Nnncccgccnnn | tgtggcgtgcAA (SEQ ID NO: 248) |
| | | | TTgcacgccaca (SEQ ID NO: 249) |
| | Ebox new | Nnncacttgnnn | Ggccaagttccc (SEQ ID NO: 250) |
| | | | Gggaacttggcc (SEQ ID NO: 251) |
| | Ebox 2 new | Nnncacctgnn | Cccagatgccc (SEQ ID NO: 252) |
| | | | Gggcatctggg (SEQ ID NO: 253) |
| P10-Px7 | Ebox new | Nnncacttgnnn | Gctgcacttgg (SEQ ID NO: 254) |
| | CREB | Nnygcagtngnn | Gctgcacttgga (SEQ ID NO: 255) |
| | | | Gtcggctgcact (SEQ ID NO: 39) |
| | | | Agtgcagccgac (SEQ ID NO: 42) |
| | TCF/Lef1 | Nncctttgnn | Ggcaaagggg (SEQ ID NO: 43) |
| | (Myf5 | | Cccctttgcc (SEQ ID NO: 46) |
| | embryo | | Gccaaaggaa (SEQ ID NO: 49) |
| | paper) | | Ttcctttggc (SEQ ID NO: 51) |
| | SP1 | Nnncccgccnnn | Ggaggcgggggc (SEQ ID NO: 52) |
| | | | Gcccccgcctcc (SEQ ID NO: 53) |
| | | | Tggggaggggat (SEQ ID NO: 54) |
| | | | atcccctcccca (SEQ ID NO: 37) |
| | | | aagggagggggt (SEQ ID NO: 256) |
| | | | accccctccctt (SEQ ID NO: 257) |
| P11-Px7 | Mef3 | Nnaacctgan | Ggaacctgat (SEQ ID NO: 258) |
| | TATA box | Nnnnnntataaannn | Ggaccaataaaggc (SEQ ID NO: 259) |
| | new | | Ttgtttataatttac (SEQ ID NO: 260) |
| | | | Gtaaattataaacaa (SEQ ID NO: 261) |
| | TCF/Lef1 | Nncctttgnn | Tgcaaaggca (SEQ ID NO: 262) |
| | (Myf5 | | Tgcctttgca (SEQ ID NO: 263) |
| | embryo | | |
| | paper) | | |
| | SP1 | Nnncccgccnnn | Aaaggcagggct (SEQ ID NO: 264) |
| | | | Agccctgccttt (SEQ ID NO: 265) |
| | | | Cagggctggaga (SEQ ID NO: 266) |
| | | | tctccagccctg (SEQ ID NO: 267) |
| P12-Px7 | MyoD1 | GCAGCTGTCNCT | tgcagctgtctct (SEQ ID NO: 268) |
| | binding site | (SEQ ID NO: 44) | |
| | (jasper) | | |
| | TCF/Lef1 | Nncctttgnn | cacctttgca (SEQ ID NO: 269) |
| | (Myf5 | | |
| | embryo | | |
| | paper) | | |
| | mdMyoD1 | | Tgcagctgtctct (SEQ ID NO: 63) |
| | binding | | |
| | perfect | | |
| P13-Px7 | SP1 | Nnncccgccnnn | Cctcccgcctgc (SEQ ID NO: 270) |
| | | | Ccgcctgcccct (SEQ ID NO: 271) |
| | | | Tgcccctcctcc (SEQ ID NO: 272) |
| | Dix/Hox | Nnccaattann | ggccaattagg (SEQ ID NO: 273) |
| | (Myf5 | | |
| | embryo, with | | |
| | Fast-smad) | | |
| | Ebox 2 new | Nnncacctgnn | Aggcccctgac (SEQ ID NO: 274) |
| | | | Tgacacctgga (SEQ ID NO: 275) |
| | | | tgacacctggag (SEQ ID NO: 276) |
| | Ebox new | Nnncacttgnnn | Tgacacctggag (SEQ ID NO: 277) |
| | | | Tgtcacttgc (SEQ ID NO: 278) |
| | | | tgtcacttgctc (SEQ ID NO: 279) |
| | CREB | Nnygcagtngnn | ccgcagaagCT (SEQ ID NO: 280) |
| | AP4 binding | Wgarycagctgyggncnk | Agggccgcag (SEQ ID NO: 281) |
| | | (SEQ ID NO: 47) | ctgcggccct (SEQ ID NO: 282) |
| | Region of | comprises the | cctcccgcctgcccctcctcccctcagctcggccaattaggcccct |
| | R13-Px7 of | following bindings | gacacctgga (SEQ ID NO: 283) |
| | relevance: | sites for: SP1(3-sites), | |
| | | Dix/Hox, Ebox new | |
| | | and Ebox 2 new)(x2) | |
| P14-Px7 | Myogenin | NNNNNARCAGCTG | Tcccaggcac (SEQ ID NO: 284) |
| | | YN | Ccccaggcag (SEQ ID NO: 285) |
| | | | Atccaggcac (SEQ ID NO: 286) |
| | | | atccaggcac (SEQ ID NO: 287) |
| | SP1 | Nnncccgccnnn | gtcCCAgccaaa (SEQ ID NO: 288) |
| | Ebox new | Nnncacttgnnn | Cgccaagt |
| | | | acttggcg |
| | AP4 binding | Wgarycagctgyggncnk | Atggccaca |
| | | (SEQ ID NO: 47) | tgtggccat |

### (b) MyoD1-MuSC-RC (MD-MuSC-RC):

In some embodiments, a MuSC-RC is a MD-MuSC-RC and comprises at least two REs from the MyoD1 gene (i.e., the MuSC does not comprise a RE from the Pax7 gene). Exemplary combinations of 2 REs from MyoD 1 gene include, but are not limited to, the combinations shown in Table 2A.

TABLE 2A
Exemplary combinations of at least 2 RE selected from any of the 7 MyoD1
REs. These can be in any order, and not necessarily in the order shown.
Table 2A: at least 2 RE selected from any of the 6 MyoD1 RE (21 options) (R1-MD
to R6-MD); (which can be in any order, and not necessarily in the order shown)

| | | | | |
|---|---|---|---|---|
| R1-MD, R2-MD | R1-MD, R5-MD | R2-MD, R4-MD | R3-MD, R4-MD | R4-MD, R5-MD |
| R1-MD, R3-MD | R1-MD, R6-MD | R2-MD, R5-MD | R3-MD, R5-MD | R4-MD, R6-MD |
| R1-MD, R4-MD | R2-MD, R3-MD | R2-MD, R6-MD | R3-MD, R6-MD | R5-MD, R6-MD |

In some embodiments, a MuSC-RC is a MD-MuSC-RC and comprises at least three REs from the MyoD gene (i.e., the MuSC does not comprise a RE from the Pax7 gene). Exemplary combinations of 3 REs from MyoD include, but are not limited to, the combinations shown in Table 2B.

TABLE 2B
Exemplary combinations of a at least 3 RE selected from any of the 7 MyoD
REs. These can be in any order, and not necessarily in the order shown.
Table 2B: Exemplary MD-MuSC-RC comprising a combination of at least 3 RE selected
from any of the six MyoD1 RE (R1-MD to R6-MD). (which can be in any order, and
not necessarily in the order shown)

| | | | |
|---|---|---|---|
| R1-MD, R2-MD, R3-MD | R1-MD, R3-MD, R5-MD | R2-MD, R3-MD, R4-MD | R2-MD, R5-MD, R6-MD |
| R1-MD, R2-MD, R4-MD | R1-MD, R3-MD, R6-MD | R2-MD, R3-MD, R5-MD | R3-MD, R4-MD, R5-MD |
| R1-MD, R2-MD, R5-MD | R1-MD, R4-MD, R5-MD | R2-MD, R3-MD, R6-MD | R3-MD, R4-MD, R6-MD |
| R1-MD, R2-MD, R6-MD | R1-MD, R4-MD, R6-MD | R2-MD, R4-MD, R5-MD | R3-MD, R5-MD, R6-MD |
| R1-MD, R3-MD, R4-MD | R1-MD, R5-MD, R6-MD | R2-MD, R4-MD, R6-MD | R4-MD, R5-MD, R6-MD |

In some embodiments, a MuSC-RC is a MD-MuSC-RC and comprises at least four REs from the MyoD gene (i.e., the MuSC does not comprise a RE from the Pax7 gene). Exemplary combinations of 4 REs from MyoD include, but are not limited to, the combinations shown in Table 2C.

TABLE 2C
Exemplary combinations of at least 4 RE selected from any of the 6 MyOD
REs. These can be in any order, and not necessarily in the order shown.
Table 2C: Exemplary MD-MuSC-RC comprising at least 4 RE selected from any of the
6 MyoD1 RE (which can be in any order, and not necessarily in the order shown)

| | | | |
|---|---|---|---|
| R1-MD, R2-MD, R3-MD, R4-MD | R1-MD, R2-MD, R4-MD, R6-MD | R1-MD, R3-MD, R4-MD, R6-MD | R2-MD, R3-MD, R4-MD, R6-MD |
| R1-MD, R2-MD, R3-MD, R5-MD | R1-MD, R2-MD, R5-MD, R6-MD | R1-MD, R3-MD, R5-MD, R6-MD | R2-MD, R3-MD, R5-MD, R6-MD |
| R1-MD, R2-MD, R3-MD, R6-MD | R1-MD, R3-MD, R4-MD, R5-MD | R1-MD, R4-MD, R5-MD, R6-MD | R2-MD, R4-MD, R5-MD, R6-MD |
| R1-MD, R2-MD, R4-MD, R5-MD | | R2-MD, R3-MD, R4-MD, R5-MD | R3-MD, R4-MD, R5-MD, R6-MD |

In some embodiments, a MuSC-RC is a MD-MuSC-RC and comprises at least five REs from the MyoD gene (i.e., the MuSC does not comprise a RE from the Pax7 gene). Exemplary combinations of 5 REs from MyoD include, but are not limited to, the combinations shown in Table 2D.

TABLE 2D
Exemplary combinations of at least 4 RE selected from any of the 6 MyoD
REs. These can be in any order, and not necessarily in the order shown.
Table 2D: Exemplary MD-MuSC-RC comprising a combination of at least 5
RE selected from any of the 6 MyoD1 RE (selected from R1-MD to R6-MD).
(which can be in any order, and not necessarily in the order shown)

| | |
|---|---|
| R1-MD, R2-MD, R3-MD, R4-MD, R5-MD | R1-MD, R2-MD, R4-MD, R5-MD, R6-MD |
| R1-MD, R2-MD, R3-MD, R4-MD, R6-MD | R1-MD, R3-MD, R4-MD, R5-MD, R6-MD |
| R1-MD, R2-MD, R3-MD, R5-MD, R6-MD | R2-MD, R3-MD, R4-MD, R5-MD, R6-MD |

In some embodiments, a MuSC-RC is a MD-MuSC-RC and comprises at least 2, or 3, or 4, or 5, or 6 REs from the MyoD1 gene (i.e., the MuSC does not comprise a RE from the Pax7 gene). In some embodiments, the RE's in a MD-MuSC-RC can all be the same. For example, a MD-MuSC-RC useful in the methods and compositions as disclosed herein can comprise, e.g., two R2-MD REs, or three R2-MD REs, or four R2-MD REs, etc. It is envisioned that all the RE's in a MD-MuSC-RC can be the same and can all be selected from any of: R1-MD, R2-MD, R3-MD, R4-MD, R5-MD and R6-MD. In some embodiments, an exemplary MD-MuSC-RC useful in the methods and compositions as disclosed herein can comprise, e.g., two MD REs, where at least one RE is R2-MD. In some embodiments, an exemplary MD-MuSC-RC useful in the methods and compositions as disclosed herein can comprise, e.g., two MD REs, where at least one RE is R5-MD. In some embodiments, an exemplary MD-MuSC-RC useful in the methods and compositions as disclosed herein can comprise, e.g., two MD REs, where at least one RE is R5-MD, and at least one MD RE is R2-MD.

Exemplary MuSC-RC's comprising 2 or more REs from the MyoD1 gene are shown in FIG. 6B and disclosed in the Examples. For illustrative purposes, an exemplary MuSC-RC is referred to as MD-1 (also referred to as “MD-689”), and comprises four RE's: R2-MD, R3-MD, R4-MD and R5MD and corresponds to SEQ ID NO: 30. Another exemplary MuSC-RC is referred to as MD-2 (also referred to as “MD-619”), and comprises three RE's: R2-MD, R4-MD and R5-MD and corresponds to SEQ ID NO: 31. Another exemplary MuSC-RC is referred to as MD-3 (also referred to as “MD-597”), and comprises three RE's: R2-MD, R3-MD and R5-MD and corresponds to SEQ ID NO: 32. Another exemplary MuSC-RC is referred to as MD-4 (also referred to as “MD-501”), and comprises 2 RE's: R2-MD and R5-MD and corresponds to SEQ ID NO: 33. Another exemplary MuSC-RC is referred to as MD-5 (also referred to as “MD-413”), and comprises R4-MD and R5-MD and corresponds to SEQ ID NO: 34. These MD MuSC-RC's (also shown in FIG. 6B) MD-1 to MD-5 (also referred to as MD-689, MD-619, MD-567, MD-501, MD-413 respectively) are exemplary MuSC-RC's comprising 2 or more MyoD1 RE's, and other combinations of two or more MyoD1 RE's can be used, such as the combinations 2 or more MyoD1 REs disclosed in Tables 2A or 2B.

In some embodiments, an exemplary MD-MuSC-RC useful in the methods and compositions as disclosed herein comprises MD619 (also referred to as MD-2) and corresponds to SEQ ID NO: 31. In some embodiments, an exemplary MD-MuSC-RC useful in the methods and compositions as disclosed herein comprises MD689 (also referred to as MD-1) and corresponds to SEQ ID NO: 30.

MuSC-RC comprising one or more MyoD1 REs can comprise at least two RE's from MyoD1 gene as disclosed herein. In some embodiments, if one or more of the MyoD1 RE's is modified or is a fragment from the reference MyoD1 RE, it still retains the ability to bind a transcription factor that binds to the initial MyoD1 RE from which it is derived. Representative TSBS for transcription factors for MyoD1 RE's are shown in Table 5.

+0 TABLE 5
Representative Transcription factor binding sites (TFBS) in each RE for MyoD1 (R1-MD
to R6-MD):

| | | | DNA sequence targeted by the TF binding domain. |
|---|---|---|---|
| | | | All sequences are in 5′ to 3′ direction. |
| | | | Italics are the DNA binding |
| MD | TF Binding | Consensus sequence of | sequence on the reverse (antisense |
| region | domain | TFBS | strand), written in 5′ to 3′)) |
| R1-MD | MyoD1 | NSCASCTGYY | TGCACCTGCT (SEQ ID NO: 65) |
| | binding | | |
| | MyoD | SCASCTGYBN | GCACCTGCTG (SEQ ID NO: 66) |
| | binding | | |
| | Myogenin | NNNNNARCAGCTGYN | CACACCTGCTCCGGC (SEQ ID NO: 67) |
| | | | GCCGGAGCAGGTGTG (SEQ ID NO: 68) |
| | | | GACAGTTGCTGCATG(SEQ ID NO: 69) |
| | | | CATGCAGCAACTGTCT (SEQ ID NO: 70) |
| | Myf6/mr4- | NNNRRCARYTGYYN(S | GGAGACAGTTGCTG (SEQ ID NO: 71) |
| | directs diff. | EQ ID NO: 40) | ATGCAGCAACTGTCT (SEQ ID NO: 72) |
| | | | AGACAGTTGCTGCA (SEQ ID NO: 73) |
| | | | Tgcagcaactgtct (SEQ ID NO: 74) |
| | pDES- | GCCCTGCCTTTGTTCTC | GCCCTGCCTTTGTTCTCATG (SEQ ID NO: 75) |
| | RO1-R | ATG (SEQ ID NO: 45) | |
| R2- | MyoD1 | NSCASCTGYY | AGCAGCTGGG (SEQ ID NO: 76) |
| MD | binding | | CCCAGCYGCT (SEQ ID NO: 77) |
| (partial | Myf6/mr4- | NNNRRCARYTGYYN(S | CAGCAGCTGGTCAC (SEQ ID NO: 78) |
| core) | directs diff. | EQ ID NO: 40) | GTGACCAGCTGCTG (SEQ ID NO: 79) |
| | Myogenin | RRCAGSTGSNG (SEQ ID | AGCAGCTGGGG (SEQ ID NO: 80) |
| | binding | NO: 41) | |
| | (negative | | |
| | regulator) | | |
| | Myogenin | NNNNNARCAGCTGYN | AGCAGCTGGTCACAA (SEQ ID NO: 81) |
| | | | TTGTGACCAGCTGCT (SEQ ID NO: 82) |
| | AP-1 | | TGACTCA |
| | H4tf-1 | | GGGGGAGGG |
| | Nf-1 | | TGGCTGCCTCCAA (SEQ ID NO: 85) |
| R3-MD | MyoD1 | NSCASCTGYY | GGCAGCTGCT (SEQ ID NO: 86) |
| | binding | | TGCAGCTGCT (SEQ ID NO: 87) |
| | | | GGCAGCTGCT (SEQ ID NO: 88) |
| | | | AGCAGCTGCC (SEQ ID NO: 89) |
| | MyoD | SCASCTGYBN | GCAGCTGCTG (SEQ ID NO: 90) |
| | binding | | GCAGCTGCTT (SEQ ID NO: 91) |
| | | | GGGCAGCTGC (SEQ ID NO: 92) |
| | | | GCAGCTGCCC (SEQ ID NO: 93) |
| | Myogenin | NNNNNARCAGCTGYN | ACTGGGGCAGCTGCT (SEQ ID NO: 94) |
| | | | GGGGCTGCAGCTGCT (SEQ ID NO: 95) |
| | | | GGCAGCTGCTGGGGG (SEQ ID NO: 96) |
| | | | CCCCCAGCAGCTGCC (SEQ ID NO: 97) |
| | | | TGCAGCTGCTTAATC (SEQ ID NO: 98) |
| | | | Gattaagcagctgca (SEQ ID NO: 99) |
| | Myf6/mr4- | NNNRRCARYTGYYN | TGGGGCAGCTGCTG (SEQ ID NO: 100) |
| | directs diff. | (SEQ ID NO: 40) | GGCTGCAGCTGCTT (SEQ ID NO: 101) |
| | | | GGGCAGCTGCTGGG (SEQ ID NO: 102) |
| | | | CCCAGCAGCTGCCC (SEQ ID NO: 103) |
| | Myogenin | RRCAGSTGSNG (SEQ ID | GGCAGCTGCTG (SEQ ID NO: 104) |
| | binding | NO: 41) | CTGCAGCTGCT (SEQ ID NO: 105) |
| | (negative | | AGCAGCTGCAG (SEQ ID NO: 106) |
| | regulator) | | |
| | Myf5 | NNRCAGCTGY | GGGCAGCTGC (SEQ ID NO: 107) |
| | binding - | | CTGCAGCTGC (SEQ ID NO: 108) |
| | early | | GCAGCTGCTG (SEQ ID NO: 109) |
| | activation | | CAGCAGCTGC (SEQ ID NO: 110) |
| | with MyoD | | GCAGCTGCTT (SEQ ID NO: 111) |
| | | | AAGCAGCTGC (SEQ ID NO: 307) |
| | region of | comprises the following | ACTGGGGCAGCTGCTGGGGGCTGCAGCTGC |
| | R3-MD | binding sites for: MyoD1 | TTAATC (SEQ ID NO: 112) |
| | (partial core | binding, MyoD binding, | |
| | enhancer) | Myogenin, Myf6/mr4- | |
| | of | directs diff., Myogenin | |
| | relevance | binding (negative regulator, | |
| | | Myf5 binding - early | |
| | | activation with MyoD | |
| | Myf6/mrf4- | NNNRRCARYTGYYN(S | Binds to 3 regions in R3-MD: |
| | directed | EQ ID NO: 40) | TGGGGCAGCTGCTGGGGGCTGCAGCTGCTT |
| | differentiation | | (SEQ ID NO: 113) |
| | Myogenin | RRCAGSTGSNG (SEQ ID | 4 sites in R3-MD that are Myogenin |
| | | NO: 41) | binding (2F and 2R) |
| | | | GGGGCTGCAGCTGCTTAATC |
| | | | (SEQ ID NO: 114) |
| R4-MD | MyoD1 | NSCASCTGYY | GGCAGGTGCA (SEQ ID NO: 115) |
| | binding | | TGCACCTGCC (SEQ ID NO: 116) |
| | MyoD | SCASCTGYBN | GGGCAGGTGC (SEQ ID NO: 117) |
| | binding | | GCACCTGCCC (SEQ ID NO: 118) |
| | Myogenin | RRCAGSTGSNG (SEQ ID | GGCAGGTGCAG (SEQ ID NO: 119) |
| | binding | NO: 41) | |
| | (negative | | |
| | regulator) | | |
| | TATA Box | Nnnnnntataaannn | CCATTTATAGCCCCa (SEQ ID NO: 120) |
| | New | | tGGGGCTATAAATGG (SEQ ID NO: 121) |
| R5-MD | MyoD1 | NSCASCTGYY | CCCAGCTGCC (SEQ ID NO: 122) |
| | binding | | |
| | MyoD | SCASCTGYBN | CCAGCTGCCC (SEQ ID NO: 123) |
| | binding | | |
| | Myogenin | RRCAGSTGSNG (SEQ ID | CCCCAGCTGCC (SEQ ID NO: 124) |
| | binding | NO: 41) | GGCAGCTGGGG (SEQ ID NO: 125) |
| | (negative | | |
| | regulator) | | |
| R6-MD | Myf5 | NNRCAGCTGY | GGGCAGCTGT (SEQ ID NO: 126) |
| | binding - | | |
| | early | | |
| | activation | | |
| | with MyoD | | |
| | Myf6/mr4- | NNNRRCARYTGYYN | CTGGGCAGCTGTCA (SEQ ID NO: 127) |
| | directs diff. | (SEQ ID NO: 40) | GGGCAGCTGTCACT (SEQ ID NO: 128) |
| | | | AGTGGACAGCTGCCC (SEQ ID NO: 129) |
| | Myogenin | NNNNNARCAGCTGYN | TCCTGGGCAGCTGTC (SEQ ID NO: 130) |
| | | | GGCAGCTGTCACTGG (SEQ ID NO: 131) |
| | | | CCAGTGACAGCTGCC (SEQ ID NO: 132) |
| | MyoD1 | GCAGCTGTCNCT (SEQ | GCAGCTGTCACT (SEQ ID NO: 133) |
| | binding site | ID NO: 44) | TCCTGGGCAGCTGTCACTGG |
| | (Jaspar) | comprises the following | (SEQ ID NO: 134) |
| | region of | bindings sites for: | |
| | R6-MD of | Myf5 binding - early | |
| | relevance: | activation with MyoD, | |
| | | Myf6/mr4-directs diff | |
| | | (2;Forward and Reverse), | |
| | | MyoDI binding site | |
| | | (Jaspar, and Myogenin (2 | |
| | | sites; forward and R) | |
| | | Myogenin binding | |

### (C) Chimeric MuSC-RC ((Px/MD MuSC-RC):

In some embodiments, a MuSC-RC is a chimeric MuSC-RC-that is, it comprises at least one RE from the MyoD 1 gene, and at least one RE from the Pax7 gene. Exemplary combinations of at least one RE from the Pax7 gene (e.g., selected from any of R1-Px7 to R16-Px7) and at least one RE from the MyoD1 gene (e.g., selected from any of 2R-MD to R6-MD), include, but are not limited to, the combinations shown in Table 3. In some embodiments, any MuSC-RC disclosed in Table 3 can further comprise at least 1, 2, 3, 4, 5, 6, 7, 8, 9, 10 or more than 10 additional REs selected from any of R1-Px7 to R16-Px7 or R1-MD to R6-MD.

TABLE 3
Exemplary Px/MD chimeric MuSC-RC, showing exemplary combinations
of (i) at least one RE selected from any of the 7 MyoD1 REs, and
(ii) at least one RE selected from any of the 16 Pax7 REs. These
can be in any order, and not necessarily in the order shown.

| | R1-MD | R2-MD | R3-MD | R4-MD | R5-MD | R6-MD |
|---|---|---|---|---|---|---|
| R1-Px7 | R1-MD, | R2-MD, | R3-MD, | R4-MD, | R5-MD, | R6-MD, |
| | R1-Px7 | R1-Px7 | R1-Px7 | R1-Px7 | R1-Px7 | R1-Px7 |
| R2-Px7 | R1-MD, | R2-MD, | R3-MD, | R4-MD, | R5-MD, | R6-MD, |
| | R2-Px7 | R2-Px7 | R2-Px7 | R2-Px7 | R2-Px7 | R2-Px7 |
| R3-Px7 | R1-MD, | R2-MD, | R3-MD, | R4-MD, | R5-MD, | R6-MD, |
| | R3-Px7 | R3-Px7 | R3-Px7 | R3-Px7 | R3-Px7 | R3-Px7 |
| R4-Px7 | R1-MD, | R2-MD, | R3-MD, | R4-MD, | R5-MD, | R6-MD, |
| | R4-Px7 | R4-Px7 | R4-Px7 | R4-Px7 | R4-Px7 | R4-Px7 |
| R5-Px7 | R1-MD, | R2-MD, | R3-MD, | R4-MD, | R5-MD, | R6-MD, |
| | R5-Px7 | R5-Px7 | R5-Px7 | R5-Px7 | R5-Px7 | R5-Px7 |
| R6-Px7 | R1-MD, | R2-MD, | R3-MD, | R4-MD, | R5-MD, | R6-MD, |
| | R6-Px7 | R6-Px7 | R6-Px7 | R6-Px7 | R6-Px7 | R6-Px7 |
| R7-Px7 | R1-MD, | R2-MD, | R3-MD, | R4-MD, | R5-MD, | R6-MD, |
| | R7-Px7 | R7-Px7 | R7-Px7 | R7-Px7 | R7-Px7 | R7-Px7 |
| R8-Px7 | R1-MD, | R2-MD, | R3-MD, | R4-MD, | R5-MD, | R6-MD, |
| | R8-Px7 | R8-Px7 | R8-Px7 | R8-Px7 | R8-Px7 | R8-Px7 |
| R9-Px7 | R1-MD, | R2-MD, | R3-MD, | R4-MD, | R5-MD, | R6-MD, |
| | R9-Px7 | R9-Px7 | R9-Px7 | R9-Px7 | R9-Px7 | R9-Px7 |
| R10-Px7 | R1-MD, | R2-MD, | R3-MD, | R4-MD, | R5-MD, | R6-MD, |
| | R10-Px7 | R10-Px7 | R10-Px7 | R10-Px7 | R10-Px7 | R10-Px7 |
| R11-Px7 | R1-MD, | R2-MD, | R3-MD, | R4-MD, | R5-MD, | R6-MD, |
| | R11-Px7 | R11-Px7 | R11-Px7 | R11-Px7 | R11-Px7 | R11-Px7 |
| R12-Px7 | R1-MD, | R2-MD, | R3-MD, | R4-MD, | R5-MD, | R6-MD, |
| | R12-Px7 | R12-Px7 | R12-Px7 | R12-Px7 | R12-Px7 | R12-Px7 |
| R13-Px7 | R1-MD, | R2-MD, | R3-MD, | R4-MD, | R5-MD, | R6-MD, |
| | R13-Px7 | R13-Px7 | R13-Px7 | R13-Px7 | R13-Px7 | R13-Px7 |
| R14-Px7 | R1-MD, | R2-MD, | R3-MD, | R4-MD, | R5-MD, | R6-MD, |
| | R14-Px7 | R14-Px7 | R14-Px7 | R14-Px7 | R14-Px7 | R14-Px7 |
| R15-Px7 | R1-MD, | R2-MD, | R3-MD, | R4-MD, | R5-MD, | R6-MD, |
| | R15-Px7 | R15-Px7 | R15-Px7 | R15-Px7 | R15-Px7 | R15-Px7 |
| R16-Px7 | R1-MD, | R2-MD, | R3-MD, | R4-MD, | R5-MD, | R6-MD, |
| | R16-Px7 | R16-Px7 | R16-Px7 | R16-Px7 | R16-Px7 | R16-Px7 |

### (i) MDcPxM99:

In some embodiments, a Px/MD chimeric MuSC-RC useful in the methods and compositions as disclosed herein comprises at least (i) R2-MD and at least (ii) R15-Px7. Such a chimeric Px/MD chimeric MuSC-RC of MDcPxM99 comprises a R2-MD sequence according to SEQ ID NO: 18, or a functional variant or functional fragment thereof having a sequence that is at least 60%, 65%, 70%, 75%, 80%, 85%, 90%, 95%, 96%, 97%, 98%, or 99% identical thereto, and a R15-Px7 sequence of SEQ ID NO: 15 or a functional variant or functional fragment thereof having a sequence that is at least 60%, 65%, 70%, 75%, 80%, 85%, 90%, 95%, 96%, 97%, 98%, or 99% identical to SEQ ID NO: 18. In some embodiments, a sequence of SEQ ID NO: 18 and SEQ ID NO: 15 are joined by a spacer sequence a disclosed herein. In some embodiments, the order of the sequence can be, in a 5′- to 3′-order of: SEQ ID NO: 18 and SEQ ID NO: 15, or functional fragments or variants thereof as described herein. In alternative embodiments, the order of the sequence can be, in a 5′- to 3′-order of: SEQ ID NO: 15 and SEQ ID NO: 18, or functional fragments, or variants thereof as described herein.

A MDcPxM99 regulatory sequence can have a sequence according to SEQ ID NO: 34. Functional variants thereof may have a sequence that is at least 60%, 65%, 70%, 75%, 80%, 85%, 90%, 95%, 96%, 97%, 98%, or 99% identical thereto. In some embodiments, a functional variant of a MDcPxM99 MuSC-RC can have a sequence according to SEQ ID NO: 34 retain at least 80%, at least 90%, at least 95% or at least 100% of the activity of the reference cassette (i.e., SEQ ID NO: 34) in a reporter assay. In some embodiments, a functional variant of aMDcPxM99 MuSC-RC as described herein promotes expression in a muscle stem cell-specific manner to a similar level to that of SEQ ID NO: 34, and does not promote expression in non-muscle cells to a similar level to that of SEQ ID NO: 34.

Functional variants of a MDcPxM99 regulatory cassette as disclosed herein can have a sequence which varies from SEQ ID NO: 34, but which substantially retain activity as a muscle-stem cell-specific regulatory cassette. It will be appreciated by the skilled person that it is possible to vary the sequence of a RE while retaining its ability to bind to the requisite transcription factors (TFs) and promote or enhance expression. A functional variant can comprise substitutions, deletions and/or insertions compared to a reference RE, provided they do not render the RE substantially non-functional.

In some embodiments, a functional variant of a MDcPxM99 regulatory cassette as disclosed herein can be viewed as a RC which, when substituted in place of a MDcPxM99 RC, substantially retains its activity. For illustrative purposes only, a MDcPxM99 SCRC which comprises a functional variant of SEQ ID NO: 18 substituted in place of SEQ ID NO: 18 in a MDcPxM99 regulatory cassette as disclosed herein, preferably retains 80% of its activity, more preferably 90% of its activity, more preferably 95% of its activity, and yet more preferably 100% of its activity. Similarly, a MDcPxM99 SCRC which comprises a functional variant of SEQ ID NO: 15 substituted in place of SEQ ID NO: 15 in a MDcPxM99 regulatory cassette as disclosed herein, preferably retains 80% of its activity, more preferably 90% of its activity, more preferably 95% of its activity, and yet more preferably 100% of its activity.

As an illustrative example only, considering MDcPxM99 MuSC-RC (e.g., see exemplary top Px/MD MuSC-RC in FIG. 6B), R2-MD in MDcPxM99 can be replaced with a functional variant of R2-MD, and/or the R15-Px7 in MDcPxM99 can be replaced with a functional variant of R2-MD or R15-Px7, respectively, and the MDcPxM99 SCRC substantially retains its activity. Retention of activity can be assessed by comparing expression of a suitable reporter under the control of the reference promoter with an otherwise identical promoter comprising the substituted RE under equivalent conditions.

In some embodiments, the MDcPxM99 RC or a functional variant thereof, has a length of 703 or fewer nucleotides, e.g., 700, 600, 500, 400, 300, 200 or 150 or fewer nucleotides.

(ii) mDcPxM455:

In some embodiments, a Px/MD chimeric MuSC-RC useful in the methods and compositions as disclosed herein comprises R2-MD and R15-Px7. In some embodiments, a Px/MD chimeric MuSC-RC useful in the methods and compositions as disclosed herein comprises R2-MD and R16-Px7.

In some embodiments, a Px/MD chimeric MuSC-RC useful in the methods and compositions as disclosed herein comprises at least (i) R2-MD and at least (ii) R16-Px7. Such a chimeric Px/MD chimeric MuSC-RC of MDcPxM455 comprises a R2-MD sequence according to SEQ ID NO: 18, or a functional fragment thereof having a sequence that is at least 60%, 65%, 70%, 75%, 80%, 85%, 90%, 95%, 96%, 97%, 98%, or 99% identical thereto, and a R16-Px7 sequence of SEQ ID NO: 16 or a functional variant or functional fragment thereof having a sequence that is at least 60%, 65%, 70%, 75%, 80%, 85%, 90%, 95%, 96%, 97%, 98%, or 99% identical to SEQ ID NO: 18. In some embodiments, a sequence of SEQ ID NO: 18 and SEQ ID NO: 16 are joined by a spacer sequence as described herein. In some embodiments, the order of the sequence can be, in a 5′- to 3′-order of: SEQ ID NO: 18 and SEQ ID NO: 16, or functional fragments thereof as described herein. In alternative embodiments, the order of the sequence can be, in a 5′- to 3′-order of: SEQ ID NO: 16 and SEQ ID NO: 18, or functional fragments thereof as described herein.

A MDcPxM455 regulatory sequence can have a sequence according to SEQ ID NO: 35. Functional variants thereof may have a sequence that is at least 60%, 65%, 70%, 75%, 80%, 85%, 90%, 95%, 96%, 97%, 98%, or 99% identical thereto.

Functional variants of a MDcPxM455 regulatory cassette as disclosed herein can have a sequence which varies from SEQ ID NO: 35, but which substantially retains activity as a muscle-stem cell specific regulatory cassette. It will be appreciated by the skilled person that it is possible to vary the sequence of a RE while retaining its ability to bind to the requisite transcription factors (TFs) and enhance expression. A functional variant can comprise substitutions, deletions and/or insertions compared to a reference RE, provided they do not render the RE substantially non-functional.

In some embodiments, a functional variant of a MDcPxM455 regulatory cassette as disclosed herein can be viewed as a RC which, when substituted in place of a MDcPxM455, substantially retains its activity. For illustrative purposes only, a MDcPxM455 SCRC which comprises a functional variant of SEQ ID NO: 18 substituted in place of SEQ ID NO: 18 in a MDcPxM455 regulatory cassette as disclosed herein, preferably retains 80% of its activity, more preferably 90% of its activity, more preferably 95% of its activity, and yet more preferably 100% of its activity. Similarly, MDcPxM455 SCRC which comprises a functional variant of SEQ ID NO: 16 substituted in place of SEQ ID NO: 16 in a MDcPxM455 regulatory cassette as disclosed herein, preferably retains 80% of its activity, more preferably 90% of its activity, more preferably 95% of its activity, and yet more preferably 100% of its activity. As an illustrative example only, considering MDcPxM445 MuSC-RC (e.g., see exemplary lower Px/MD MuSC-RC in FIG. 6B), R2-MD in MDcPxM445 can be replaced with a functional variant of R2-MD, and/or the R16-Px7 in MDcPxM445 can be replaced with a functional variant of R2-MD or R16-Px7, respectively, and the MDcPxM445 SCRC substantially retains its activity. Retention of activity can be assessed by comparing expression of a suitable reporter under the control of the reference promoter with an otherwise identical promoter comprising the substituted RE under equivalent conditions.

In some embodiments, the MDcPxM455 RC or a functional variant thereof, has a length of 1050 or fewer nucleotides, e.g., 1000, 900, 800, 700, 600, 500, 400, 300, 200 or 150 or fewer nucleotides.

### Other Px/MD MuSC-RCs

In some embodiments, a Px/MD chimeric MuSC-RC useful in the methods and compositions as disclosed herein comprises R2-MD (SEQ ID NO: 18) and R5-Px7 (SEQ ID NO: 5), or a functional variant thereof that has a sequence that is at least 60%, 65%, 70%, 75%, 80%, 85%, 90%, 95%, 96%, 97%, 98%, or 99% identical to SEQ ID NO: 18 or SEQ ID NO: 5, respectively. In some embodiments, a Px/MD chimeric MuSC-RC useful in the methods and compositions as disclosed herein comprises R2-MD (SEQ ID NO: 18) and R6-Px7 (SEQ ID NO: 6), or a functional variant thereof that has a sequence that is at least 60%, 65%, 70%, 75%, 80%, 85%, 90%, 95%, 96%, 97%, 98%, or 99% identical to SEQ ID NO: 18 or SEQ ID NO: 6, respectively. In some embodiments, a Px/MD chimeric MuSC-RC useful in the methods and compositions as disclosed herein comprises R2-MD (SEQ ID NO: 18) and R4-Px7 (SEQ ID NO: 7), or a functional variant thereof that has a sequence that is at least 60%, 65%, 70%, 75%, 80%, 85%, 90%, 95%, 96%, 97%, 98%, or 99% identical to SEQ ID NO: 18 or SEQ ID NO: 7, respectively.

In some embodiments, a Px/MD chimeric MuSC-RC can comprise R5-MD (SEQ ID NO: 21) or a functional variant thereof that has a sequence that is at least 60%, 65%, 70%, 75%, 80%, 85%, 90%, 95%, 96%, 97%, 98%, or 99% identical to SEQ ID NO: 21, and any of Px7 RE selected from R1-Px7 to R16-Px7.

In some embodiments, a Px/MD chimeric MuSC-RC useful in the methods and compositions as disclosed herein comprises R5-MD (SEQ ID NO: 21) and R5-Px7 (SEQ ID NO: 5), or a functional variant thereof that has a sequence that is at least 60%, 65%, 70%, 75%, 80%, 85%, 90%, 95%, 96%, 97%, 98%, or 99% identical to SEQ ID NO: 21 or SEQ ID NO: 5, respectively. In some embodiments, a Px/MD chimeric MuSC-RC useful in the methods and compositions as disclosed herein comprises R5-MD (SEQ ID NO: 21) and R6-Px7 (SEQ ID NO: 6), or a functional variant thereof that has a sequence that is at least 60%, 65%, 70%, 75%, 80%, 85%, 90%, 95%, 96%, 97%, 98%, or 99% identical to SEQ ID NO: 21 or SEQ ID NO: 6, respectively. In some embodiments, a Px/MD chimeric MuSC-RC useful in the methods and compositions as disclosed herein comprises R5-MD (SEQ ID NO: 21) and R4-Px7 (SEQ ID NO: 7), or a functional variant thereof that has a sequence that is at least 60%, 65%, 70%, 75%, 80%, 85%, 90%, 95%, 96%, 97%, 98%, or 99% identical to SEQ ID NO: 21 or SEQ ID NO: 7, respectively.

In some embodiments, an exemplary Px/MD chimeric MuSC-RC useful in the methods and compositions as disclosed herein comprises R2-MD (SEQ ID NO: 18) and R15-Px7 (SEQ ID NO: 15), or a functional variant thereof that has a sequence that is at least 60%, 65%, 70%, 75%, 80%, 85%, 90%, 95%, 96%, 97%, 98%, or 99% identical to SEQ ID NO: 18 or SEQ ID NO: 15, respectively. It is envisioned that Px/MD chimeric MuSC-RC that comprises R2-MD (SEQ ID NO: 18) and R15-Px7 (SEQ ID NO: 15) or functional variants thereof can occur in any order.

### III. MyoD Regulatory Elements

In reference to FIG. 6A, the MyoD1 gene comprises at least 6 different regulatory elements (RE), referred to as R1-R6 in FIG. 6A, that can be used in a MuSC-RC as disclosed herein. For clarity purposes only, the R1-R6 regulatory elements of the MyoD1 gene illustrated in FIG. 6A are referred to as R1-MD to R6-MD herein.

It will be noted that a functional variant or fragment thereof of any of the R1-MD to R6-MD regulatory elements can be provided on either strand of a double stranded polynucleotide and can be provided in either orientation. As such, complementary and reverse complementary sequences of SEQ ID NO: 17-SEQ ID NO: 22, or a functional variants or fragments thereof fall within the scope of the invention. Single stranded nucleic acids comprising the sequence according to SEQ ID NO: 17-SEQ ID NO: 22 or a functional variant or fragment thereof also fall within the scope of the invention. A functional variant can comprise substitutions, deletions and/or insertions compared to a reference R1-MD to R6-MD regulatory element, provided they do not render the R1-MD to R6-MD regulatory element substantially non-functional. Retention of activity can be assessed by comparing expression of a suitable reporter construct as disclosed herein under the control of the RE or MuSC-RC as disclosed herein with an otherwise identical MuSC-RC comprising the substituted RE under equivalent conditions.

### (a) R1-MD:

R1-MD has a sequence according to SEQ ID NO: 17. Functional variants thereof may have a sequence that is at least 60%, 65%, 70%, 75%, 80%, 85%, 90%, 95%, 96%, 97%, 98%, or 99% identical thereto.

Functional variants of R1-MD are regulatory elements with sequences which vary from R1-MD, but which substantially retain activity as muscle-stem cell specific R1-MD. It will be appreciated by the skilled person that it is possible to vary the sequence of a RE while retaining its ability to bind to the requisite transcription factors (TFs) and enhance expression. A functional variant can comprise substitutions, deletions and/or insertions compared to a reference RE, provided they do not render the RE substantially non-functional.

In some embodiments, a functional variant of R1-MD can be viewed as a RE which, when substituted in place of R1-MD in a MuSC-RC, substantially retains its activity. For example, a SCRC which comprises a functional variant of R1-MD substituted in place of R1-MD preferably retains 80% of its activity, more preferably 90% of its activity, more preferably 95% of its activity, and yet more preferably 100% of its activity. As an illustrative example only, considering MD-1 MuSC-RC (e.g., see exemplary top MD MuSC-RC (also referred to as “MD-689”) in FIG. 6B), R1-MD in MD-1 can be replaced with a functional variant of R1-MD, and the MD-1 SCRC substantially retains its activity. Retention of activity can be assessed by comparing expression of a suitable reporter under the control of the reference promoter with an otherwise identical promoter comprising the substituted RE under equivalent conditions.

In some embodiments, the R1-MD or a functional variant thereof, has a length of 250, or 200 or fewer nucleotides, 150 or fewer nucleotides, 125 or fewer nucleotides, or 100 or fewer nucleotides.

### (b) R2-MD:

R2-MD has a sequence according to SEQ ID NO: 18. Functional variants thereof may have a sequence that is at least 60%, 65%, 70%, 75%, 80%, 85%, 90%, 95%, 96%, 97%, 98%, or 99% identical thereto.

Functional variants of R2-MD are regulatory elements with sequences which vary from R2-MD, but which substantially retain activity as muscle-stem cell specific R2-MD. It will be appreciated by the skilled person that it is possible to vary the sequence of a RE while retaining its ability to bind to the requisite transcription factors (TFs) and enhance expression. A functional variant can comprise substitutions, deletions and/or insertions compared to a reference RE, provided they do not render the RE substantially non-functional.

In some embodiments, a functional variant of R2-MD can be viewed as a RE which, when substituted in place of R2-MD in a MuSC-RC, substantially retains its activity. For example, a SCRC which comprises a functional variant of R2-MD substituted in place of R2-MD preferably retains 80% of its activity, more preferably 90% of its activity, more preferably 95% of its activity, and yet more preferably 100% of its activity. As an illustrative example only, considering MD-1 MuSC-RC (e.g., see exemplary top MD MuSC-RC (also referred to as “MD-689”) in FIG. 6B), R2-MD in MD-1 can be replaced with a functional variant of R2-MD, and the MD-1 SCRC substantially retains its activity. Retention of activity can be assessed by comparing expression of a suitable reporter under the control of the reference promoter with an otherwise identical promoter comprising the substituted RE under equivalent conditions.

It will be noted that the R2-MD or functional variant thereof can be provided on either strand of a double stranded polynucleotide and can be provided in either orientation. As such, complementary and reverse complementary sequences of SEQ ID NO: 18 or a functional variant thereof fall within the scope of the invention. Single stranded nucleic acids comprising the sequence according to SEQ ID NO: 18 or a functional variant thereof also fall within the scope of the invention.

In some embodiments, the R2-MD or a functional variant thereof, has a length of 197 or fewer nucleotides, 150 or fewer nucleotides, 125 or fewer nucleotides, or 100 or fewer nucleotides.

### (c) R3-MD:

R3-MD has a sequence according to SEQ ID NO: 19. Functional variants thereof may have a sequence that is at least 60%, 65%, 70%, 75%, 80%, 85%, 90%, 95%, 96%, 97%, 98%, or 99% identical thereto.

Functional variants of R3-MD are regulatory elements with sequences which vary from R3-MD, but which substantially retain activity as muscle-stem cell specific R3-MD. It will be appreciated by the skilled person that it is possible to vary the sequence of a RE while retaining its ability to bind to the requisite transcription factors (TFs) and enhance expression. A functional variant can comprise substitutions, deletions and/or insertions compared to a reference RE, provided they do not render the RE substantially non-functional.

In some embodiments, a functional variant of R3-MD can be viewed as a RE which, when substituted in place of R3-MD in a MuSC-RC, substantially retains its activity. For example, a SCRC which comprises a functional variant of R3-MD substituted in place of R3-MD preferably retains 80% of its activity, more preferably 90% of its activity, more preferably 95% of its activity, and yet more preferably 100% of its activity. As an illustrative example only, considering MD-1 MuSC-RC (e.g., see exemplary top MD MuSC-RC (also referred to as “MD-689”) in FIG. 6B), R3-MD in MD-1 can be replaced with a functional variant of R1-MD, and the MD-1 SCRC substantially retains its activity. Retention of activity can be assessed by comparing expression of a suitable reporter under the control of the reference promoter with an otherwise identical promoter comprising the substituted RE under equivalent conditions.

It will be noted that the R3-MD or functional variant thereof can be provided on either strand of a double stranded polynucleotide and can be provided in either orientation. As such, complementary and reverse complementary sequences of SEQ ID NO: 19 or a functional variant thereof fall within the scope of the invention. Single stranded nucleic acids comprising the sequence according to SEQ ID NO: 19 or a functional variant thereof also fall within the scope of the invention.

In some embodiments, the R3-MD or a functional variant thereof, has a length of 67 or fewer nucleotides, 60 or fewer nucleotides, 50 or fewer nucleotides, or 40 or fewer nucleotides.

### (d) R4-MD:

R4-MD has a sequence according to SEQ ID NO: 20. Functional variants thereof may have a sequence that is at least 60%, 65%, 70%, 75%, 80%, 85%, 90%, 95%, 96%, 97%, 98%, or 99% identical thereto.

Functional variants of R4-MD are regulatory elements with sequences which vary from R4-MD, but which substantially retain activity as muscle-stem cell specific R4-MD. It will be appreciated by the skilled person that it is possible to vary the sequence of a RE while retaining its ability to bind to the requisite transcription factors (TFs) and enhance expression. A functional variant can comprise substitutions, deletions and/or insertions compared to a reference RE, provided they do not render the RE substantially non-functional.

In some embodiments, a functional variant of R4-MD can be viewed as a RE which, when substituted in place of R4-MD in a MuSC-RC, substantially retains its activity. For example, a SCRC which comprises a functional variant of R3-MD substituted in place of R4-MD preferably retains 80% of its activity, more preferably 90% of its activity, more preferably 95% of its activity, and yet more preferably 100% of its activity. As an illustrative example only, considering MD-1 MuSC-RC (e.g., see exemplary top MD MuSC-RC (also referred to as “MD-689”) in FIG. 6B), R4-MD in MD-1 can be replaced with a functional variant of R1-MD, and the MD-1 SCRC substantially retains its activity. Retention of activity can be assessed by comparing expression of a suitable reporter under the control of the reference promoter with an otherwise identical promoter comprising the substituted RE under equivalent conditions.

It will be noted that the R4-MD or functional variant thereof can be provided on either strand of a double stranded polynucleotide and can be provided in either orientation. As such, complementary and reverse complementary sequences of SEQ ID NO: 20 or a functional variant thereof fall within the scope of the invention. Single stranded nucleic acids comprising the sequence according to SEQ ID NO: 20 or a functional variant thereof also fall within the scope of the invention.

In some embodiments, the R4-MD or a functional variant thereof, has a length of 113 or fewer nucleotides, 100 or fewer nucleotides, 90 or fewer nucleotides, or 80 or fewer nucleotides.

### (e) R5-MD:

R5-MD has a sequence according to SEQ ID NO: 21. Functional variants thereof may have a sequence that is at least 60%, 65%, 70%, 75%, 80%, 85%, 90%, 95%, 96%, 97%, 98%, or 99% identical thereto.

Functional variants of R5-MD are regulatory elements with sequences which vary from R5-MD, but which substantially retain activity as muscle-stem cell specific R5-MD. It will be appreciated by the skilled person that it is possible to vary the sequence of a RE while retaining its ability to bind to the requisite transcription factors (TFs) and enhance expression. A functional variant can comprise substitutions, deletions and/or insertions compared to a reference RE, provided they do not render the RE substantially non-functional.

In some embodiments, a functional variant of R5-MD can be viewed as a RE which, when substituted in place of R5-MD in a MuSC-RC, substantially retains its activity. For example, a SCRC which comprises a functional variant of R5-MD substituted in place of R5-MD preferably retains 80% of its activity, more preferably 90% of its activity, more preferably 95% of its activity, and yet more preferably 100% of its activity. As an illustrative example only, considering MD-1 MuSC-RC (e.g., see exemplary top MD MuSC-RC (also referred to as “MD-689”) in FIG. 6B), R5-MD in MD-1 can be replaced with a functional variant of R1-MD, and the MD-1 SCRC substantially retains its activityRetention of activity can be assessed by comparing expression of a suitable reporter under the control of the reference promoter with an otherwise identical promoter comprising the substituted RE under equivalent conditions.

It will be noted that the R5-MD or functional variant thereof can be provided on either strand of a double stranded polynucleotide and can be provided in either orientation. As such, complementary and reverse complementary sequences of SEQ ID NO: 21 or a functional variant thereof fall within the scope of the invention. Single stranded nucleic acids comprising the sequence according to SEQ ID NO: 21 or a functional variant thereof also fall within the scope of the invention.

In some embodiments, the R5-MD or a functional variant thereof, has a length of 295 or fewer nucleotides, 250 or fewer nucleotides, 200 or fewer nucleotides, or 150 or fewer nucleotides.

### (f) R6-MD:

R6-MD has a sequence according to SEQ ID NO: 22. Functional variants thereof may have a sequence that is at least 60%, 65%, 70%, 75%, 80%, 85%, 90%, 95%, 96%, 97%, 98%, or 99% identical thereto.

Functional variants of R6-MD are regulatory elements with sequences which vary from R6-MD, but which substantially retain activity as muscle-stem cell specific R6-MD. It will be appreciated by the skilled person that it is possible to vary the sequence of a RE while retaining its ability to bind to the requisite transcription factors (TFs) and enhance expression. A functional variant can comprise substitutions, deletions and/or insertions compared to a reference RE, provided they do not render the RE substantially non-functional.

In some embodiments, a functional variant of R6-MD can be viewed as a RE which, when substituted in place of R6-MD in a MuSC-RC, substantially retains its activity. For example, a SCRC which comprises a functional variant of R6-MD substituted in place of R6-MD preferably retains 80% of its activity, more preferably 90% of its activity, more preferably 95% of its activity, and yet more preferably 100% of its activity. As an illustrative example only, considering MD-1 MuSC-RC (e.g., see exemplary top MD MuSC-RC (also referred to as “MD-689”) in FIG. 6B), R6-MD in MD-1 can be replaced with a functional variant of R1-MD, and the MD-1 SCRC substantially retains its activity. Retention of activity can be assessed by comparing expression of a suitable reporter under the control of the reference promoter with an otherwise identical promoter comprising the substituted RE under equivalent conditions.

It will be noted that the R6-MD or functional variant thereof can be provided on either strand of a double stranded polynucleotide and can be provided in either orientation. As such, complementary and reverse complementary sequences of SEQ ID NO: 22 or a functional variant thereof fall within the scope of the invention. Single stranded nucleic acids comprising the sequence according to SEQ ID NO: 22 or a functional variant thereof also fall within the scope of the invention.

In some embodiments, the R6-MD or a functional variant thereof, has a length of 169 or fewer nucleotides, 150 or fewer nucleotides, 125 or fewer nucleotides, or 100 or fewer nucleotides.

### IV. Pax7 Regulatory Elements (Px-RE)

In reference to FIG. 6A, the Pax7 gene comprises at least 16 different regulatory elements (RE), referred to as R1-R16 in FIG. 6A, that can be used in a MuSC-RC as disclosed herein. For clarity purposes only, the R1-R16 regulatory elements of the Pax7 gene as illustrated in FIG. 6A are referred to as R1-Px7 to R16-Px7, respectively herein.

It will be noted that a functional variant or fragment thereof of any of the R1-Px7 to R16-Px7 regulatory elements can be provided on either strand of a double stranded polynucleotide and can be provided in either orientation. As such, complementary and reverse complementary sequences of SEQ ID NO: 1-SEQ ID NO: 16, or a functional variants or fragments thereof fall within the scope of the invention. Single stranded nucleic acids comprising the sequence according to SEQ ID NO: 1-SEQ ID NO: 16 or a functional variant or fragment thereof also fall within the scope of the invention.

Functional variants of SEQ ID NO: 1-SEQ ID NO: 16 are regulatory elements with sequences which vary from SEQ ID NO: 1-SEQ ID NO: 16, respectively, but which substantially retain activity a muscle stem-cell specific RE. It will be appreciated by the skilled person that it is possible to vary the sequence of any of SEQ ID NO: 1-16 while retaining its ability to bind to the requisite transcription factors (TFs) and enhance expression in muscle stem cells as compared to expression in non-muscle cells. A functional variant can comprise substitutions, deletions and/or insertions compared to a reference R1-Px7 to R16-Px7 regulatory element, provided they do not render the R1-Px7 to R16-Px7 regulatory element substantially non-functional. Retention of activity can be assessed by comparing expression of a suitable reporter construct as disclosed herein under the control of the RE or MuSC-RC as disclosed herein with an otherwise identical MuSC-RC comprising the substituted RE under equivalent conditions.

### (a) R1-Px7:

R1-Px7 has a sequence according to SEQ ID NO: 1. Functional variants thereof may have a sequence that is at least 60%, 65%, 70%, 75%, 80%, 85%, 90%, 95%, 96%, 97%, 98%, or 99% identical thereto.

Functional variants of R1-Px7 are regulatory elements with sequences which vary from R1-Px7, but which substantially retain activity as muscle-stem cell specific R1-Px7. It will be appreciated by the skilled person that it is possible to vary the sequence of a RE while retaining its ability to bind to the requisite transcription factors (TFs) and enhance expression. A functional variant can comprise substitutions, deletions and/or insertions compared to a reference RE, provided they do not render the RE substantially non-functional.

In some embodiments, a functional variant of R1-Px7 can be viewed as a RE which, when substituted in place of R1-Px7 in a promoter, substantially retains its activity. For example, a SCRC which comprises a functional variant of R1-Px7 substituted in place of R1-Px7 preferably retains 80% of its activity, more preferably 90% of its activity, more preferably 95% of its activity, and yet more preferably 100% of its activity. As an illustrative example only, considering Px-1 MuSC-RC (e.g., see exemplary top Px MuSC-RC (also referred to as “Px-1861”) in FIG. 6B), R1-Px7 in Px-1 can be replaced with a functional variant of R1-Px7, and the Px-1 MuSC RC substantially retains its activity. Retention of activity can be assessed by comparing expression of a suitable reporter under the control of the reference promoter with an otherwise identical promoter comprising the substituted RE under equivalent conditions.

It will be noted that the R1-Px7 or functional variant thereof can be provided on either strand of a double stranded polynucleotide and can be provided in either orientation. As such, complementary and reverse complementary sequences of SEQ ID NO: 1 or a functional variant thereof fall within the scope of the invention. Single stranded nucleic acids comprising the sequence according to SEQ ID NO: 1 or a functional variant thereof also fall within the scope of the invention.

In some embodiments, the R1-Px7 or a functional variant thereof, has a length of 140 or fewer nucleotides, 120 or fewer nucleotides, 110 or fewer nucleotides, or 100 or fewer nucleotides.

### (b) R2-Px7:

R2-Px7 has a sequence according to SEQ ID NO: 2. Functional variants thereof may have a sequence that is at least 60%, 65%, 70%, 75%, 80%, 85%, 90%, 95%, 96%, 97%, 98%, or 99% identical thereto.

Functional variants of R2-Px7 are regulatory elements with sequences which vary from R2-Px7, but which substantially retain activity as muscle-stem cell specific R2-Px7. It will be appreciated by the skilled person that it is possible to vary the sequence of a RE while retaining its ability to bind to the requisite transcription factors (TFs) and enhance expression. A functional variant can comprise substitutions, deletions and/or insertions compared to a reference RE, provided they do not render the RE substantially non-functional.

In some embodiments, a functional variant of R2-Px7 can be viewed as a RE which, when substituted in place of R2-Px7 in a promoter, substantially retains its activity. For example, a SCRC which comprises a functional variant of R2-Px7 substituted in place of R2-Px7 preferably retains 80% of its activity, more preferably 90% of its activity, more preferably 95% of its activity, and yet more preferably 100% of its activity. As an illustrative example only, considering Px-1 MuSC-RC (e.g., see exemplary top Px MuSC-RC (also referred to as “Px-1861”) in FIG. 6B), R2-Px7 in Px-1 can be replaced with a functional variant of R2-Px7, and the Px-1 MuSC RC (Px-1861) substantially retains its activity. Retention of activity can be assessed by comparing expression of a suitable reporter under the control of the reference promoter with an otherwise identical promoter comprising the substituted RE under equivalent conditions.

It will be noted that the R2-Px7 or functional variant thereof can be provided on either strand of a double stranded polynucleotide and can be provided in either orientation. As such, complementary and reverse complementary sequences of SEQ ID NO: 2 or a functional variant thereof fall within the scope of the invention. Single stranded nucleic acids comprising the sequence according to SEQ ID NO: 2 or a functional variant thereof also fall within the scope of the invention.

In some embodiments, the R2-Px7 or a functional variant thereof, has a length of 140 or fewer nucleotides, 130 or fewer nucleotides, 120 or fewer nucleotides, or 110 or fewer nucleotides.

### (c) R3-Px7:

R3-Px7 has a sequence according to SEQ ID NO: 3. Functional variants thereof may have a sequence that is at least 60%, 65%, 70%, 75%, 80%, 85%, 90%, 95%, 96%, 97%, 98%, or 99% identical thereto.

Functional variants of R3-Px7 are regulatory elements with sequences which vary from R3-Px7, but which substantially retain activity as muscle-stem cell specific R3-Px7. It will be appreciated by the skilled person that it is possible to vary the sequence of a RE while retaining its ability to bind to the requisite transcription factors (TFs) and enhance expression. A functional variant can comprise substitutions, deletions and/or insertions compared to a reference RE, provided they do not render the RE substantially non-functional.

In some embodiments, a functional variant of R3-Px7 can be viewed as a RE which, when substituted in place of R3-Px7 in a promoter, substantially retains its activity. For example, a SCRC which comprises a functional variant of R3-Px7 substituted in place of R3-Px7 preferably retains 80% of its activity, more preferably 90% of its activity, more preferably 95% of its activity, and yet more preferably 100% of its activity. As an illustrative example only, considering Px-1 MuSC-RC (e.g., see exemplary top Px MuSC-RC (also referred to as “Px-1861”) in FIG. 6B), R3-Px7 in Px-1 can be replaced with a functional variant of R3-Px, and the Px-1 MuSC RC substantially retains its activity. Retention of activity can be assessed by comparing expression of a suitable reporter under the control of the reference promoter with an otherwise identical promoter comprising the substituted RE under equivalent conditions.

It will be noted that the R3-Px7 or functional variant thereof can be provided on either strand of a double stranded polynucleotide and can be provided in either orientation. As such, complementary and reverse complementary sequences of SEQ ID NO: 3 or a functional variant thereof fall within the scope of the invention. Single stranded nucleic acids comprising the sequence according to SEQ ID NO: 3 or a functional variant thereof also fall within the scope of the invention.

In some embodiments, the R3-Px7 or a functional variant thereof, has a length of 126 or fewer nucleotides, 110 or fewer nucleotides, 100 or fewer nucleotides, or 90 or fewer nucleotides.

### (d) R4-Px7:

R4-Px7 has a sequence according to SEQ ID NO: 4. Functional variants thereof may have a sequence that is at least 60%, 65%, 70%, 75%, 80%, 85%, 90%, 95%, 96%, 97%, 98%, or 99% identical thereto.

Functional variants of R4-Px7 are regulatory elements with sequences which vary from R4-Px7, but which substantially retain activity as muscle-stem cell specific R4-Px7. It will be appreciated by the skilled person that it is possible to vary the sequence of a RE while retaining its ability to bind to the requisite transcription factors (TFs) and enhance expression. A functional variant can comprise substitutions, deletions and/or insertions compared to a reference RE, provided they do not render the RE substantially non-functional.

In some embodiments, a functional variant of R4-Px7 can be viewed as a RE which, when substituted in place of R4-Px7 in a promoter, substantially retains its activity. For example, a SCRC which comprises a functional variant of R4-Px7 substituted in place of R4-Px7 preferably retains 80% of its activity, more preferably 90% of its activity, more preferably 95% of its activity, and yet more preferably 100% of its activity. As an illustrative example only, considering Px-1 MuSC-RC (e.g., see exemplary top Px MuSC-RC (also referred to as “Px-1861”) in FIG. 6B), R-Px7 in Px-1 can be replaced with a functional variant of R-Px7, and the Px-1 MuSC RC (Px-1861) substantially retains its activity. Retention of activity can be assessed by comparing expression of a suitable reporter under the control of the reference promoter with an otherwise identical promoter comprising the substituted RE under equivalent conditions.

It will be noted that the R4-Px7 or functional variant thereof can be provided on either strand of a double stranded polynucleotide and can be provided in either orientation. As such, complementary and reverse complementary sequences of SEQ ID NO: 4 or a functional variant thereof fall within the scope of the invention. Single stranded nucleic acids comprising the sequence according to SEQ ID NO: 4 or a functional variant thereof also fall within the scope of the invention.

In some embodiments, the R4-Px7 or a functional variant thereof, has a length of 104 or fewer nucleotides, 100 or fewer nucleotides, 90 or fewer nucleotides, or 80 or fewer nucleotides.

### (e) R5-Px7:

R5-Px7 has a sequence according to SEQ ID NO: 5. Functional variants thereof may have a sequence that is at least 60%, 65%, 70%, 75%, 80%, 85%, 90%, 95%, 96%, 97%, 98%, or 99% identical thereto.

Functional variants of R5-Px7 are regulatory elements with sequences which vary from R5-Px7, but which substantially retain activity as muscle-stem cell specific R5-Px7. It will be appreciated by the skilled person that it is possible to vary the sequence of a RE while retaining its ability to bind to the requisite transcription factors (TFs) and enhance expression. A functional variant can comprise substitutions, deletions and/or insertions compared to a reference RE, provided they do not render the RE substantially non-functional.

In some embodiments, a functional variant of R5-Px7 can be viewed as a RE which, when substituted in place of R5-Px7 in a promoter, substantially retains its activity. For example, a SCRC which comprises a functional variant of R5-Px7 substituted in place of R5-Px7 preferably retains 80% of its activity, more preferably 90% of its activity, more preferably 95% of its activity, and yet more preferably 100% of its activity. As an illustrative example only, considering Px-1 MuSC-RC (e.g., see exemplary top Px MuSC-RC (also referred to as “Px-1861”) in FIG. 6B), R5-Px7 in Px-1 can be replaced with a functional variant of R5-Px7, and the Px-1 MuSC RC (Px-1861) substantially retains its activity. Retention of activity can be assessed by comparing expression of a suitable reporter under the control of the reference promoter with an otherwise identical promoter comprising the substituted RE under equivalent conditions.

It will be noted that the R5-Px7 or functional variant thereof can be provided on either strand of a double stranded polynucleotide and can be provided in either orientation. As such, complementary and reverse complementary sequences of SEQ ID NO: 5 or a functional variant thereof fall within the scope of the invention. Single stranded nucleic acids comprising the sequence according to SEQ ID NO: 5 or a functional variant thereof also fall within the scope of the invention.

In some embodiments, the R5-Px7 or a functional variant thereof, has a length of 100 or fewer nucleotides, 90 or fewer nucleotides, 80 or fewer nucleotides, or 700 or fewer nucleotides.

### (f) R6-Px7:

R6-Px7 has a sequence according to SEQ ID NO: 6. Functional variants thereof may have a sequence that is at least 60%, 65%, 70%, 75%, 80%, 85%, 90%, 95%, 96%, 97%, 98%, or 99% identical thereto.

Functional variants of R6-Px7 are regulatory elements with sequences which vary from R6-Px7, but which substantially retain activity as muscle-stem cell specific R6-Px7. It will be appreciated by the skilled person that it is possible to vary the sequence of a RE while retaining its ability to bind to the requisite transcription factors (TFs) and enhance expression. A functional variant can comprise substitutions, deletions and/or insertions compared to a reference RE, provided they do not render the RE substantially non-functional.

In some embodiments, a functional variant of R6-Px7 can be viewed as a RE which, when substituted in place of R6-Px7 in a promoter, substantially retains its activity. For example, a SCRC which comprises a functional variant of R6-Px7 substituted in place of R6-Px7 preferably retains 80% of its activity, more preferably 90% of its activity, more preferably 95% of its activity, and yet more preferably 100% of its activity. As an illustrative example only, considering Px-1 MuSC-RC (e.g., see exemplary top Px MuSC-RC (also referred to as “Px-1861”) in FIG. 6B), R6-Px7 in Px-1 can be replaced with a functional variant of R6-Px7, and the Px-1 MuSC RC (Px-1861) substantially retains its activity. Retention of activity can be assessed by comparing expression of a suitable reporter under the control of the reference promoter with an otherwise identical promoter comprising the substituted RE under equivalent conditions.

It will be noted that the R6-Px7 or functional variant thereof can be provided on either strand of a double stranded polynucleotide and can be provided in either orientation. As such, complementary and reverse complementary sequences of SEQ ID NO: 6 or a functional variant thereof fall within the scope of the invention. Single stranded nucleic acids comprising the sequence according to SEQ ID NO: 6 or a functional variant thereof also fall within the scope of the invention.

In some embodiments, the R6-Px7 or a functional variant thereof, has a length of 372 or fewer nucleotides, 300 or fewer nucleotides, 250 or fewer nucleotides, or 200 or fewer nucleotides.

### (g) R7-Px7:

R7-Px7 has a sequence according to SEQ ID NO: 7. Functional variants thereof may have a sequence that is at least 60%, 65%, 70%, 75%, 80%, 85%, 90%, 95%, 96%, 97%, 98%, or 99% identical thereto.

Functional variants of R7-Px7 are regulatory elements with sequences which vary from R2-Px7, but which substantially retain activity as muscle-stem cell specific R2-Px7. It will be appreciated by the skilled person that it is possible to vary the sequence of a RE while retaining its ability to bind to the requisite transcription factors (TFs) and enhance expression. A functional variant can comprise substitutions, deletions and/or insertions compared to a reference RE, provided they do not render the RE substantially non-functional.

In some embodiments, a functional variant of R7-Px7 can be viewed as a RE which, when substituted in place of R7-Px7 in a promoter, substantially retains its activity. For example, a SCRC which comprises a functional variant of R7-Px7 substituted in place of R7-Px7 preferably retains 80% of its activity, more preferably 90% of its activity, more preferably 95% of its activity, and yet more preferably 100% of its activity. As an illustrative example only, considering Px-1 MuSC-RC (e.g., see exemplary top Px MuSC-RC (also referred to as “Px-1861”) in FIG. 6B), R7-Px7 in Px-1 can be replaced with a functional variant of R7-Px7, and the Px-1 MuSC RC (Px-1861) substantially retains its activity. Retention of activity can be assessed by comparing expression of a suitable reporter under the control of the reference promoter with an otherwise identical promoter comprising the substituted RE under equivalent conditions.

It will be noted that the R7-Px7 or functional variant thereof can be provided on either strand of a double stranded polynucleotide and can be provided in either orientation. As such, complementary and reverse complementary sequences of SEQ ID NO: 7 or a functional variant thereof fall within the scope of the invention. Single stranded nucleic acids comprising the sequence according to SEQ ID NO: 7 or a functional variant thereof also fall within the scope of the invention.

In some embodiments, the R7-Px7 or a functional variant thereof, has a length of 164 or fewer nucleotides, 150 or fewer nucleotides, 125 or fewer nucleotides, or 100 or fewer nucleotides.

### (h) R8-Px7:

R8-Px7 has a sequence according to SEQ ID NO: 8. Functional variants thereof may have a sequence that is at least 60%, 65%, 70%, 75%, 80%, 85%, 90%, 95%, 96%, 97%, 98%, or 99% identical thereto.

Functional variants of R8-Px7 are regulatory elements with sequences which vary from R2-Px7, but which substantially retain activity as muscle-stem cell specific R2-Px7. It will be appreciated by the skilled person that it is possible to vary the sequence of a RE while retaining its ability to bind to the requisite transcription factors (TFs) and enhance expression. A functional variant can comprise substitutions, deletions and/or insertions compared to a reference RE, provided they do not render the RE substantially non-functional.

In some embodiments, a functional variant of R8-Px7 can be viewed as a RE which, when substituted in place of R8-Px7 in a promoter, substantially retains its activity. For example, a SCRC which comprises a functional variant of R8-Px7 substituted in place of R8-Px7 preferably retains 80% of its activity, more preferably 90% of its activity, more preferably 95% of its activity, and yet more preferably 100% of its activity. As an illustrative example only, considering Px-1 MuSC-RC (e.g., see exemplary top Px MuSC-RC (also referred to as “Px-1861”) in FIG. 6B), R8-Px7 in Px-1 can be replaced with a functional variant of R8-Px7, and the Px-1 MuSC RC (Px-1861) substantially retains its activity. Retention of activity can be assessed by comparing expression of a suitable reporter under the control of the reference promoter with an otherwise identical promoter comprising the substituted RE under equivalent conditions.

It will be noted that the R8-Px7 or functional variant thereof can be provided on either strand of a double stranded polynucleotide and can be provided in either orientation. As such, complementary and reverse complementary sequences of SEQ ID NO: 8 or a functional variant thereof fall within the scope of the invention. Single stranded nucleic acids comprising the sequence according to SEQ ID NO: 8 or a functional variant thereof also fall within the scope of the invention.

In some embodiments, the R8-Px7 or a functional variant thereof, has a length of 66 or fewer nucleotides, 50 or fewer nucleotides, 40 or fewer nucleotides, or 30 or fewer nucleotides.

### (i) R9-Px7:

R9-Px7 has a sequence according to SEQ ID NO: 9. Functional variants thereof may have a sequence that is at least 60%, 65%, 70%, 75%, 80%, 85%, 90%, 95%, 96%, 97%, 98%, or 99% identical thereto.

Functional variants of R9-Px7 are regulatory elements with sequences which vary from R9-Px7, but which substantially retain activity as muscle-stem cell specific R9-Px7. It will be appreciated by the skilled person that it is possible to vary the sequence of a RE while retaining its ability to bind to the requisite transcription factors (TFs) and enhance expression. A functional variant can comprise substitutions, deletions and/or insertions compared to a reference RE, provided they do not render the RE substantially non-functional.

In some embodiments, a functional variant of R9-Px7 can be viewed as a RE which, when substituted in place of R9-Px7 in a promoter, substantially retains its activity. For example, a SCRC which comprises a functional variant of R9-Px7 substituted in place of R9-Px7 preferably retains 80% of its activity, more preferably 90% of its activity, more preferably 95% of its activity, and yet more preferably 100% of its activity. As an illustrative example only, considering Px-1 MuSC-RC (e.g., see exemplary top Px MuSC-RC (also referred to as “Px-1861”) in FIG. 6B), R9-Px7 in Px-1 can be replaced with a functional variant of R9-Px7, and the Px-1 MuSC RC (Px-1861) substantially retains its activity. Retention of activity can be assessed by comparing expression of a suitable reporter under the control of the reference promoter with an otherwise identical promoter comprising the substituted RE under equivalent conditions.

It will be noted that the R9-Px7 or functional variant thereof can be provided on either strand of a double stranded polynucleotide and can be provided in either orientation. As such, complementary and reverse complementary sequences of SEQ ID NO: 9 or a functional variant thereof fall within the scope of the invention. Single stranded nucleic acids comprising the sequence according to SEQ ID NO: 9 or a functional variant thereof also fall within the scope of the invention.

In some embodiments, the R9-Px7 or a functional variant thereof, has a length of 81 or fewer nucleotides, 70 or fewer nucleotides, 60 or fewer nucleotides, or 50 or fewer nucleotides.

### (j) R10-Px7:

R10-Px7 has a sequence according to SEQ ID NO: 10. Functional variants thereof may have a sequence that is at least 60%, 65%, 70%, 75%, 80%, 85%, 90%, 95%, 96%, 97%, 98%, or 99% identical thereto.

Functional variants of R10-Px7 are regulatory elements with sequences which vary from R10-Px7, but which substantially retain activity as muscle-stem cell specific R10-Px7. It will be appreciated by the skilled person that it is possible to vary the sequence of a RE while retaining its ability to bind to the requisite transcription factors (TFs) and enhance expression. A functional variant can comprise substitutions, deletions and/or insertions compared to a reference RE, provided they do not render the RE substantially non-functional.

In some embodiments, a functional variant of R10-Px7 can be viewed as a RE which, when substituted in place of R10-Px7 in a promoter, substantially retains its activity. For example, a SCRC which comprises a functional variant of R10-Px7 substituted in place of R10-Px7 preferably retains 80% of its activity, more preferably 90% of its activity, more preferably 95% of its activity, and yet more preferably 100% of its activity. As an illustrative example only, considering Px-1 MuSC-RC (e.g., see exemplary top Px MuSC-RC (also referred to as “Px-1861”) in FIG. 6B), R10-Px7 in Px-1 can be replaced with a functional variant of R10-Px7, and the Px-1 MuSC RC (Px-1861) substantially retains its activity. Retention of activity can be assessed by comparing expression of a suitable reporter under the control of the reference promoter with an otherwise identical promoter comprising the substituted RE under equivalent conditions.

It will be noted that the R10-Px7 or functional variant thereof can be provided on either strand of a double stranded polynucleotide and can be provided in either orientation. As such, complementary and reverse complementary sequences of SEQ ID NO: 10 or a functional variant thereof fall within the scope of the invention. Single stranded nucleic acids comprising the sequence according to SEQ ID NO: 10 or a functional variant thereof also fall within the scope of the invention.

In some embodiments, the R10-Px7 or a functional variant thereof, has a length of 79 or fewer nucleotides, 70 or fewer nucleotides, 60 or fewer nucleotides, or 50 or fewer nucleotides.

### (k) R11-Px7:

R11-Px7 has a sequence according to SEQ ID NO: 11. Functional variants thereof may have a sequence that is at least 60%, 65%, 70%, 75%, 80%, 85%, 90%, 95%, 96%, 97%, 98%, or 99% identical thereto.

Functional variants of R 1-Px7 are regulatory elements with sequences which vary from R 11-Px7, but which substantially retain activity as muscle-stem cell specific R11-Px7. It will be appreciated by the skilled person that it is possible to vary the sequence of a RE while retaining its ability to bind to the requisite transcription factors (TFs) and enhance expression. A functional variant can comprise substitutions, deletions and/or insertions compared to a reference RE, provided they do not render the RE substantially non-functional.

In some embodiments, a functional variant of R11-Px7 can be viewed as a RE which, when substituted in place of R11-Px7 in a promoter, substantially retains its activity. For example, a SCRC which comprises a functional variant of R11-Px7 substituted in place of R11-Px7 preferably retains 80% of its activity, more preferably 90% of its activity, more preferably 95% of its activity, and yet more preferably 100% of its activity. As an illustrative example only, considering Px-1 MuSC-RC (e.g., see exemplary top Px MuSC-RC (also referred to as “Px-1861”) in FIG. 6B), R 11-Px7 in Px-1 can be replaced with a functional variant of R11-Px7, and the Px-1 MuSC RC (Px-1861) substantially retains its activity. Retention of activity can be assessed by comparing expression of a suitable reporter under the control of the reference promoter with an otherwise identical promoter comprising the substituted RE under equivalent conditions.

It will be noted that the R11-Px7 or functional variant thereof can be provided on either strand of a double stranded polynucleotide and can be provided in either orientation. As such, complementary and reverse complementary sequences of SEQ ID NO: 11 or a functional variant thereof fall within the scope of the invention. Single stranded nucleic acids comprising the sequence according to SEQ ID NO: 11 or a functional variant thereof also fall within the scope of the invention.

In some embodiments, the R11-Px7 or a functional variant thereof, has a length of 103 or fewer nucleotides, 90 or fewer nucleotides, 80 or fewer nucleotides, or 70 or fewer nucleotides.

### (l) R12-Px7:

R12-Px7 has a sequence according to SEQ ID NO: 12. Functional variants thereof may have a sequence that is at least 60%, 65%, 70%, 75%, 80%, 85%, 90%, 95%, 96%, 97%, 98%, or 99% identical thereto.

Functional variants of R12-Px7 are regulatory elements with sequences which vary from R12-Px7, but which substantially retain activity as muscle-stem cell specific R12-Px7. It will be appreciated by the skilled person that it is possible to vary the sequence of a RE while retaining its ability to bind to the requisite transcription factors (TFs) and enhance expression. A functional variant can comprise substitutions, deletions and/or insertions compared to a reference RE, provided they do not render the RE substantially non-functional.

In some embodiments, a functional variant of R12-Px7 can be viewed as a RE which, when substituted in place of R12-Px7 in a promoter, substantially retains its activity. For example, a SCRC which comprises a functional variant of R12-Px7 substituted in place of R12-Px7 preferably retains 80% of its activity, more preferably 90% of its activity, more preferably 95% of its activity, and yet more preferably 100% of its activity As an illustrative example only, considering Px-1 MuSC-RC (e.g., see exemplary top Px MuSC-RC (also referred to as “Px-1861”) in FIG. 6B), R12-Px7 in Px-1 can be replaced with a functional variant of R12-Px7, and the Px-1 MuSC RC (Px-1861) substantially retains its activity. Retention of activity can be assessed by comparing expression of a suitable reporter under the control of the reference promoter with an otherwise identical promoter comprising the substituted RE under equivalent conditions.

It will be noted that the R12-Px7 or functional variant thereof can be provided on either strand of a double stranded polynucleotide and can be provided in either orientation. As such, complementary and reverse complementary sequences of SEQ ID NO: 12 or a functional variant thereof fall within the scope of the invention. Single stranded nucleic acids comprising the sequence according to SEQ ID NO: 12 or a functional variant thereof also fall within the scope of the invention.

In some embodiments, the R12-Px7 or a functional variant thereof, has a length of 57 or fewer nucleotides, 50 or fewer nucleotides, 40 or fewer nucleotides, or 30 or fewer nucleotides.

### (m) R13-Px7:

R13-Px7 has a sequence according to SEQ ID NO: 13. Functional variants thereof may have a sequence that is at least 60%, 65%, 70%, 75%, 80%, 85%, 90%, 95%, 96%, 97%, 98%, or 99% identical thereto.

Functional variants of R13-Px7 are regulatory elements with sequences which vary from R13-Px7, but which substantially retain activity as muscle-stem cell specific R13-Px7. It will be appreciated by the skilled person that it is possible to vary the sequence of a RE while retaining its ability to bind to the requisite transcription factors (TFs) and enhance expression. A functional variant can comprise substitutions, deletions and/or insertions compared to a reference RE, provided they do not render the RE substantially non-functional.

In some embodiments, a functional variant of R13-Px7 can be viewed as a RE which, when substituted in place of R13-Px7 in a promoter, substantially retains its activity. For example, a SCRC which comprises a functional variant of R13-Px7 substituted in place of R13-Px7 preferably retains 80% of its activity, more preferably 90% of its activity, more preferably 95% of its activity, and yet more preferably 100% of its activity. As an illustrative example only, considering Px-1 MuSC-RC (e.g., see exemplary top Px MuSC-RC (also referred to as “Px-1861”) in FIG. 6B), R13-Px7 in Px-1 can be replaced with a functional variant of R13-Px7, and the Px-1 MuSC RC (Px-1861) substantially retains its activity. Retention of activity can be assessed by comparing expression of a suitable reporter under the control of the reference promoter with an otherwise identical promoter comprising the substituted RE under equivalent conditions.

It will be noted that the R13-Px7 or functional variant thereof can be provided on either strand of a double stranded polynucleotide and can be provided in either orientation. As such, complementary and reverse complementary sequences of SEQ ID NO: 13 or a functional variant thereof fall within the scope of the invention. Single stranded nucleic acids comprising the sequence according to SEQ ID NO: 13 or a functional variant thereof also fall within the scope of the invention.

In some embodiments, the R13-Px7 or a functional variant thereof, has a length of 115 or fewer nucleotides, 100 or fewer nucleotides, 90 or fewer nucleotides, or 75 or fewer nucleotides.

### (n) R14-Px7:

R14-Px7 has a sequence according to SEQ ID NO: 14. Functional variants thereof may have a sequence that is at least 60%, 65%, 70%, 75%, 80%, 85%, 90%, 95%, 96%, 97%, 98%, or 99% identical thereto.

Functional variants of R14-Px7 are regulatory elements with sequences which vary from R14-Px7, but which substantially retain activity as muscle-stem cell specific R14-Px7. It will be appreciated by the skilled person that it is possible to vary the sequence of a RE while retaining its ability to bind to the requisite transcription factors (TFs) and enhance expression. A functional variant can comprise substitutions, deletions and/or insertions compared to a reference RE, provided they do not render the RE substantially non-functional.

In some embodiments, a functional variant of R14-Px7 can be viewed as a RE which, when substituted in place of R14-Px7 in a promoter, substantially retains its activity. For example, a SCRC which comprises a functional variant of R14-Px7 substituted in place of R14-Px7 preferably retains 80% of its activity, more preferably 90% of its activity, more preferably 95% of its activity, and yet more preferably 100% of its activity. As an illustrative example only, considering Px-1 MuSC-RC (e.g., see exemplary top Px MuSC-RC (also referred to as “Px-1861”) in FIG. 6B), R14-Px7 in Px-1 can be replaced with a functional variant of R14-Px7, and the Px-1 MuSC RC (Px-1861) substantially retains its activity. Retention of activity can be assessed by comparing expression of a suitable reporter under the control of the reference promoter with an otherwise identical promoter comprising the substituted RE under equivalent conditions.

It will be noted that the R14-Px7 or functional variant thereof can be provided on either strand of a double stranded polynucleotide and can be provided in either orientation. As such, complementary and reverse complementary sequences of SEQ ID NO: 14 or a functional variant thereof fall within the scope of the invention. Single stranded nucleic acids comprising the sequence according to SEQ ID NO: 14 or a functional variant thereof also fall within the scope of the invention.

In some embodiments, the R14-Px7 or a functional variant thereof, has a length of 139 or fewer nucleotides, 120 or fewer nucleotides, 110 or fewer nucleotides, or 100 or fewer nucleotides.

### (o) R15-Px7:

R15-Px7 has a sequence according to SEQ ID NO: 15. Functional variants thereof may have a sequence that is at least 60%, 65%, 70%, 75%, 80%, 85%, 90%, 95%, 96%, 97%, 98%, or 99% identical thereto.

Functional variants of R15-Px7 are regulatory elements with sequences which vary from R15-Px7, but which substantially retain activity as muscle-stem cell specific R15-Px7. It will be appreciated by the skilled person that it is possible to vary the sequence of a RE while retaining its ability to bind to the requisite transcription factors (TFs) and enhance expression. A functional variant can comprise substitutions, deletions and/or insertions compared to a reference RE, provided they do not render the RE substantially non-functional.

In some embodiments, a functional variant of R16-Px7 can be viewed as a RE which, when substituted in place of R15-Px7 in a promoter, substantially retains its activity. For example, a SCRC which comprises a functional variant of R15-Px7 substituted in place of R15-Px7 preferably retains 80% of its activity, more preferably 90% of its activity, more preferably 95% of its activity, and yet more preferably 100% of its activity. As an illustrative example only, considering Px-1 MuSC-RC (e.g., see exemplary top Px MuSC-RC (also referred to as “Px-1861”) in FIG. 6B), R15-Px7 in Px-1 can be replaced with a functional variant of R15-Px7, and the Px-1 MuSC RC (Px-1861) substantially retains its activity. Retention of activity can be assessed by comparing expression of a suitable reporter under the control of the reference promoter with an otherwise identical promoter comprising the substituted RE under equivalent conditions.

It will be noted that the R15-Px7 or functional variant thereof can be provided on either strand of a double stranded polynucleotide and can be provided in either orientation. As such, complementary and reverse complementary sequences of SEQ ID NO: 15 or a functional variant thereof fall within the scope of the invention. Single stranded nucleic acids comprising the sequence according to SEQ ID NO: 15 or a functional variant thereof also fall within the scope of the invention.

In some embodiments, the R15-Px7 or a functional variant thereof, has a length of 500 or fewer nucleotides, 400 or fewer nucleotides, 300 or fewer nucleotides, or 200 or fewer nucleotides.

### (p) R16-Px7:

R16-Px7 has a sequence according to SEQ ID NO: 16. Functional variants thereof may have a sequence that is at least 60%, 65%, 70%, 75%, 80%, 85%, 90%, 95%, 96%, 97%, 98%, or 99% identical thereto.

Functional variants of R16-Px7 are regulatory elements with sequences which vary from R16-Px7, but which substantially retain activity as muscle-stem cell specific R16-Px7. It will be appreciated by the skilled person that it is possible to vary the sequence of a RE while retaining its ability to bind to the requisite transcription factors (TFs) and enhance expression. A functional variant can comprise substitutions, deletions and/or insertions compared to a reference RE, provided they do not render the RE substantially non-functional.

In some embodiments, a functional variant of R16-Px7 can be viewed as a RE which, when substituted in place of R16-Px7 in a promoter, substantially retains its activity. For example, a SCRC which comprises a functional variant of R16-Px7 substituted in place of R16-Px7 preferably retains 80% of its activity, more preferably 90% of its activity, more preferably 95% of its activity, and yet more preferably 100% of its activity. As an illustrative example only, considering Px-1 MuSC-RC (e.g., see exemplary top Px MuSC-RC (also referred to as “Px-1861”) in FIG. 6B), R16-Px7 in Px-1 can be replaced with a functional variant of R16-Px7, and the Px-1 MuSC RC (Px-1861) substantially retains its activity. Retention of activity can be assessed by comparing expression of a suitable reporter under the control of the reference promoter with an otherwise identical promoter comprising the substituted RE under equivalent conditions.

It will be noted that the R16-Px7 or functional variant thereof can be provided on either strand of a double stranded polynucleotide and can be provided in either orientation. As such, complementary and reverse complementary sequences of SEQ ID NO: 16 or a functional variant thereof fall within the scope of the invention. Single stranded nucleic acids comprising the sequence according to SEQ ID NO: 16 or a functional variant thereof also fall within the scope of the invention.

In some embodiments, the R16-Px7 or a functional variant thereof, has a length of 847 or fewer nucleotides, 750 or fewer nucleotides, 600 or fewer nucleotides, or 500 or fewer nucleotides.

### V. Assays to Determine the Expression in MuSC

In some embodiments, MuSC-RC have at least 2-fold, or at least 3-fold, at least 10-fold, at least 30-fold, at least 100-fold, at least 300-fold, at least 1000-fold more transcriptional activity in muscle stem cells than differentiated myocytes.

In some embodiments, a nucleic acid operatively linked to a MuSC-RC as disclosed herein has higher expression in skeletal muscle cells as compared to a nucleic acid sequence operatively linked to a CK8e promoter (SEQ ID NO: 306).

The MuSC-RC as disclosed herein are selected based on expression in the targeted cell, e.g., muscle stem cells (MuSC), preferably skeletal muscle stem cell. When a gene is selectively expressed in targeted muscle stem cells and is not substantially expressed in non-targeted cells (e.g., differentiated muscle cells), the product of the coding sequence is preferentially expressed in the targeted cell type. In particular embodiments, selective expression is greater than 50% expression in muscle stem cells as compared to a reference cell type (e.g., non-muscle cells); greater than 60% expression as compared to a reference cell type; greater than 70% expression as compared to a reference cell type; greater than 80% expression as compared to a reference cell type; or greater than 90% expression as compared to a reference cell type. In particular embodiments, a reference cell type refers to non-stem muscle cells. In particular embodiments, a reference cell type is a non-stem muscle cell, and can be within an anatomical structure that is the same, or adjacent to, an anatomical structure that includes the targeted muscle stem cell type.

In some embodiments, the product of the coding sequence may be expressed at low levels in non-selected and/or reference cell types, for example at less than 1% or 1%, 2%, 3%, 5%, 10%, 15% or 20% of the levels at which the product is expressed in targeted cells. In particular embodiments, the targeted muscle cell type is the only cell type that expresses the right combination of transcription factors that bind to the MuSC-RC as disclosed herein to drive gene expression. Thus, in particular embodiments, expression occurs exclusively within the targeted muscle stem cell types. In some embodiments, expression does not occur in non-stem muscle cells.

Different Muscle-specific expression constructs (MSEC), their makeup, and how their activity is measured are described in, e.g., PCT/US2022/023915, which is incorporated herein by reference in its entirety. Similar approaches can be applied by one of ordinary skill in the art to measure the MuSC activity of a MuSC-RC disclosed herein, using muscle stem cells as a host for reporter constructs.

It will be appreciated that the ability of a given MuSC-RE to function as a muscle stem cell-specific promoter is determined significantly by the ability of the sequence to bind the some of the TFs disclosed herein in Table 3, that bind to the MuSC-RE nucleic acid sequence. Accordingly, in most cases, assessing the ability of a MuSC-RE or a functional variant or fragment of a MuSC-RE to function as a muscle stem cell-specific promoter assess if they contain one or more transcription factor binding sites (TFBS) for the most or all of same TFs as the reference MuSC-RE sequence. It is preferred, but not essential, that the TFBS of a functional variant of a MuSC-RE are in the same relative positions (i.e. order and general position) as the reference MuSC-RE. It is also preferred, but not essential, that the TFBS of a functional variant are in the same orientation as the reference sequence MuSC-RE (it will be noted that TFBS can in some cases be present in reverse orientation, e.g. as the reverse complement vis-á-vis the sequence in the reference sequence). It is also preferred, but not essential, that the TFBS of a functional variant of a MuSC-RE are on the same strand as the reference sequence. Thus, in preferred embodiments, the functional variant of a MuSC-RE comprises TFBS for the same TFs, in the same order, the same position, in the same orientation and on the same strand as the reference sequence. It will also be appreciated that the sequences lying between TFBS (referred to in some cases as spacer sequences, or suchlike) are of less consequence to the function of the MuSC-RE. Such sequences can typically be varied considerably, and their lengths can be altered. However, in one embodiments the spacing (i.e. the distance between adjacent TFBS) is substantially the same (e.g. it does not vary by more than 20%, preferably by not more than 10%, and more preferably it is approximately the same) in a functional variant of a MuSC-RE as it is in the reference MuSC-RE sequence. It will be apparent that in some cases a functional variant of a MuSC-RE can be present in the reverse orientation, e.g. it can be the reverse complement of a CRE as described above, or a variant thereof.

Levels of sequence identity between a functional variant of a MuSC-RE and the reference MuSC-RE sequence can also be an indicator or retained functionality. High levels of sequence identity in the TFBS of MuSC-RE is of generally higher importance than sequence identity in the spacer sequences (where there is little or no requirement for any conservation of sequence). However, it will be appreciated that even within the TFBS, a considerable degree of sequence variation can be accommodated, given that the sequence of a functional TFBS does not need to exactly match the consensus sequence.

The ability of one or more TFs to bind to a TFBS in a given functional variant can determined by any relevant means known in the art, including, but not limited to, electromobility shift assays (EMSA), binding assays, chromatin immunoprecipitation (ChIP), and ChIP-sequencing (ChIP-seq). In a preferred embodiment the ability of one or more TFs to bind a given functional variant is determined by EMSA.

Methods of performing EMSA are well-known in the art. Suitable approaches are described in Sambrook et al. cited above. Many relevant articles describing this procedure are available, e.g. Hellman and Fried, Nat Protoc. 2007; 2(8): 1849-1861.

As disclosed herein, “Muscle stem cell-specific” or “Muscle stem cell-specific expression” refers to the ability of a MuSC-RE to enhance or drive expression of a gene in the muscle stem cell or satellite cell (or in a muscle-derived cells) in a preferential or predominant manner as compared to other non-muscle tissues (e.g. fibroblasts, spleen, liver, lung, and brain). Expression of the gene can be in the form of mRNA or protein. In preferred embodiments, muscle stem cell-specific expression is such that there is negligible expression in other (i.e. non-muscle) tissues or cells, i.e. expression is highly muscle-specific, and notably preferentially highly specific to muscle stem cells.

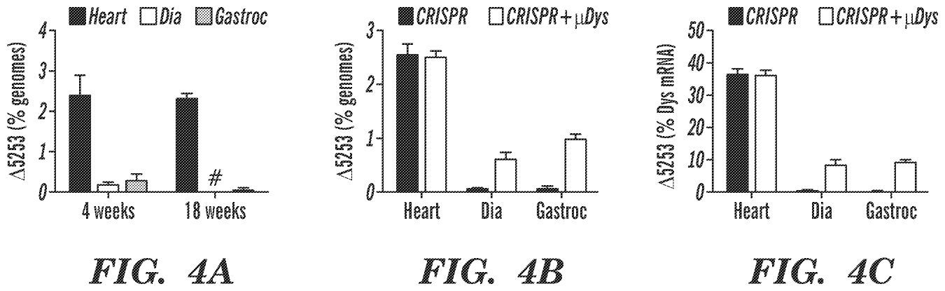

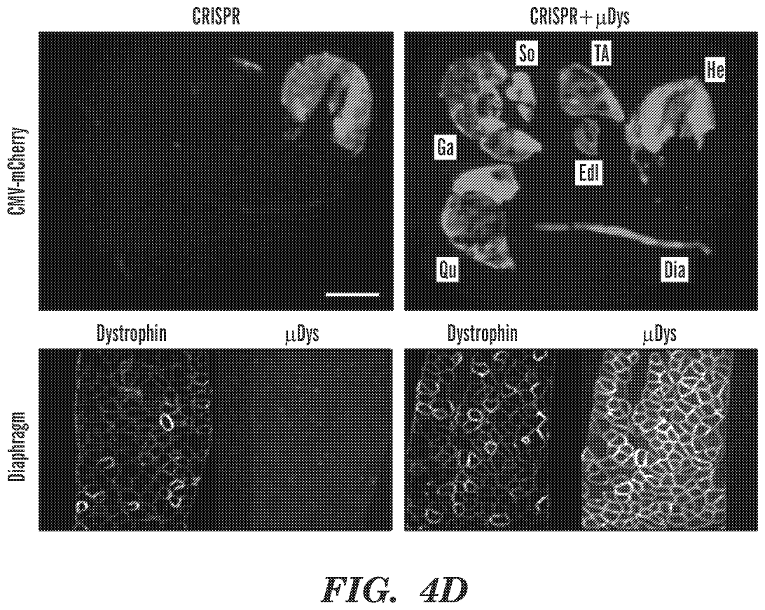

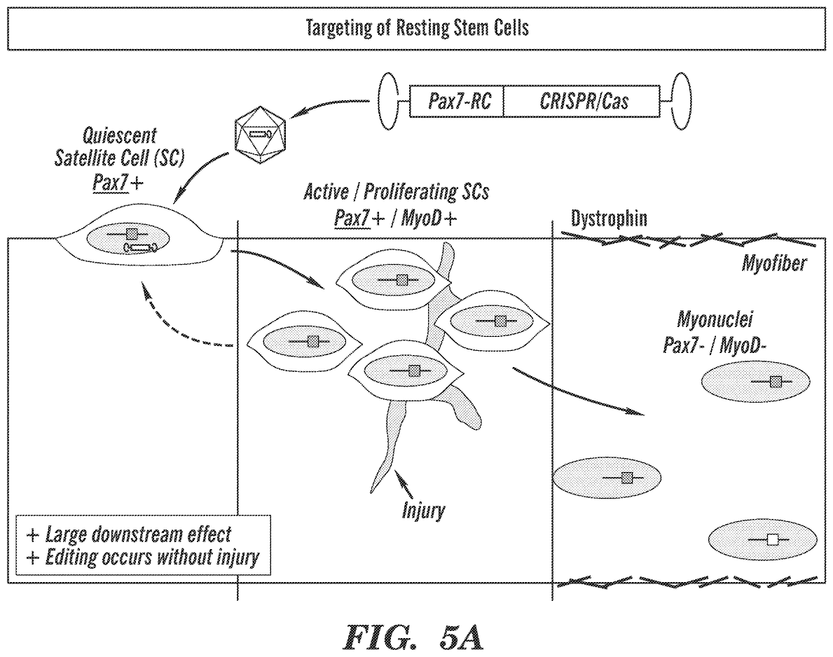

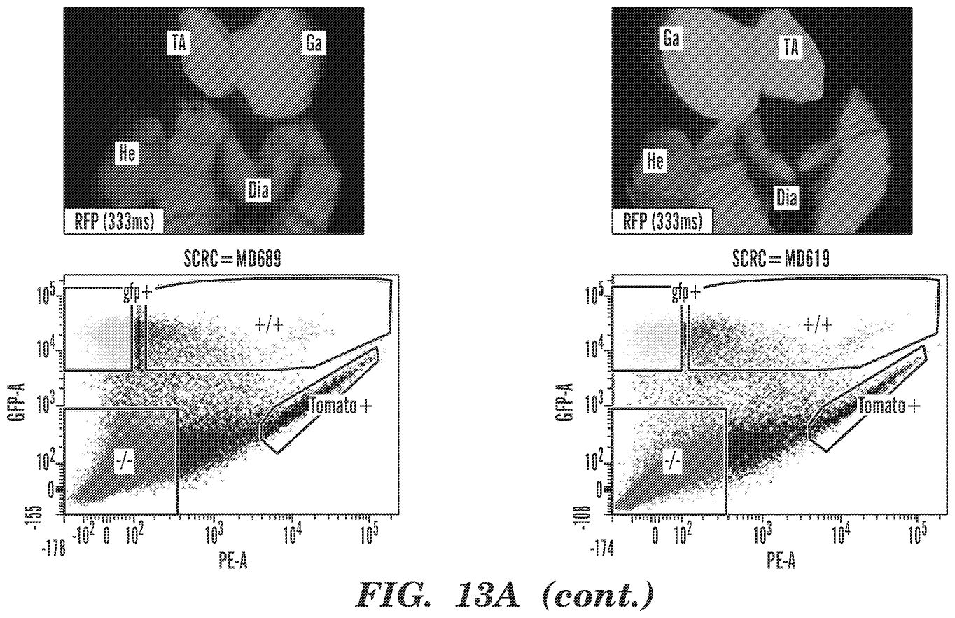

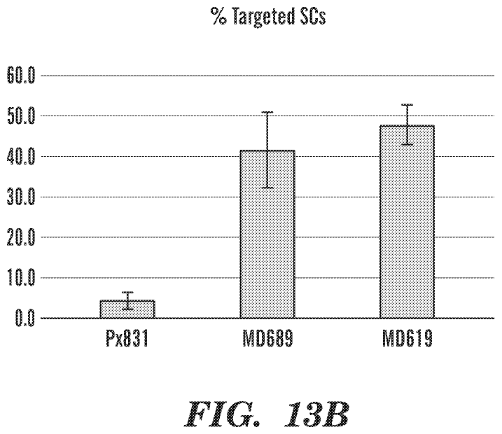

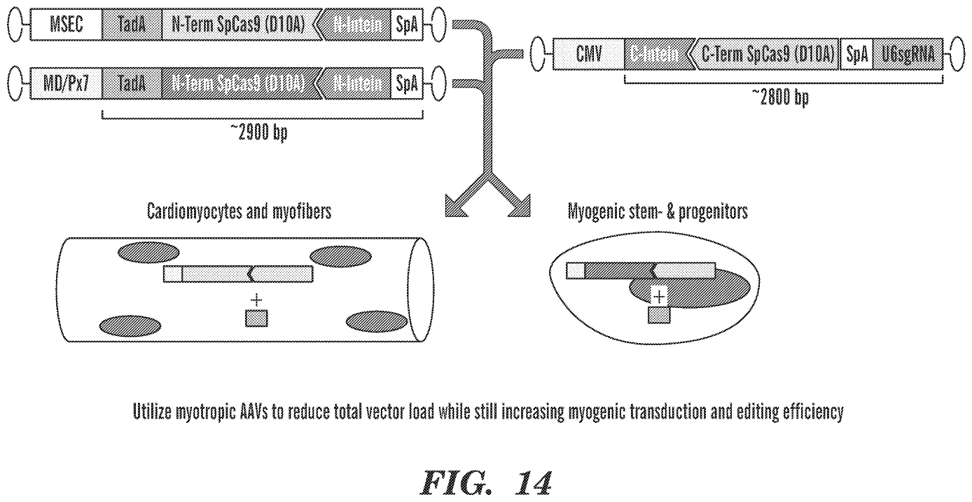

The term “muscle stem cell-specific” or “satellite-specific expression” refers to the ability of a MuSC-RE to enhance or drive expression of a gene in muscle stem cells in a preferential or predominant manner as compared to other tissues (e.g. spleen, liver, lung, and brain) and compared to mature skeletal muscle tissue. Muscle stem cell-specificity can be identified wherein the expression of a gene (e.g. a therapeutic or reporter gene) occurs preferentially or predominantly in muscle stem cells. Preferential or predominant expression can be defined, for example, where the level of expression is significantly greater in muscle stem cells than in other types of cells (i.e. non-stem cells, or non-muscle cells). For example, expression in muscle stem cells is suitably at least 5-fold higher than in non-stem cells, preferably at least 10-fold higher than in non-stem cells, and it may be 50-fold higher or more in some cases. For convenience, muscle stem cell-specific expression can suitably be demonstrated via a comparison of expression levels in different tissues, e.g., see FIGS. 4D and 13A-13B, as well as primary myocytes or SC isolated from mice administered a vector (e.g., AAV vector comprising the MuSC-RE operatively linked to a reporter transgene).

The synthetic muscle stem cell-specific MuSC-RCs as disclosed herein exhibit reduced expression in non-muscle-derived cells, suitably in Huh7, HEK-293, HeLa, and/or A549 cells when compared to a non-tissue specific promoter such as CMV (SEQ ID NO: 64). The synthetic MuSC-RCs preferably have an activity of 50% or less than the CMV promoter in non-muscle-derived cells, suitably 25% or less, 20% or less, 15% or less, 10% or less, 5% or less or 1% or less. Generally, it is preferred that expression in non-muscle-derived cells is minimized, but in some cases this may not be necessary. Even if a synthetic MuSC-RC as disclosed herein has higher expression in, e.g., one or two non-muscle cells, as long as it generally has higher expression overall in a range of muscle stem cells versus non-muscle cell, it can still a muscle stem cell-specific promoter. In some embodiments, a MuSC-RCs expresses a gene at least 25%, or at least 35%, or at least 45%, or at least 55%, or at least 65%, or at least 75%, or at least 80%, or at least 85%, or at least 90%, or at least 95%, or any integer between 25%-95% higher in muscle stem cells as compared to non-muscle cells.

The synthetic MuSC-RCs as disclosed herein are preferably suitable for promoting expression in muscle stem cells of a subject, e.g. driving muscle stem cell-specific expression of a transgene, preferably a therapeutic transgene. The synthetic MuSC-RCs as disclosed herein are suitable for promoting expression in satellite cells in skeletal muscles of a subject, e.g. driving muscle stem cell-specific expression of a transgene, preferably a therapeutic transgene, in activated quiescent SC and/or activated or early activated muscle satellite cells as described herein. Preferred synthetic muscle stem cell-specific MuSC-RCs as disclosed herein are suitable for promoting muscle stem cell-specific transgene expression and have an activity in muscle satellite cells which is at least 15%, 20%, 30%, 40%, 50%, 60%, 70%, 80%, 90%, 100%, 125%, 150%, 175%, 200%, 250%, 300%, 350% or 400% greater than the activity of the CK8e promoter (SEQ ID NO: 306).

Synthetic MuSC-RCs as disclosed herein may also be able to promote muscle stem cell-specific expression of a gene at a level at least 50%, 100%, 150% or 200% compared to CMV promoter (SEQ ID NO: 64) in muscle stem cell derived cells (e.g. c2c12 or H2K cells (skeletal muscle)).

Selecting a MuSC-RCs for use in a vector for treating different neuromuscular disease (NMD) as disclosed herein can be optimized using a multistep process, that will differ for each disease, and possibly among different alleles for each disease. This is due to the fact that each NMD affects a different protein, and the normal concentrations of these proteins may differ by 3 or more orders of magnitude. Moreover, since each mRNA and its encoded protein have different intrinsic half-lives, the amounts of mRNA required to produce normal amounts of each therapeutic protein will differ for each NMD.

A further complexity is the possibility that different disease gene alleles among different patients with the same NMD may produce non-functional or poorly functioning mutant proteins that compete with the therapeutic protein for binding to partner proteins, thereby necessitating higher levels of the therapeutic protein than found in normal muscle cells in order to “outcompete” the deleterious protein. An additional complexity is that until biomarker or functional improvement assays demonstrate efficacy in pilot studies that test graded vector doses or activity levels, it's challenging to predict how much therapeutic product is needed for optimal benefits. In this regard, it's important to recognize that while suboptimal therapeutic protein levels can be overcome via graded vector dose and MuSC-RC activity increases, toxic levels will not necessarily be detected by the same biomarker or functional assays. Rather, toxicity may only be detected via assays that focus on predicted problems that could be logically associated with excess therapeutic product levels. Furthermore, these toxicity phenotypes may require extended time periods to be observed. Patient safety is thus at risk by assuming that high levels of a therapeutic protein will be inconsequential simply because functional benefits are observed. This, the ability to titrate expression using cassettes with different activities can be important, and the cassettes described herein permit such titration.

Step-1. MuSC Selection Based on cDNA Size.

Stepwise strategies for identifying optimal MuSC-RCs can begin with determining the size of the therapeutic product's cDNA, including the size of any 5′- and/or 3′-untranslated regions, as well as introns that may be necessary for obtaining sufficient levels of the functional protein. This information, together with the packaging size limits of the vector can then be used to determine the size range of MuSC-RCs that can be efficiently packaged with the particular cDNA.

For AAV vectors that have a 4.8 kb efficient packaging limit, and that require the presence of two Inverted Terminal Repeat (ITR) sequences of about 145 bases for genomic packaging, the combined MuSC-RC plus cDNA size limit is 4.5 kb. Thus, for cDNAs smaller than 0.5, 1, 2, 3, and 4 kb, the compatible MuSC-RC sizes would need to be less than 4, 3.5, 2.5, 1.5, and 0.5 kb. Since many MuSC-RCs have been purposely miniaturized so as to be compatible with packaging larger cDNAs, this means that multiple MuSC-RCs are available for expressing almost all NMD proteins. Use of MuSC-RCs for expressing even larger proteins is not, however, precluded, because AAV vectors can package constructs as large as 5.2 kb with much lower efficiencies, and also because cDNAs exceeding 10 kb can be “packaged” as fragments that can recombine following transduction. In some embodiments, the MuSC-RC can be used in a gene expression or gene editing system comprising multiple expression parts, such as split-proteins or split inteins, for example, as disclosed in WO 2023/004125, which is incorporated herein in its entirety by reference.

### Step-2. MuSC-RC Selection Based on Specific Muscle Stem Cell Expression.

The second step in identifying the most appropriate MuSC-RC is identification that the transgene is expressed predominantly in skeletal muscle stem cells, and not in differentiated or mature skeletal muscle cells. Further narrowing of MuSC-RC choices would come from knowledge regarding which human skeletal muscles are most seriously affected by the disease (e.g., Duchenne Muscular Dystrophy affects essentially all striated muscles, while Limb Girdle Muscular Dystrophies affect only a subset of anatomical muscles, and Facioscapulohumeral Muscular Dystrophy affects a predominantly different muscle group). Similarly, some NMDs have greater relative effects among certain human skeletal muscle fiber types: I, IIa, and IIx. One selection can be based on expression in skeletal muscle stem cells vs expression in cardiac muscle.

### Step-3. MuSC Selection Based on Functional Product Concentration Levels.

After narrowing MuSC-RC choices based on the previous two criteria, it's helpful to estimate the concentration of therapeutic protein required to provide functional benefits for the particular NMD; and, if data are available, to know whether excessive levels of the therapeutic protein may be toxic.

Importantly, as the MuSC-RC are active in muscle stem cells, and in the case of gene editing, which benefits from expression of the gene editing machinery for a transient period necessary for the gene editing to occur, maximal levels of transgene expression are not necessarily required; rather, it is the specificity and selectively targeting the muscle stem cells as compared to non-stem muscle cells which are important criteria. This provides a more informative strategy for identifying MuSC-RC with optimal transcriptional activities in muscle stem cells of mRNA at a level sufficient for the gene editing to occur, or expression of the transgene to occur, when the muscle stem cells either differentiate to muscle cells, or regenerate. Selection of these MuSC-RCs can be done based on the transcriptional activity selectively in muscle stem cells in conjunction with the packaging size and expression level, and this can be compared to that of the native M-creatine kinase (CKM) enhancer-promoter, or CK8e regulatory element comprising SEQ ID NO: 306.

### VI. Vectors and Expression Cassettes

### A. MuSC-RC for Gene-Editing:

The MuSC-RCs as disclosed herein can be used in vectors for gene editing in muscle stem cells, or satellite cells. Accordingly, one aspect of the technology described herein relates to use of MuSC-RCs as disclosed herein for gene editing that uniquely permits genomic modifications in MuSCs to be inherited by resulting progeny that either differentiate to repair injured muscle, or return to quiescence as MuSCs for future regenerative needs.

In vivo gene transfer therapies, including CRISPR/Cas or other nucleic acid-guided nuclease-based editing approaches require the delivery of enzyme and guide RNA payloads of varying sizes, and the size limitations of AAV vectors presents an ongoing challenge for those requiring larger payloads. Split inteins provide one approach for the introduction of larger proteins to target cells, but approaches that minimize the size of vector regulatory sequences while maintaining cell-type specificity can expand the range of gene editing machinery sizes and target specificities. Thus, reduced-size, high specificity regulatory constructs as described herein, and/or a combination of such reduced size, high specificity or cell-type-restricted expression cassettes with the split intein approach provides increased flexibility for the delivery of gene editing components using, for example, AAV vectors. Based on the cDNA size requirements of, for example, smaller editing systems delivered as single vectors (i.e. SaCas9 or Cpf-1), or larger split-vector systems (SpCas9, epigenetic modifiers, transcriptional repressors/activators, as well as base- & prime-editors) the MuSC-RC disclosed herein can be optimized for a wide range sizes. Depending on the size requirements of the intended gene delivery system, the MuSC-RC may be used to build expression vectors with ideal transcriptional activities.

In some embodiments, the MuSC-RC can be used for gene editing for Duchenne muscular dystrophy (DMD). In some embodiments, the MuSC-RC are used in methods, compositions and systems for treating DMD as disclosed, for example, in US Application US2017/0362635, which is incorporated herein in its entirety by reference.

Without wishing to be bound by theory, gene replacement therapies utilizing adeno-associated viral (AAV) vectors hold promise for treating Duchenne muscular dystrophy (DMD). A potentially longer-lasting approach revolves around efforts to directly modify the dystrophin gene using the CRISPR/Cas9 system. Here, the MuSC-RC can be used for editing the genes in muscle stem cells, using both single- and dual-AAV vector delivery of a Cas9 cassette operatively linked to a MuSC-RC together with single-guide RNA cassettes and, in one approach, a dystrophin homology region. Cas9 expression specifically in muscle stem cells can lead to direct gene editing of the mutation, multi-exon deletion or complete gene correction via homologous recombination, which, when the muscle stem cells differentiate, will be reproduced in post-mitotic myofibers.

Induction of dystrophin expression was tested following AAV6-mediated delivery of CRISPR/Cas9 components derived from either Streptococcus pyogenes (SpCas9) (see Cong, L., et al. Science 339, 819-823 (2013)) or Staphylococcus aureus (SaCas9) (see Ran, F. A., et al. Nature 520, 186-191 (2015)) using dual- or single-vector approaches, respectively. Previous reports have used a muscle-specific CK8 regulatory cassette (RC) to restrict Cas9 expression in skeletal and cardiac muscle (see Himeda, C. L., et al. Methods Mol. Biol. 709, 3-19 (2011)) in order to reduce the risk of off-target events in non-muscle cells and to minimize elicitation of an immune response (see Hartigan-O'Connor, D., et al. Mol. Ther. 4, 525-533 (2001) and Hu, C., et al. Mol. Ther. 22, 1792-1802 (2014)). Several approaches were also previously tested to either excise exons 52 and 53 (Δ5253; strategy 1) or to directly target the mutation in exon 53 (53*; strategy 2). Due to the ~5 kb packaging limit of AAV, dual-AAV vectors were used in tandem: a nuclease vector expressing SpCas9 under control of the CK8 RC and a set of targeting vectors containing two single-guide RNA (sgRNA) expression cassettes, or an alternative strategy using CK8-regulated expression of the smaller SaCas9 enabled use of a single vector.

Accordingly, in some embodiments, a MuSC-RC as disclosed herein can be used in vectors, e.g., viral vectors such as AAV vectors for gene editing for the treatment of DMD.

Accordingly, without wishing to be bound by theory, the MuSC-RC can be used in one or more vectors for gene editing. In some embodiments, a MuSC-RC as disclosed herein can be used in a construct that comprises any one or more of the following: one or more guide RNAs (gRNA) (e.g., a first gRNA cassette); a mutation-corrected homology template (e.g., for HDR); and a nuclease cassette. In some embodiments, a vector can comprise a MuSC-RC operatively linked to a nuclease coding sequence. In some embodiments, the nuclease coding sequence may encode a CRISPR-associated nuclease. For example, the nuclease coding sequence may encode a protein selected from SaCas9, SpCas9, Cpf1, or another suitable CRISPR-associated nuclease.

In some embodiments, a vector can comprise a MuSC-RC operatively linked a second gRNA cassette, wherein the first gRNA cassette includes a first gRNA coding sequence and the second gRNA cassette includes a second gRNA coding sequence. In some other embodiments, the pharmaceutical composition may further include three or more gRNA cassettes. For example, the pharmaceutical composition may further include: a third gRNA cassette, wherein the third gRNA cassette includes a third gRNA coding sequence; a fourth gRNA cassette, wherein the fourth gRNA cassette includes a fourth gRNA coding sequence; and so on.

In certain embodiments, a vector can comprise a MuSC-RC operatively linked to a mutation-corrected DNA template, wherein the mutation-corrected DNA template is configured for HDR. The MuSC-RC and/or the gRNA cassettes described above may also include such a mutation-corrected DNA template (or the mutation-corrected DNA template may be delivered separately from the muscle-specific transcriptional regulatory cassette and/or the gRNA cassettes), wherein the mutation-corrected DNA template may be configured for HDR. The mutation-corrected DNA template may be configured to repair a mutated target nucleic acid sequence. In some embodiments, the mutated target nucleic acid sequence may be in a gene associated with a neuromuscular disorder. For example, the mutated target nucleic acid sequence may be in a gene encoding dystrophin.

In some embodiments, a vector comprising a MuSC-RC as disclosed herein for gene editing is a recombinant adeno-associated virus (rAAV) vector. For example, the rAAV vector may be an rAAV6 vector, an rAAV8, an rAAV9 vector, or another suitable rAAV vector. In various embodiments, the rAAV vector may be an rAAV6 vector. The delivery system may include a single rAAV vector to deliver the muscle-stem cell specific nuclease cassette and the one or more gRNA cassettes. Alternatively, the delivery system may include a first rAAV vector to deliver the muscle-stem cell specific nuclease cassette and a second rAAV vector to deliver the one or more gRNA cassettes. Furthermore, the delivery system may include a third rAAV vector to deliver an additional gRNA cassette, a fourth rAAV vector to deliver an additional gRNA cassette, and so on. Any of these rAAV vectors may include a mutation-corrected DNA template configured for HDR.

Accordingly, in some embodiments a vector comprising a MuSC-RC as disclosed herein for gene therapy is present in a pharmaceutical composition. In some embodiments, a vector comprising a MuSC-RC as disclosed herein may reduce a pathological effect or symptom of a neuromuscular disorder in a subject.

In various embodiments, a vector comprising a MuSC-RC as disclosed herein can be used to regulate the expression of a transgene in a muscle stem cell, which, depending on the transgene delivered, may ultimately result in an increase a specific-force generating capacity of at least one skeletal muscle in a subject to within at least 25%, at least 30%, at least 40%, or at least 50% of a normal specific-force generating capacity in a skeletal muscle. In some embodiments, a vector comprising a MuSC-RC as disclosed herein can be used to restore a baseline end-diastolic volume defect in a subject to within at least 25%, at least 30%, at least 40%, or at least 50% of a normal end-diastolic volume.

Accordingly, one aspect of the disclosure relates to methods of modifying the sequence of a target nucleic acid sequence in a muscle stem cell or a myogenic progenitor cell. In certain embodiments, the method may include contacting or transducing the muscle stem cell or progenitor cell with one or more vectors, e.g., an AAV vector as disclosed herein, where the one or more vectors may include a muscle-specific nuclease cassette, one or more gRNA cassettes, and/or a mutation-corrected DNA template comprising a modification to be made in the target nucleic acid sequence (i.e., a homology template for HDR).

In some embodiments, the MuSC-RC can be used in any targeted genetic engineering approach. The CRISPR (Clustered Regularly Interspaced Short Palindromic Repeats)/Cas (CRISPR-associated protein) nuclease system is an engineered nuclease system used for genetic engineering that is based on a bacterial system. Information regarding CRISPR-Cas systems and components thereof are described in, for example, U.S. Pat. Nos. 8,697,359, 8,771,945, 8,795,965, 8,865,406, 8,871,445, 8,889,356, 8,889,418, 8,895,308, 8,906,616, 8,932,814, 8,945,839, 8,993,233 and 8,999,641 and applications related thereto; and WO2014/018423, WO2014/093595, WO2014/093622, WO2014/093635, WO2014/093655, WO2014/093661, WO2014/093694, WO2014/093701, WO2014/093709, WO2014/093712, WO2014/093718, WO2014/145599, WO2014/204723, WO2014/204724, WO2014/204725, WO2014/204726, WO2014/204727, WO2014/204728, WO2014/204729, WO2015/065964, WO2015/089351, WO2015/089354, WO2015/089364, WO2015/089419, WO2015/089427, WO2015/089462, WO2015/089465, WO2015/089473 and WO2015/089486, WO2016205711, WO2017/106657, WO2017/127807 and applications related thereto.

Zinc finger nucleases (ZFNs) can also be used for gene editing and can be delivered using vectors comprising MuSCs-RCs as described herein. ZFNs are described in, e.g., U.S. Pat. Nos. 6,534,261; 6,607,882; 6,746,838; 6,794,136; 6,824,978; 6,866,997; 6,933,113; 6,979,539; 7,013,219; 7,030,215; 7,220,719; 7,241,573; 7,241,574; 7,585,849; 7,595,376; 6,903,185; 6,479,626; US 2003/0232410 and US 2009/0203140 as well as Gaj et al., Nat Methods, 2012, 9(8):805-7; Ramirez et al., Nucl Acids Res, 2012, 40(12):5560-8; Kim et al., Genome Res, 2012, 22(7): 1327-33; Urnov et al., Nature Reviews Genetics, 2010, 11:636-646; Miller, et al. Nature biotechnology 25, 778-785 (2007); Bibikova, et al. Science 300, 764 (2003); Bibikova, et al. Genetics 161, 1169-1175 (2002); Wolfe, et al. Annual review of biophysics and biomolecular structure 29, 183-212 (2000); Kim, et al. Proceedings of the National Academy of Sciences of the United States of America 93, 1156-1160 (1996); and Miller, et al. The EMBO journal 4, 1609-1614 (1985).

Transcription activator like effector nucleases (TALENs) can also be used for gene editing and can be delivered using vectors comprising MuSCs-RCs as described herein. TALENs are described in U.S. Pat. Nos. 8,440,431; 8,440,432; 8,450,471; 8,586,363; and 8,697,853; as well as Joung and Sander, Nat Rev Mol Cell Biol, 2013, 14(1):49-55; Beurdeley et al., Nat Commun, 2013, 4: 1762; Scharenberg et al., Curr Gene Ther, 2013, 13(4):291-303; Gaj et al., Nat Methods, 2012, 9(8):805-7; Miller, et al. Nature biotechnology 29, 143-148 (2011); Christian, et al. Genetics 186, 757-761 (2010); Boch, et al. Science 326, 1509-1512 (2009); and Moscou, & Bogdanove, Science 326, 1501 (2009).

### B. MuSC-RC for Gene Expression of Therapeutic Transgenes:

In a further aspect of the technology described herein, there is provided an expression cassette comprising a synthetic MuSC-RC as described herein, operably linked to a sequence encoding an expression product, suitably a gene, e.g. a transgene. In some embodiments the expression product is a therapeutic expression product.

Many clinical uses for MuSC-RCs will be for expression of transgenes for neuromuscular disease gene therapies and/or CRISPR/Cas9 gene correction strategies in which their high muscle specificity & ability to modify muscle stem cells that generate skeletal muscle provides beneficial safety features. In cases where very high product levels are needed, the most active MuSC-RCs will facilitate treatments at lower vector doses that are safer & more economical. MuSC-RCs are also applicable for controlling gene expression of a transgene for any therapy in which muscle stem cells could serve to regenerate muscles, and thus provide a renewable in vivo source of secreted proteins (i.e., regenerative muscles express the transgene); e.g., hormones, clotting factors, antibodies & enzymes as an alternative to intravenous protein replacement therapies, as well as secreted metabolites. MuSC-RCs could also be used to control gene expression as potentially safer expression cassettes than viral promoters for DNA-based immunization strategies.

The therapeutic expression product may be a nucleic acid that increases expression of an endogenous nucleic acid that encodes a protein, or may be a nucleic acid that can provide a nucleic acid modulator of gene expression such as an siRNA.

The therapeutic expression product may be an angiogenic protein. Angiogenic proteins promote development and differentiation of blood vessels. Examples of angiogenic proteins include members of the fibroblast growth factor (FGF) family such as aFGF (FGF-1), bFGF (FGF-2), FGF-4 (also known as “hst/KS3”), FGF-5 and FGF-6, the vascular endothelial growth factor (VEGF) family, the platelet-derived growth factor (PDGF) family, the insulin-like growth factor (IGF) family, and others.

### C. Vectors Comprising the MuSC-RC

In a further aspect, there is provided a vector comprising a synthetic muscle-stem cell regulatory cassette (MuSC-RC) or an expression cassette according to the present invention. In some embodiments the vector is an expression vector. In some embodiments the vector is a viral vector. In some embodiments the vector is a gene therapy vector, suitably an AAV vector, an adenoviral vector, a retroviral vector or a lentiviral vector. AAV vectors are of particular interest. AAV vectors may be selected, for example, from the group consisting of AAV2, AAV6, AAV8, AAV9, BNP116, rh10, AAV2.5, AAV2i8, AAVDJ8 and AAV2G9, or derivatives thereof. AAV serotype 9 (AAV9) has been noted to achieve efficient transduction in skeletal muscle, and thus AAV9 and derivatives thereof represent one non-limiting example of a suitable AAV vector. In some embodiments, the rAAV vector is a AAV3b serotype, including, but not limited to, an AAV3b265D virion, an AAV3b265D549A virion, an AAV3b549A virion, an AAV3bQ263Y virion, or an AAV3bSASTG virion (i.e., a virion comprising a AAV3b capsid comprising Q263A/T265 mutations). In some embodiments, the virion can be rational haploid, or a chimeric or any mutant, such as capsids tailored for increased uptake at a desired location, e.g., the heart or skeletal muscle. Other capsids can include capsids from any of the known AAV serotypes, including AAV1, AAV3, AAV4, AAV5, AAV7, AAV10, etc. In some preferred embodiments, the AAV vector is AAV2i8.

In some embodiments, the technology relates to an expression vector comprising a MuSC-RC as disclosed herein, where the expression vector is a viral vector, such as an adeno-associated viral vector, e.g. an AAV6, AAV2, rAAV2/1, rAAV2/2, rAAV2/3, rAAV2/4, rAAV2/5, rAAV2/6, rAAV2/7 rAAV2/8, rAAV2/9, rAAV2/10, rAAVM41, dsAAV, etc. In one embodiment of any of the aspects described herein, the adeno-associated viral vector is selected from the group consisting of: an AAVRh74 vector, an AAV8 vector, an AAV9 vector, an AAV6 vector, an AAV7 vector, an AAV2i8 vector, a NP vector, a NP 66 vector, a NP 22 vector, an AAVpo.1 vector, a MyoAAV vector, and an AAVMyo vector.

In various embodiments, the expression vector further comprises a transduction reporter.

The vector according to the present invention may be an AAV vector comprising a MuSC-RC operatively linked to a nucleic acid encoding a therapeutic expression product, as disclosed herein. In a further aspect, there is provided a virion (viral particle) comprising a vector, suitably a viral vector, comprising a MuSC-RC according to the present invention. In some embodiments the virion is an AAV virion. In a further aspect, there is provided a pharmaceutical composition comprising a MuSC-RC, vector or virion according to the present invention.

In a further aspect, there is provided a synthetic MuSC-RC, expression cassette, vector, virion or pharmaceutical composition according to the present invention for use in therapy, i.e. the prevention or treatment of a medical condition or disease, suitably for use in therapy of a subject in need thereof. Suitably the condition or disease is associated with aberrant gene expression, e.g., aberrant gene expression in muscle cells (myocytes) or tissue. Suitably the condition or disease is associated with aberrant gene expression in skeletal muscle or tissue.

### VII. Use of the SCRC's in Methods of Treatment of Muscle Disease or Disorders

As disclosed herein, the MuSC-RC can be used in vectors for the treatment of a muscle disease or disorder, or neuromuscular disorder. In some embodiments, the pathological effect or symptom of the neuromuscular disorder may be selected from at least one of muscle pain, muscle weakness, muscle fatigue, muscle atrophy, fibrosis, adipose cell accumulation, inflammation, increase or decrease in average myofiber diameter in skeletal muscle, or centrally-nucleated myofiber number, cardiomyopathy, reduced 6-minute walk test time, loss of ambulation, and cardiac pump failure.

The neuromuscular disorder may be a muscular dystrophy selected from at least one of myotonic muscular dystrophy (DM1 and/or DM2), Duchenne muscular dystrophy, Becker muscular dystrophy, any of the various types of limb-girdle muscular dystrophy, facioscapulohumeral muscular dystrophy, any of the various types of congenital muscular dystrophy, oculopharyngeal muscular dystrophy, distal muscular dystrophy, desmin-related myopathies, fukyama muscular dystrophy, FKRP-deficiencies and Emery-Dreifuss muscular dystrophy. In some embodiments, the muscular dystrophy may be Duchenne muscular dystrophy.

Other disorders include the treatment of neuromuscular/neuromotor disorders such as spasticity, amyotrophic lateral sclerosis, and dystonia.

Another aspect of the disclosure relates to methods for treating a subject having a neuromuscular disorder. In certain embodiments, the method may include administering to the subject a therapeutically effective amount of a pharmaceutical composition. The pharmaceutical composition may include a MuSC-RC operatively linked to a nuclease and a vector comprising a MuSC-RC operatively linked to one or more gRNA cassettes and/or a mutation corrected template for HDR. The pharmaceutical composition may further include a delivery system for delivery of the muscle-specific nuclease cassette, the one or more gRNA cassettes, and/or the mutation-corrected DNA template configured for HDR.

Methods disclosed herein include treating subjects (e.g., humans, veterinary animals (dogs, cats, reptiles, birds) livestock (e.g., horses, cattle, goats, pigs, chickens) and research animals (e.g., monkeys, rats, mice, fish) with compositions disclosed herein. Treating subjects includes delivering therapeutically effective amounts. Therapeutically effective amounts include those that provide effective amounts for prophylactic treatments and/or therapeutic treatments.

Skeletal and cardiac muscles are affected by hundreds of genetic diseases, are subject to many types of physical injury, undergo progressive functional weakness during disuse and aging, and skeletal muscle also undergoes debilitating catabolic degradation in conjunction with cancers. Since all skeletal muscle fibers are innervated, and since the function of muscle cell synaptic regions where neuronal axons stimulate muscle contraction depends on neuronal interactions, muscle cells also exhibit a variety of Neuromuscular Junction (NMJ) diseases. Additionally, since the maintenance of innervating neurons is partially dependent on muscle-mediated signals, muscles also play important roles in the normal function of their innervating neurons. The appropriate regulatory cassettes can play major roles in therapeutic strategies for combating all of these medical issues, as well as analogous issues in veterinary medicine.

Age-related sarcopenia, cancer cachexia, and spinal cord injuries tend to affect Type II fibers more than Type I fibers. Duchenne (DMD) and Facioscapulohumeral (FSHD) muscular dystrophies also exhibit greater effects on Type II fibers, while FKRP-mediated Dystroglycanopathies (MDDGA5, MDDGB5 & MDDGC5), exhibit relative increases in Type I fibers, possibly due to gradual Type II- to Type I transitions. In contrast, Myotonic dystrophy and some Limb Girdle Muscular Dystrophies (e.g., LGMD2A due to Calpain-3 deficiency) are associated with reduced Type I fibers. NMJ diseases also exhibit skeletal muscle fiber type changes; e.g., infants with the most severe forms of Spinal Muscular Atrophy (SMA) have many fewer Type II fibers and an associated increase in Type I fibers; and patients with advanced Amyotrophic Lateral Sclerosis (ALS) exhibit a transition from Type II to Type I fibers. Some striated muscle diseases such as DMD affect both skeletal and cardiac muscles, whereas others primarily affect skeletal or cardiac muscle, and some cardiac muscle diseases have their most pronounced effects on either ventricular, atrial, or conduction components. These disease-specific muscle type differences underscore the importance of regulatory cassettes that are optimized for each disease type so as to focus gene therapies to the most affected muscles and fiber types.

Particular muscle-related disorders that can be treated include cardiac muscle disease (e.g., Hypertrophic Cardiomyopathy) and Striated muscle diseases including dystrophies and dystroglycanopathies. Dystrophies include muscular dystrophies and Myotonic dystrophies. Examples of muscular dystrophies include Limb-girdle muscular dystrophies (LGMD), LGMD2A due to calpain-3 deficiency, MTM1, ACTA1 LGMD, Duchenne Muscular Dystrophy (DMD), and Facioscapulohumeral muscular dystrophy (FSHD). Examples of Dystroglycanopathies include MDC1A, MDDGA5, MDDGB5, MDDGC5, and MDDGC14 (GMPPB disease). Neuromuscular disorders (NMD) and Neuromuscular junction disorders (NMJ) can also be treated. These include Spinal Muscular Atrophy (SMA), amyotrophic lateral sclerosis (ALS), and Myasthenic NMDs (e.g., Congenital myasthenic (those affecting acetylcholine receptor subunits (CHRNA1; CHRNB1; CHRND; CHRNE), COLQ or DOK7), LGMD2A and MTM1. Additional examples of disorders that can be treated include amyopathies, Nemalin myopathies (e.g., Nemaline Myopathy-2), Myofibrillar Myopathy-5, Miyoshi Myopathy, Scapuloperoneal Myopathy, X-linked myotubular myopathy, Central Core Disease, Paramyotonia, Pompe Disease, Cancer cachexia, and aging diseases (age-related sarcopenia), among other diseases or disorders described elsewhere herein.

In particular embodiments, therapeutically effective amounts provide anti-muscle-related disorder effects. Anti-muscle-related disorder effects can one or more of: maintain or increase muscular health, maintain or increase muscle strength, reduce or resolve muscle injury, reduce or resolve muscle atrophy or loss, or reduce or resolve cancer- or infection-related cachexia. In particular embodiments, therapeutically effective amounts result in muscle regeneration.

For administration, therapeutically effective amounts (also referred to herein as doses) can be initially estimated based on results from in vitro assays and/or animal model studies. Such information can be used to more accurately determine useful doses in subjects of interest.

The actual dose amount administered to a particular subject can be determined by a physician, veterinarian or researcher taking into account parameters such as physical and physiological factors including target, body weight, severity of condition, previous or concurrent therapeutic interventions, idiopathy of the subject and route of administration.

Therapeutically effective amounts can be achieved by administering single or multiple doses during the course of a treatment regimen (e.g., daily, every other day, every 3 days, every 4 days, every 5 days, every 6 days, weekly, every 2 weeks, every 3 weeks, monthly, every 2 months, every 3 months, every 4 months, every 5 months, every 6 months, every 7 months, every 8 months, every 9 months, every 10 months, every 11 months or yearly).

The pharmaceutical compositions described herein can be administered by, for example, injection, inhalation, infusion, perfusion, lavage, or ingestion. Routes of administration can include intravenous, intradermal, intraarterial, intraparenteral, intranasal, intralesional, intramuscular, oral, subcutaneous, and/or sublingual administration

Compositions. Vectors described herein can be formulated into compositions for administration to subjects. Compositions include a therapeutically effective amount of one or more vectors and a pharmaceutically acceptable carrier.

Exemplary generally used pharmaceutically acceptable carriers include any and all absorption delaying agents, antioxidants, binders, buffering agents, bulking agents or fillers, chelating agents, coatings, disintegration agents, dispersion media, gels, isotonic agents, lubricants, preservatives, salts, solvents or co-solvents, stabilizers, surfactants, and/or delivery vehicles. Exemplary antioxidants include ascorbic acid, methionine, and vitamin E. Exemplary buffering agents include citrate buffers, succinate buffers, tartrate buffers, fumarate buffers, gluconate buffers, oxalate buffers, lactate buffers, acetate buffers, phosphate buffers, histidine buffers, and/or trimethylamine salts. An exemplary chelating agent is EDTA (ethylene-diamine-tetra-acetic acid). Exemplary isotonic agents include polyhydric sugar alcohols including trihydric or higher sugar alcohols, such as glycerin, erythritol, arabitol, xylitol, sorbitol, or mannitol. Exemplary preservatives include phenol, benzyl alcohol, meta-cresol, methyl paraben, propyl paraben, octadecyldimethylbenzyl ammonium chloride, and benzalkonium halides.

Stabilizers refer to a broad category of excipients which can range in function from a bulking agent to an additive which solubilizes the antibodies or helps to prevent denaturation or adherence to the container wall. Typical stabilizers can include polyhydric sugar alcohols, amino acids, organic sugars or sugar alcohols, PEG, amino acid polymers, sulfur-containing reducing agents, low molecular weight polypeptides (i.e., <10 residues), proteins such as human serum albumin, bovine serum albumin, gelatin or immunoglobulins, hydrophilic polymers, monosaccharides, disaccharides, trisaccharides, and polysaccharides.

The compositions disclosed herein can be formulated for administration by, for example, injection, inhalation, infusion, perfusion, lavage, or ingestion. The compositions disclosed herein can further be formulated for intravenous, intradermal, intraarterial, intranodal, intralymphatic, intraperitoneal, intralesional, intraprostatic, intravaginal, intrarectal, topical, intrathecal, intratumoral, intramuscular, intravesicular, oral, sublingual, and/or subcutaneous administration. For injection, compositions can be formulated as aqueous solutions, such as in buffers including Hanks' solution, Ringer's solution, or physiological saline. The aqueous solutions can include formulatory agents such as suspending, stabilizing, and/or dispersing agents. Alternatively, the formulation can be in lyophilized and/or powder form for constitution with a suitable vehicle, e.g., sterile pyrogen-free water, before use.

While gene therapy vectors are more commonly delivered parenterally, oral administration is contemplated. For oral administration, the compositions can be formulated as tablets, pills, dragees, capsules, liquids, gels, syrups, slurries, suspensions and the like.

Compositions can be formulated as an aerosol. In particular embodiments, the aerosol is provided as part of an anhydrous, liquid or dry powder inhaler. Aerosol sprays from pressurized packs or nebulizers can also be used with a suitable propellant, e.g., dichlorodifluoromethane, trichlorofluoromethane, dichlorotetrafluoroethane, carbon dioxide or other suitable gas. Compositions can also be formulated as depot preparations. Depot preparations can be formulated with suitable polymeric or hydrophobic materials (for example as an emulsion in an acceptable oil) or ion exchange resins, or as sparingly soluble derivatives, for example, as a sparingly soluble salt.

Any composition disclosed herein can advantageously include other pharmaceutically acceptable carriers which include those that do not produce significantly adverse, allergic, or other untoward reactions that outweigh the benefit of administration. Exemplary pharmaceutically acceptable carriers and formulations are disclosed in Remington's Pharmaceutical Sciences, 18th Ed. Mack Printing Company, 1990. Moreover, formulations can be prepared to meet sterility, pyrogenicity, general safety, and purity standards as required by U.S. FDA Office of Biological Standards and/or other relevant foreign regulatory agencies.

In various embodiments, the therapeutically effective amount of the pharmaceutical composition may be between about 10¹¹ and about 10¹⁶ vector genomes (vg)/kilogram (kg) subject weight, between about 10¹² and about 10¹⁵ vg/kg subject weight, between about 10¹³ and about 10¹⁴ vg/kg subject weight, or another suitable amount. In some embodiments, the pharmaceutical composition may be administered intravascularly, intraperitoneally, subcutaneously, or orally. In certain embodiments, the pharmaceutical composition may include no, up to 5%, up to 10%, up to 20%, up to 30%, up to 40%, up to 50%, up to 60%, up to 70%, up to 80%, or up to 90% empty capsids (see U.S. Pat. No. 7,655,467 and European Patent No. 1689230).

Another aspect of the disclosure relates to methods of using MuSC-RC in vectors for modifying a sequence of a target nucleic acid sequence in a muscle stem cell or a myogenic progenitor cell. In certain embodiments, the method may include contacting or transducing the muscle stem cell, satellite cell or the myogenic progenitor cell with a delivery system and/or the contents of the delivery system. As disclosed herein, the delivery system may include one or more vectors (e.g., viral vectors, e.g., AAV vectors), comprising a MuSC-RC as disclosed herein, operatively linked to any one or more of: a nuclease cassette, one or more gRNA cassettes, and/or a mutation-corrected DNA template comprising a modification to be made in the target nucleic acid sequence (i.e., a homology template for HDR).

In a further aspect, there is provided a muscle stem cell comprising a synthetic MuSC-RC, expression cassette, vector, or virion of the present invention. In some embodiments the cell is a eukaryotic cell, optionally a mammalian cell, optionally a human cell. Suitably the cell can be a muscle stem cell, optionally, a human muscle stem cell. Suitably the cell can be a human skeletal muscle stem cell. The synthetic muscle stem cell specific expression cassette or construct can be episomal or can be in the genome of the cell.

In a further aspect, there is provided a synthetic MuSC-RC, or expression cassette, vector, virion comprising the same, or pharmaceutical composition as described herein for use in the manufacture of a pharmaceutical composition for the treatment of a medical condition or disease.

In a further aspect, there is provided a method for producing an expression product, the method comprising providing a synthetic MuSC-RC of the present invention in a muscle cell or muscle stem cell and expressing the gene operatively linked to the MuSC-RC. The method can be in vitro or ex vivo, or it can be in vivo. In some embodiments the method is a bioprocessing method. In one embodiment, the muscle stem cell is a skeletal muscle stem cell.

In a further aspect, there is provided a method of expressing a therapeutic transgene in a muscle stem cell, the method comprising introducing into the muscle stem cell a MuSC-RC, where the MuSC-RC is operatively linked to the therapeutic transgene and is present in a vector or virion as described herein. In one embodiment, the muscle stem cell is a skeletal muscle cell.

In a further aspect, there is provided a method of therapy of a subject, preferably a human, in need thereof, the method comprising: administering to the subject a cassette comprising a MuSC-RC operatively linked to a transgene, where the cassette is present in a vector, virion or pharmaceutical composition as described herein, where the transgene comprises a sequence encoding a therapeutic product operably linked to a MuSC-RC according to the present invention; and expressing a therapeutic amount of the therapeutic product in the muscle stem cell of said subject.

In one embodiment, the muscle stem cell is a skeletal muscle stem cell. In some embodiments, the muscle stem cell is not a cardiac muscle cell.

In some embodiments the method comprises: introducing into the muscle of the subject an expression cassette, vector, virion or pharmaceutical composition comprising a MuSC-RC operatively linked to a transgene as described herein, where the transgene comprises a gene encoding a therapeutic product; and expressing a therapeutic amount of the therapeutic product in the muscle stem cell of said subject. In one embodiment, the muscle stem cell is a skeletal muscle stem cell. In some embodiments, the muscle stem cell is not a cardiac muscle cell.

Suitably the method comprises administering a vector, virion or pharmaceutical composition as described herein to the subject. In some preferred embodiments the vector is a viral gene therapy vector, preferably an AAV vector.

Further features and embodiments of the present invention will now be described under the following sections. Any feature or embodiment in any section may be combined with any other feature or embodiment, or with any aspect of the invention, in any workable combination.

### X. Paragraph PCT

Some embodiments of the technology described herein can be defined according to any of the following numbered paragraphs:
1. A muscle-stem cell specific regulatory nucleic acid cassette (MuSC-RC) for selectively regulating the expression of an operatively linked heterologous transgene in a muscle stem cell (mSC), the MuSC-RC comprising at least two regulatory elements (RE), wherein the two RE's are selected from:
   (i) at least two REs located in an untranslated region of the PAX7 gene,
   (ii) at least two REs located in an untranslated region in the MyoD1 gene,
   (iii) at least one RE located in an untranslated region of the PAX7 gene, and at least one RE located in an untranslated region in the MyoD1 gene,
   wherein the two RE are located adjacent to each other and are recombinant with respect to each other.
2. The MuSC-RC of paragraph 1, wherein the muscle stem cell (mSC) is a skeletal muscle satellite cell.
3. The MuSC-RC of paragraph 1, comprising:
   a. a nucleic acid sequence comprising at least one regulatory element (RE) from the untranslated region of the human PAX7 gene, or a functional fragment thereof, and
   b. a nucleic acid sequence comprising at least one regulatory element (RE) from the untranslated region of the human of MYOD1 gene, or a functional fragment thereof.
4. The MuSC-RC of paragraph 1, comprising:
   a. a nucleic acid sequence comprising at least two regulatory elements (RE) from the 3′ UTR of the human PAX7 gene, or a functional fragment thereof, or
   b. a nucleic acid sequence comprising at least two regulatory elements (RE) from the 5′ UTR of the human of MYOD1 gene, or a functional fragment thereof, or
   c. a nucleic acid sequence comprising at least one regulatory element (RE) from the 5′ UTR of the human PAX7 gene, or a functional fragment thereof, and at least one regulatory element (RE) from the 5′ UTR of the human MyoD1 gene.
5. The MuSC-RC of any of paragraphs 3-4, wherein a RE from the human PAX7 gene is selected from any of: SEQ ID NO: 1-16, or a nucleic acid sequence having at least 85% sequence identity thereto.
6. The MuSC-RC of any of paragraphs 3-4, wherein a RE from the human MyoD1 gene is selected from any of: SEQ ID NO: 17-22, or a nucleic acid sequence having at least 85% sequence identity thereto.
7. The MuSC-RC of any of paragraphs 1-6, comprising any one of:
   a. a nucleic acid sequence comprising SEQ ID NO: 4 and SEQ ID NO: 5 (R4-Px7 and R5-Px7 from the human PAX7 gene), or a functional fragment thereof, or a sequence having at least 85% sequence identity to at least SEQ ID NO: 4 or SEQ ID NO:5 or a functional variant thereof, or
   b. a nucleic acid sequence comprising at least (i) SEQ ID NO: 18 (R2-MD) and SEQ ID NO: 21 (R5-MD) or (ii) SEQ ID NO: 21 (R5-MD) and at least one of SEQ ID NO: 19, SEQ ID NO: 20 (R3-MD, R4-MD) of the human of MYOD1 gene, or a functional fragment thereof, or a sequence having at least 55% sequence identity to at least SEQ ID NO: 18, SEQ ID NO: 21 or SEQ ID NO: 19, SEQ ID NO: 20, a functional variant thereof.
8. The MuSC-RC of any of paragraphs 1-7, wherein when the MuSC-RC is operatively linked to a target nucleic acid, it results in a higher expression of the target nucleic acid in skeletal muscle satellite cells as compared to the expression of a target nucleic acid operatively linked to a CK8e regulatory element having a sequence of SEQ ID NO: 306.
9. The MuSC-RC of any of paragraphs 1-8, wherein the nucleic acid sequence comprising at least a R5 and R6 RE of the PAX7 gene, comprises SEQ ID NO: 15 (R15-Px7) or SEQ ID NO: 16 (R16-Px7), or a sequence having at least 85% sequence identity to at least SEQ ID NO: 15 or SEQ ID NO: 16.
10. The MuSC-RC of any of paragraphs 1-8, wherein the nucleic acid sequence comprising at least a R5 and R6 RE of the PAX7 gene, further comprises any one or more of:
   a. a nucleic acid sequence comprising at least SEQ ID NO: 1 (R1-Px7) or a sequence having at least 85% sequence identity to SEQ ID NO: 1,
   b. a nucleic acid sequence comprising at least SEQ ID NO: 2 (R2-Px7) or a sequence having at least 85% sequence identity to SEQ ID NO: 2,
   c. a nucleic acid sequence comprising at least SEQ ID NO: 3 (R3-Px7), or a sequence having at least 85% sequence identity to SEQ ID NO: 3,
   d. a nucleic acid sequence comprising at least SEQ ID NO: 4 (R4-Px7), or a sequence having at least 85% sequence identity to SEQ ID NO: 4,
   e. a nucleic acid sequence comprising at least SEQ ID NO: 7 (R7-Px7), or a sequence having at least 85% sequence identity to SEQ ID NO: 7,
   f. a nucleic acid sequence comprising at least SEQ ID NO: 8 (R8-Px7), or a sequence having at least 85% sequence identity to SEQ ID NO: 8,
   g. a nucleic acid sequence comprising at least SEQ ID NO: 9 (R9-Px7), or a sequence having at least 85% sequence identity to SEQ ID NO: 9,
   h. a nucleic acid sequence comprising at least SEQ ID NO: 10 (R10-Px7), or a sequence having at least 85% sequence identity to SEQ ID NO: 10,
   i. a nucleic acid sequence comprising at least SEQ ID NO: 11 (R11-Px7), or a sequence having at least 85% sequence identity to SEQ ID NO: 11,
   j. a nucleic acid sequence comprising at least SEQ ID NO: 12 (R12-Px7), or a sequence having at least 85% sequence identity to SEQ ID NO: 12,
   k. a nucleic acid sequence comprising at least SEQ ID NO: 13 (R13-Px7), or a sequence having at least 85% sequence identity to SEQ ID NO: 13, or
   l. a nucleic acid sequence comprising at least SEQ ID NO: 14 (R14-Px7), or a sequence having at least 85% sequence identity to SEQ ID NO: 7.
11. The MuSC-RC of paragraphs 1-10, wherein the nucleic acid sequence comprising at least a R5 and R6 RE of the PAX7 gene comprises, or consists essentially of, a nucleic acid sequence comprising at least SEQ ID NOs: 4-7 (R7-Px7, R6-Px7, R5-Px7, R4-Px7), or a sequence having at least 85% sequence identity to SEQ ID NOs: 4-7.
12. The MuSC-RC of paragraph 7, wherein the nucleic acid sequence further comprises at least one RE selected from R4-Px7 (SEQ ID NO: 4) or R7-Px7 (SEQ ID NO: 7) or a sequence having at least 95% sequence identity thereto.
13. The MuSC-RC of paragraph 12, wherein the nucleic acid sequence further comprises at least one RE selected from R1-Px7 (SEQ ID NO: 4), R2-Px7 (SEQ ID NO: 2) and R3-Px7 (SEQ ID NO: 3) or a sequence having at least 95% sequence identity thereto.
14. The MuSC-RC of paragraph 1, wherein the nucleic acid sequence comprises two REs selected from any one or more of Pax7 RE's selected from SEQ ID NO: 1-14 (R1-Px7 to R14-Px7), or a sequence having at least 85% sequence identity to SEQ ID NO: 1-14.
15. The MuSC-RC of paragraph 7, wherein the nucleic acid sequence further comprises one or more Pax7 REs selected from any one or more of: R1-Px7 (SEQ ID NO: 1), R2-Px7 (SEQ ID NO: 2), R3-Px7 (SEQ ID NO: 3) or a portion thereof.
16. The MuSC-RC of paragraph 15, wherein the nucleic acid sequence further comprises one or more Pax7 REs selected from any one or more of: R1-Px7 (SEQ ID NO: 1), R2-Px7 (SEQ ID NO: 2), R3-Px7 (SEQ ID NO: 3), or a portion thereof.
17. The MuSC-RC of any of paragraphs 1-17, wherein the nucleic acid sequence does not comprise any of: R8-Px7 to R14-Px7 (e.g., does not comprise any of SEQ ID NOS: 8, 9, 10, 11, 12, 13 or 14), or a portion thereof.
18. The MuSC-RC of paragraph 4, wherein the nucleic acid sequence comprises a R2-MD (SEQ ID NO:18) or R5-MD (SEQ ID NO: 21) RE from the human of MYOD1 gene, further comprises any one or more of:
   a. a nucleic acid sequence comprising at least SEQ ID NO: 19 (R3-MD), or a sequence having at least 85% sequence identity to SEQ ID NO: 19,
   b. a nucleic acid sequence comprising at least SEQ ID NO: 20 (R4-MD), or a sequence having at least 85% sequence identity to SEQ ID NO: 20.
19. The MuSC-RC of paragraph 18, wherein the nucleic acid sequence comprises a R2-MD (SEQ ID NO: 18) or R5-MD (SEQ ID NO: 21) RE from the human of MYOD1 gene, and further comprises a RE selected from any one or more of:
   a. a nucleic acid sequence comprising at least R2-MD (SEQ ID NO: 18), R3-MD (SEQ ID NO: 19) and R5-MD (SEQ ID NO: 21), or a portion thereof,
   b. a nucleic acid sequence comprising at least R2-MD (SEQ ID NO: 18), R4-MD (SEQ ID NO: 20) and R6-MD (SEQ ID NO: 22), or a portion thereof,
   c. a nucleic acid sequence comprising at least R2-MD (SEQ ID NO: 18), R3-MD (SEQ ID NO: 19), R4-MD (SEQ ID NO: 20), and R5-MD (SEQ ID NO: 21), or a portion thereof, or
   d. a nucleic acid sequence comprising at least R4-MD (SEQ ID NO: 20) and R5-MD (SEQ ID NO: 21), or a portion thereof.
20. The MuSC-RC of any of paragraphs 18-19, wherein the nucleic acid sequence does not comprise R6-MD (SEQ ID NO: 22) or a coding region of the human of MYOD1 gene.
21. The MuSC-RC of paragraph 3, comprising at least one RE from 3′UTR of the human of MYOD1 gene, and at least one RE from the 5′ UTR of the human PAX7 gene.
22. The MuSC-RC of paragraph 21, comprising (i) R2-MD nucleic acid sequence of the human of MYOD1 gene (SEQ ID NO: 18), and (ii) R5-Px7 ((SEQ ID NO: 5) or R6-Px7 (SEQ ID NO: 6), or both R5-Px7 (SEQ ID NO: 5) and R6-Px7 (SEQ ID NO: 6) nucleic acid sequence of the human PAX7 gene.
23. The MuSC-RC of paragraph 22, wherein the nucleic acid sequence is selected from:
   a. R2-MD (SEQ ID NO: 18) of the human of MYOD1 gene and R15-Px7 (SEQ ID NO: 15) of the human PAX7 gene, or
   b. R2-MD (SEQ ID NO: 18) of the human of MYOD1 gene and R16-Px7 (SEQ ID NO: 16) of the human PAX7 gene.
24. The MuSC-RC of any of paragraphs 1-23, wherein the MuSC-RC has a maximal length of 1500 nucleotides.
25. A nucleic acid construct comprising the MuSC-RC, operatively linked to a target nucleic acid sequence, wherein the MuSC-RC is defined according to paragraphs 1-24.
26. The nucleic acid construct of paragraph 25, wherein the target nucleic acid sequence is encodes a nuclease.
27. The nucleic acid construct of paragraph 26, wherein the nuclease is a CRISPR-associated nuclease.
28. The nucleic acid construct of paragraph 27, wherein the target nucleic acid sequence is selected from any of: a miRNA, antisense nucleic acid sequence, gene or nucleic acid sequence encoding a therapeutic polypeptide.
29. The nucleic acid construct of paragraph 28, wherein the target nucleic acid sequence is associated with a neuromuscular disease or disorder.
30. The nucleic acid construct of paragraph 29, wherein expression of the target nucleic acid sequence reduces a pathological effect or symptom of a neuromuscular disease or disorder.
31. The nucleic acid construct of paragraph 30, wherein the neuromuscular disease or disorder is a muscular dystrophy selected from at least one of the myotonic muscular dystrophies (DM1 or DM2), Duchenne muscular dystrophy, Becker muscular dystrophy, the limb-girdle muscular dystrophies, the facioscapulohumeral muscular dystrophies, the congenital muscular dystrophies, oculopharyngeal muscular dystrophy, distal muscular dystrophy, the desmin-related myopathies, fukyama muscular dystrophy, the FKRP-deficiencies, and Emery-Dreifuss muscular dystrophy.
32. The nucleic acid construct of any of paragraphs 24-31, wherein the construct is present in a non-viral or viral vector.
33. The nucleic acid construct of paragraph 32, wherein the viral vector is an AAV vector.
34. The nucleic acid construct of any of paragraphs 24-33, wherein the construct further comprises one or more guide RNA (gRNA) cassettes.
35. A recombinant vector comprising a MUSC-RC of paragraph 1-24 or a nucleic acid construct of paragraph 24-34 comprising the MuSC-RC, operatively linked to a target nucleic acid sequence.
36. The recombinant vector of paragraph 35, wherein the vector is a selected from any of: AAV vector, non-viral DNA vector, close-circular DNA vectors.
37. A cell comprising a MUSC-RC of paragraph 1-24, or the vector of paragraph 35-36.
38. A method of expressing a transgene in a skeletal muscle satellite cell, the method comprising transducing a muscle tissue with a vector of paragraph 35.
39. A method of treating a subject with a neuromuscular disease or disorder, comprising administering to the subject a recombinant vector of paragraphs 35-36, or a cell of paragraph 37 to the subject with a neuromuscular disease or disorder.
40. A composition comprising the recombinant vector of paragraph 35 for the treatment of a subject with a neuromuscular disease or disorder.
41. Use of the recombinant vector of paragraph 34 for the preparation of a medicament for the treatment of a neuromuscular disease or disorder.
83. A composition comprising one or more regulatory cassette(s) for gene therapies targeting muscle stem cells as herein described.
84. Methods for administering the regulatory cassette(s) of paragraph 83, comprising gene therapy delivery.
85. Methods for treating muscular dystrophy comprising administering the composition of paragraph 83 or the method of paragraph 84 to a patient.
86. The method of paragraph 85, wherein the muscular dystrophy is Duchenne muscular dystrophy (DMD).

### XI. Certain Definitions

Nucleic Acid: A nucleic acid is a polymer of monomer units or “residues”. The monomer subunits, or residues, of the nucleic acids each contain a nitrogenous base (i.e., nucleobase) a five-carbon sugar, and a phosphate group. The identity of each residue is typically indicated herein with reference to the identity of the nucleobase (or nitrogenous base) structure of each residue. Canonical nucleobases include adenine (A), guanine (G), thymine (T), uracil (U) (in RNA instead of thymine (T) residues) and cytosine (C). However, the nucleic acids of the present disclosure can include any modified nucleobase, nucleobase analogs, and/or non-canonical nucleobase, as are well-known in the art. Modifications to the nucleic acid monomers, or residues, encompass any chemical change in the structure of the nucleic acid monomer, or residue, that results in a noncanonical subunit structure. Such chemical changes can result from, for example, epigenetic modifications (such as to genomic DNA or RNA), or damage resulting from radiation, chemical, or other means. Illustrative and nonlimiting examples of noncanonical subunits, which can result from a modification, include uracil (for DNA), 5-methylcytosine, 5-hydroxymethylcytosine, 5-formethylcytosine, 5-carboxycytosine b-glucosyl-5-hydroxymethylcytosine, 8-oxoguanine, 2-amino-adenosine, 2-amino-deoxyadenosine, 2-thiothymidine, pyrrolo-pyrimidine, 2-thiocytidine, or an abasic lesion. An abasic lesion is a location along the deoxyribose backbone but lacking a base. Known analogs of natural nucleotides hybridize to nucleic acids in a manner similar to naturally occurring nucleotides, such as peptide nucleic acids (PNAs) and phosphorothioate DNA.

The five-carbon sugar to which the nucleobases are attached can vary depending on the type of nucleic acid. For example, the sugar is deoxyribose in DNA and is ribose in RNA. In some instances herein, the nucleic acid residues can also be referred with respect to the nucleoside structure, such as adenosine, guanosine, 5-methyluridine, uridine, and cytidine. Moreover, alternative nomenclature for the nucleoside also includes indicating a “ribo” or deoxyribo” prefix before the nucleobase to infer the type of five-carbon sugar. For example, “ribocytosine” as occasionally used herein is equivalent to a cytidine residue because it indicates the presence of a ribose sugar in the RNA molecule at that residue. A nucleic acid polymer can be or comprise a deoxyribonucleotide (DNA) polymer, a ribonucleotide (RNA) polymer. The nucleic acids can also be or comprise a PNA polymer, or a combination of any of the polymer types described herein (e.g., contain residues with different sugars).

Peptide: As used herein, the term “peptide” refers to natural biological or artificially manufactured short chains of amino acid monomers linked by peptide (amide) bonds. As used herein, a peptide has at least 2 amino acid repeating units.

Polypeptide/Protein: As used herein, the term “polypeptide” or “protein” refers to a polymer in which the monomers are amino acid residues that are joined together through amide bonds. When the amino acids are alpha-amino acids, either the L-optical isomer or the D-optical isomer can be used, the L-isomers being preferred. The term polypeptide or protein as used herein encompasses any amino acid sequence and includes modified sequences such as glycoproteins. The term polypeptide is specifically intended to cover naturally occurring proteins, as well as those that are recombinantly or synthetically produced.

Protein: As used herein, the term “protein” refers to any of various naturally occurring substances that consist of amino-acid residues joined by peptide bonds, contain the elements carbon, hydrogen, nitrogen, oxygen, usually sulfur, and occasionally other elements (such as phosphorus or iron), and include many essential biological compounds (such as enzymes, hormones, or antibodies).

Tissue: As used herein, the term “tissue” refers to an aggregate of similar cells and cell products forming a definite kind of structural material with a specific function, in a multicellular organism.

As used herein, an “enhancer” or an “enhancer element” is a cis-acting sequence that increases the level of transcription associated with a promoter and can function in either orientation relative to the promoter and the coding sequence that is to be transcribed and can be located upstream or downstream relative to the promoter or the coding sequence to be transcribed. There are art-recognized methods and techniques for measuring function(s) of enhancer element sequences. Enhancers disclosed herein allow for selective gene expression within muscle cells or muscle stem cells.

As used herein, the phrase “transgene” refers to an exogenous nucleic acid sequence. In one example, a transgene is a gene encoding an industrially or pharmaceutically useful compound, or a gene encoding a desirable trait or function. In yet another example, the transgene encodes useful nucleic acid such as an antisense nucleic acid or RNA interference (RNAi) molecule, wherein expression of the antisense or RNAi nucleic acid sequence or the like inhibits expression of a target nucleic acid sequence. In some embodiments, a transgene encodes a therapeutic product, e.g. a protein, polypeptide or peptide.

The term “vector” is well known in the art, and as used herein refers to a nucleic acid molecule, e.g. double-stranded DNA, which may have inserted into it a nucleic acid sequence according to the present invention. A vector is suitably used to transport an inserted nucleic acid molecule into a suitable host cell.

A vector typically contains all of the necessary elements that permit transcribing the insert nucleic acid molecule, and, where applicable, translating the transcript into a polypeptide. A vector can contain the necessary elements such that, once the vector is in a host cell, the vector can replicate independently of, or coincidental with, the host chromosomal DNA; several copies of the vector and its inserted nucleic acid molecule may be generated. Vectors of the present invention can be episomal vectors (i.e., that do not integrate into the genome of a host cell), or can be vectors that integrate into the host cell genome. This definition includes both non-viral and viral vectors. Non-viral vectors include but are not limited to plasmid vectors (e.g. pMA-RQ, pUC vectors, bluescript vectors (pBS) and pBR322 or derivatives thereof that are devoid of bacterial sequences (minicircles)) transposons-based vectors (e.g. PiggyBac (PB) vectors or Sleeping Beauty (SB) vectors), etc. Larger vectors such as artificial chromosomes (bacteria (BAC), yeast (YAC), or human (HAC)) may be used to accommodate larger inserts. Viral vectors are derived from viruses and include but are not limited to retroviral, lentiviral, adeno-associated viral (AAV), adenoviral, herpes viral, hepatitis viral vectors or the like. Typically, but not necessarily, viral vectors are replication-deficient as they have lost the ability to propagate in a given cell since viral genes essential for replication have been eliminated from the viral vector. However, some viral vectors can also be adapted to replicate specifically in a given cell, such as e.g. a muscle cell. Virosomes are a non-limiting example of a vector that comprises both viral and non-viral elements, in particular they combine liposomes with an inactivated HIV or influenza virus (Yamada et al., 2003). Another example encompasses viral vectors mixed with cationic lipids.

The term “AAV vector” as used herein is well known in the art, and generally refers to an AAV vector nucleic acid including various nucleic acid sequences. An AAV vector as used herein typically comprise a heterologous nucleic acid sequence not of AAV origin as part of the vector. This heterologous nucleic acid sequence typically comprises a promoter as disclosed herein as well as other sequences of interest for the genetic transformation of a cell. In general, the heterologous nucleic acid sequence is flanked by at least one, and generally by two AAV inverted terminal repeat sequences (ITRs). An “AAV virion” or “AAV virus” or “AAV viral particle” or “AAV vector particle” refers to a viral particle composed of at least one AAV capsid polypeptide (including both variant AAV capsid polypeptides and non-variant parent capsid polypeptides) and an encapsidated polynucleotide AAV vector. If the particle comprises a heterologous nucleic acid (i.e. a polynucleotide other than a wild-type AAV genome, such as a transgene to be delivered to a mammalian cell), it can be referred to as an “AAV vector particle” or simply an “AAV vector”. Thus, production of an AAV virion or AAV particle necessarily includes production of AAV vector as such a vector is contained within an AAV virion or AAV particle.

The terms “operably linked”, “operably connected” or equivalent expressions as used herein refer to the arrangement of various nucleic acid elements relative to each other such that the elements are functionally connected and are able to interact with each other in the manner intended. Such elements may include, without limitation, a promoter, a regulatory element (RE) (e.g. enhancer or other regulatory element), a promoter element, a polyadenylation sequence, one or more introns and/or exons, and a coding sequence of a gene of interest to be expressed. The nucleic acid sequence elements, when properly oriented or operably linked, act together to modulate the activity of one another, and ultimately may affect the level of expression of an expression product. By modulate is meant increasing, decreasing, or maintaining the level of activity of a particular element. The position of each element relative to other elements may be expressed in terms of the 5′ terminus and the 3′ terminus of each element or their position upstream or downstream of another element or position (such as a promoter element), and the distance between any particular elements may be referenced by the number of intervening nucleotides, or base pairs, between the elements. As understood by the skilled person, operably linked implies functional activity, and is not necessarily related to a natural positional link. Indeed, when used in nucleic acid expression cassettes, a MuSC-RC as disclosed herein will typically be located immediately upstream of an initiation start codon (e.g., ATG or other start codon), or in some embodiments, a promoter element (although this is generally the case, it should definitely not be interpreted as a limitation or exclusion of positions within the nucleic acid expression cassette), but this need not be the case in vivo, e.g., a MuSC-RC as disclosed herein can be located downstream of a transgene, where the MuSC-RC is able to function in the same way to when it is located upstream of the initiation start codon. Hence, according to a specific embodiment, the MuSC-RC as disclosed herein can be position-independent.

A “spacer sequence” or “spacer” as used herein is a nucleic acid sequence that separates two functional regulatory elements (e.g. REs, etc.). It can have essentially any sequence, provided it does not prevent the functional RE nucleic acid sequence from functioning as desired (e.g. this could happen if it includes a silencer sequence, prevents binding of the desired transcription factor, or suchlike). Typically, a spacer is non-functional, as in it is present only to space adjacent functional nucleic acid sequences from one another. In some embodiments, spacers may have a length of 75, 50, 40, 30, 30 or 10 nucleotides or fewer.

The term “pharmaceutically acceptable” as used herein is consistent with the art and means compatible with the other ingredients of the pharmaceutical composition and not deleterious to the recipient thereof.

Therapeutic Agent: As used herein, the term “therapeutic agent” refers to a substance capable of producing a therapeutic effect in a disorder or disease state.

Therapeutically Effective Amount: As used herein, the phrase “therapeutically effective amount” refers to the amount of a therapeutic agent (i.e., vector, or cell comprising a MuSC-RC nucleic acid sequence) that elicits the biological or medicinal response that is being sought in a tissue, system, animal, individual or human by a researcher, veterinarian, medical doctor or other clinician, which includes one or more of the following:
a) preventing the disease; for example, preventing a disease, condition or disorder in an individual who may be predisposed to the disease, condition or disorder but does not yet experience or display the pathology or symptomatology of the disease;
b) inhibiting the disease; for example, inhibiting a disease, condition or disorder in an individual who is experiencing or displaying the pathology or symptomatology of the disease, condition or disorder; and
c) ameliorating the disease; for example, ameliorating a disease, condition or disorder in an individual who is experiencing or displaying the pathology or symptomatology of the disease, condition or disorder (i.e., reversing the pathology and/or symptomatology) such as decreasing the severity of disease.

In some embodiments, the term “therapeutically effective amount” and like phrases also means an appropriate dose of a vector or a cell comprising a MuSC-RC as disclosed herein to a subject that provides the desired specific effect, e.g. to transduce a muscle stem cell (e.g., muscle satellite cells) to express a transgene operatively linked to the MuSC-RC in the muscle stem cell (e.g., muscle satellite cell). The therapeutically effective amount may vary based on the route of administration and dosage form, the age and weight of the subject, and/or the disease or condition being treated.

A “small interfering RNA” or “short interfering RNA” or “siRNA” or “RNA interference molecule” is an RNA duplex of nucleotides targeted to a gene interest (a “target gene”). An “RNA duplex” refers to the structure formed by the complementary pairing between two RNA strands or two regions of an RNA molecule. An RNA interference molecule is “targeted” to a gene via complementarity to a portion of the target gene transcript; the nucleotide sequence of the duplex portion of the siRNA includes sequence complementary to a nucleotide sequence of the targeted gene transcript. In some embodiments, the length of the duplex of siRNAs is less than 30 nucleotides. In some embodiments, the duplex can be 29, 28, 27, 26, 25, 24, 23, 22, 21, 20, 19, 18, 17, 16, 15, 14, 13, 12, 11 or 10 nucleotides in length. In some embodiments, the length of the duplex is 19-25 nucleotides in length. The RNA duplex portion of the siRNA can be part of a hairpin structure. In addition to the duplex portion, the hairpin structure may contain a loop portion positioned between the two sequences forming the duplex. The loop can vary in length. In some embodiments the loop is 5, 6, 7, 8, 9, 10, 11, 12 or 13 nucleotides in length. The hairpin structure can also contain 3′ or 5′ overhang portions. In some embodiments, the overhang is a 3′ or a 5′ overhang 0, 1, 2, 3, 4 or 5 nucleotides in length. RNA interference molecules can promote cleavage of targeted transcripts through mechanisms involving formation of an RNA-Induced Silencing Complex, or RISC.

The terms “treatment” or “treating” refer to reducing, ameliorating or eliminating one or more signs, symptoms, or effects of a disease or condition. “Treatment,” as used herein thus includes any treatment of a disease in a mammal, particularly in a human, and includes: (a) preventing the disease from occurring in a subject predisposed to the disease or at risk of acquiring the disease but has not yet been diagnosed as having it; (b) inhibiting the disease, i.e., arresting or slowing its development; and (c) relieving the disease, i.e., causing regression of the disease or symptoms.

As used herein, a “neuromotor disorder” is a developmental or acquired disorder that typically affects movement/gross motor ability, posture, and fine motor ability. The disorder is caused by damage to the central nervous system. This could be due to problems with development or injury to the developing motor pathways in the cortex, basal ganglia, thalamus, cerebellum, brainstem, spinal cord, or peripheral nerve. The most common neuromotor disorders in childhood include cerebral palsy, muscular dystrophy, and spina bifida. The most common neuromotor disorders in adults include stroke, multiple sclerosis, Parkinson's disease and traumatic injury. The impairment may be static (not getting worse) or progressive.

In some embodiments, the neuromuscular or neuromotor disorder is spasticity. As used herein, “spasticity” refers to a condition in which certain muscles are continuously or abnormally contracted. This contraction causes stiffness or tightness of the muscles and can interfere with normal movement of face, limbs, trunk, and/or sphincters, leading to deficits in, for example, speech, gait, and/or bladder and bowel function. Spasticity is a condition that occurs in widespread disorders of the CNS that affect brain and/or spinal cord function, including, for example traumatic injury to brain or spinal cord, multiple sclerosis, cerebral palsy, stroke, or other conditions. Despite the underlying condition, spasticity develops when the properties of motor neurons change in response to the condition, and over-produce electrical impulses, leading to excessive muscle contraction. The damage causes a change in the balance of signals between the nervous system and the muscles, leading to increased excitability in muscles. Spasticity is found in conditions where the brain and/or spinal cord are damaged or fail to develop normally; these include cerebral palsy, multiple sclerosis, spinal cord injury, and acquired brain injury including stroke.

The term “muscle” means a structure, which is composed of myoblasts, myotubes, myofibers, stem cells that could produce myoblasts, and proteins that support those structures. Such muscle includes skeletal, cardiac, and smooth muscles.

The term “muscular health” refers to the condition of muscle wherein a subject with a deficit in that condition finds any obvious inconvenience to carry out daily life.

The term “muscle injury” refers to the condition that muscle does not function normally. The injury could be caused by excessive impact to a muscle where muscle fibers compressed in this manner can become irritated and even torn, caused when a muscle is stretched beyond its capacity and caused when intense and rapid contraction is demanded of a muscle.

The terms “muscle atrophy” and “muscle loss” refer to a condition which is caused by disuse of muscles, e.g. a lack of physical activity, or by a degenerative muscle condition. For example, a subject under medical conditions that limit their movement can lose muscle tone and develop atrophy, as can a subject with a degenerative muscle condition.

The term “muscle strength” means the amount of the force that muscle can produce with maximal efforts.

The term “cancer-associated cachexia” and “infection-induced cachexia” mean an ongoing loss of skeletal muscle mass that cannot be reversed by conventional nutritional support and leads to progressive functional impairment. Cachexia caused by cancer refers “cancer-associated cachexia,” and cachexia induced by infection is defined as “infection-induced cachexia”.

The term “aging” means the physiological process which associates a progressive functional decline, or a gradual deterioration of physiological function with age.

The term “cardiovascular disease” refers to disease of the circulatory system including the heart and blood vessels. There are four main types of cardiovascular disease: coronary heart disease, stroke, peripheral arterial disease, and aortic disease.

The term “regeneration” means the repair of cells, tissues, or organs. In the present invention, the term regeneration refers to the repair of myoblasts, myofibers, and muscular environment, which could provide an optimal environment to generate myofibers.

The “administration” of an agent to a subject includes any route of introducing or delivering to a subject the agent to perform its intended function. Administration can be carried out by any suitable route, including orally, intranasally, intraocularly, ophthalmically, parenterally (intravenously, intramuscularly, intraperitoneally, or subcutaneously), or topically. Administration includes self-administration and the administration by another. Intramuscular administration is of particular interest in the present invention.

The terms “individual,” “subject,” and “patient” are used interchangeably, and refer to any individual subject with a disease or condition in need of treatment. For the purposes of the present disclosure, the subject may be a primate, preferably a human, or another mammal, such as a dog, cat, horse, pig, goat, or bovine, and the like.

“% sequence identity” refers to a relationship between two or more sequences, as determined by comparing the sequences. In the art, “identity” also means the degree of sequence relatedness between protein, nucleic acid, or gene sequences as determined by the match between strings of such sequences. “Identity” (often referred to as “similarity”) can be readily calculated by known methods, including those described in: Computational Molecular Biology (Lesk, A. M., ed.) Oxford University Press, NY (1988); Biocomputing: Informatics and Genome Projects (Smith, D. W., ed.) Academic Press, NY (1994); Computer Analysis of Sequence Data, Part I (Griffin, A. M., and Griffin, H. G., eds.) Humana Press, NJ (1994); Sequence Analysis in Molecular Biology (Von Heijne, G., ed.) Academic Press (1987); and Sequence Analysis Primer (Gribskov, M. and Devereux, J., eds.) Oxford University Press, NY (1992). Preferred methods to determine identity are designed to give the best match between the sequences tested. Methods to determine identity and similarity are codified in publicly available computer programs. Sequence alignments and percent identity calculations may be performed using the Megalign program of the LASERGENE bioinformatics computing suite (DNASTAR, Inc., Madison, Wisconsin). Multiple alignment of the sequences can also be performed using the Clustal method of alignment (Higgins and Sharp CABIOS, 5, 151-153 (1989) with default parameters (GAP PENALTY=10, GAP LENGTH PENALTY=10). Relevant programs also include the GCG suite of programs (Wisconsin Package Version 9.0, Genetics Computer Group (GCG), Madison, Wisconsin); BLASTP, BLASTN, BLASTX (Altschul, et al., J. Mol. Biol. 215:403-410 (1990); DNASTAR (DNASTAR, Inc., Madison, Wisconsin); and the FASTA program incorporating the Smith-Waterman algorithm (Pearson, Comput. Methods Genome Res., [Proc. Int. Symp.](1994), Meeting Date 1992, 111-20. Editor(s): Suhai, Sandor. Publisher: Plenum, New York, N.Y. Within the context of this disclosure it will be understood that where sequence analysis software is used for analysis, the results of the analysis are based on the “default values” of the program referenced. As used herein “default values” will mean any set of values or parameters, which originally load with the software when first initialized.

Variants of nucleic acid sequences also include nucleic acid molecules that hybridize under intracellular conditions or for testing requirements, under stringent hybridization conditions to a sequence disclosed herein and provide the same function as such reference sequence. Exemplary stringent hybridization conditions for testing are well known to a person of ordinary skill in the art, and include an overnight incubation at 42° C. in a solution including 50% formamide, 5×SSC (750 mM NaCl, 75 mM trisodium citrate), 50 mM sodium phosphate (pH 7.6), 5×Denhardt's solution, 10% dextran sulfate, and 20 μg/ml denatured, sheared salmon sperm DNA, followed by washing the filters in 0.1×SSC at 50° C. Changes in the stringency of hybridization and signal detection are primarily accomplished through the manipulation of formamide concentration (lower percentages of formamide result in lowered stringency); salt conditions, or temperature. For example, moderately high stringency conditions include an overnight incubation at 37° C. in a solution including 6×SSPE (20×SSPE=3M NaCl; 0.2M NaH2PO4; 0.02M EDTA, pH 7.4), 0.5% SDS, 30% formamide, 100 μg/ml salmon sperm blocking DNA; followed by washes at 50° C. with 1×SSPE, 0.1% SDS. In addition, to achieve even lower stringency, washes performed following stringent hybridization can be done at higher salt concentrations (e.g. 5×SSC). Variations in the above conditions may be accomplished through the inclusion and/or substitution of alternate blocking reagents used to suppress background in hybridization experiments. Typical blocking reagents include Denhardt's reagent, BLOTTO, heparin, denatured salmon sperm DNA, and commercially available proprietary formulations. The inclusion of specific blocking reagents may require modification of the hybridization conditions described above, due to problems with compatibility.

As used herein and unless otherwise indicated, the terms “a” and “an” are taken to mean “one”, “at least one” or “one or more”. Unless otherwise required by context, singular terms used herein shall include pluralities and plural terms shall include the singular.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”. Words using the singular or plural number also include the plural and singular number, respectively. Additionally, the words “herein,” “above,” and “below” and words of similar import, when used in this application, shall refer to this application as a whole and not to any particular portions of the application.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

The complete disclosure of all patents, patent applications, and publications, and electronically available material cited herein are incorporated by reference in their entirety. Supplementary materials referenced in publications (such as supplementary tables, supplementary figures, supplementary materials and methods, and/or supplementary experimental data) are likewise incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

The description of embodiments of the disclosure is not intended to be exhaustive or to limit the disclosure to the precise form disclosed. While the specific embodiments of, and examples for, the disclosure are described herein for illustrative purposes, various equivalent modifications are possible within the scope of the disclosure.

Specific elements of any foregoing embodiments can be combined or substituted for elements in other embodiments. Moreover, the inclusion of specific elements in at least some of these embodiments may be optional, wherein further embodiments may include one or more embodiments that specifically exclude one or more of these specific elements. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

All headings are for the convenience of the reader and should not be used to limit the meaning of the text that follows the heading, unless so specified.

All of the references cited herein are incorporated by reference. Aspects of the disclosure can be modified, if necessary, to employ the systems, functions, and concepts of the above references and application to provide yet further embodiments of the disclosure. These and other changes can be made to the disclosure in light of the detailed description.

It will be appreciated that, although specific embodiments of the invention have been described herein for purposes of illustration, various modifications may be made without deviating from the spirit and scope of the invention. Accordingly, the invention is not limited except as by the claims.

### EXAMPLES

Examples and additional description and details of MuSC-RCs for specific expression of genes in muscle stem cells are described in the following.

### Example 1

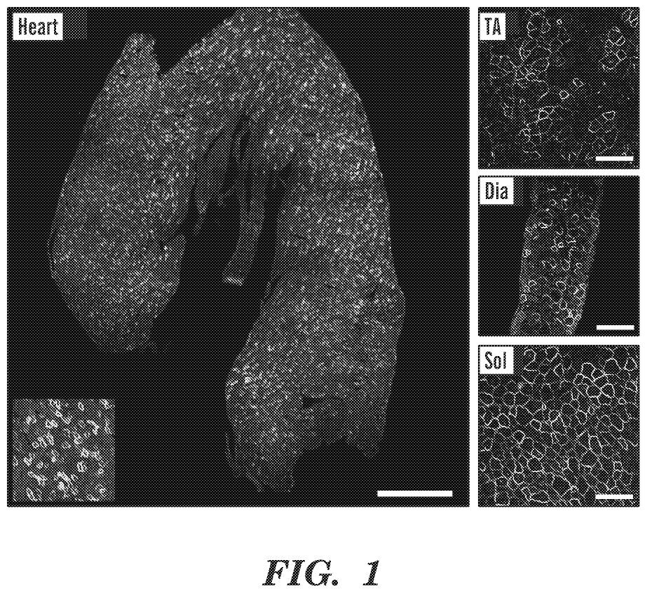

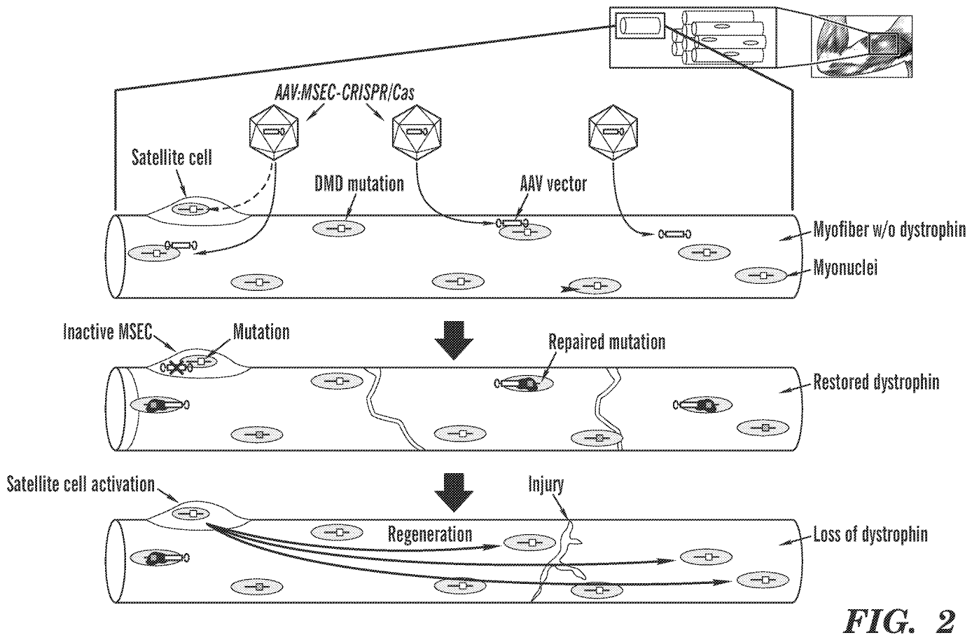

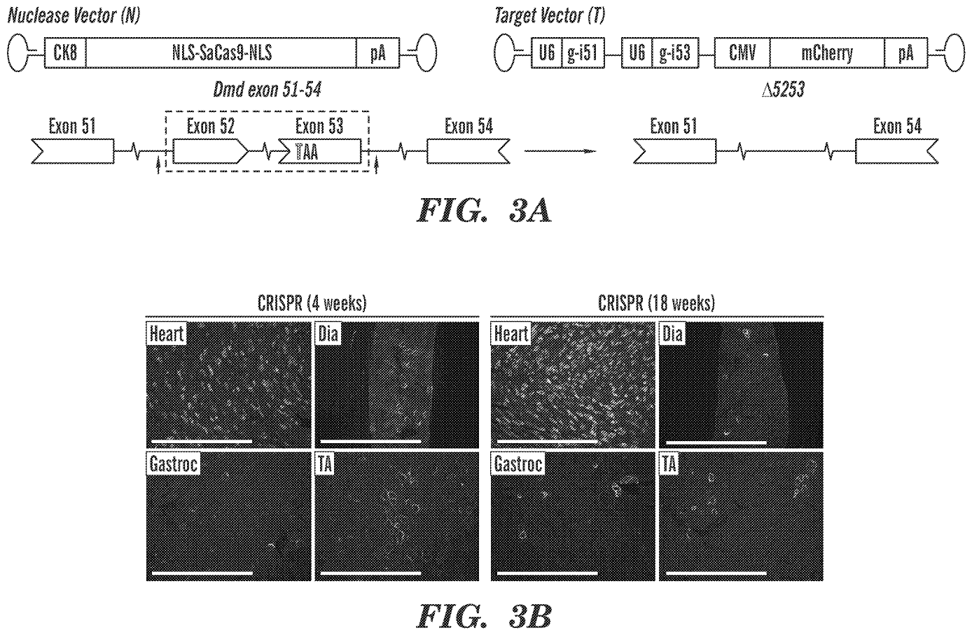

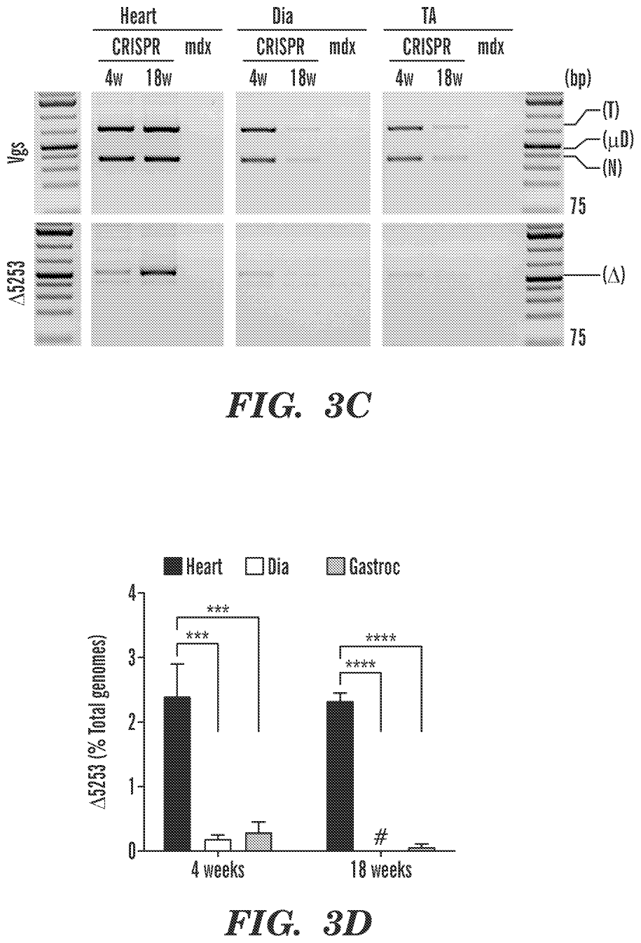

To reduce the risk of detrimental off-target effects of gene editing, the inventors previously demonstrated significant dystrophin gene correction specifically in post-mitotic muscle tissue through the use of a muscle-specific expression cassette (MSEC), (FIG. 1).^{1,2} While post-mitotic muscle-specific RCs have been reported^{11,14-16}, these RCs display negligible activities in myoblasts or muscle stem cells or satellite cells.^{2,11} Accordingly, without muscle stem cells being edited, dystrophin-corrected myonuclei (and hence dystrophin expression) can be lost over time due to skeletal muscle turnover as a result of normal maintenance or trauma (FIG. 2). The inventors demonstrate the loss of expression in post-mitotic muscle cells following systemic editing in young dystrophic mice, where gene-editing occurred during a time period marked by rapid muscle damage and regeneration, and where the absence of muscle stem cell editing coupled with a relatively low rate of editing caused loss of dystrophin-correction in skeletal-muscle, but not cardiac muscle (which does not undergo turnover) (FIG. 3A-3D).³ While the inventors demonstrate that co-delivery of microdystrophin mitigates this rapid loss of dystrophin-correction in skeletal muscle (FIG. 4D),³ this benefit may not be permanent; as some level of muscle regeneration is still to be expected over a lifetime due to for example trauma, exercise or normal muscle maintenance.

In addition to the issues of the loss of expression due to skeletal muscle turn over, there are a limited number of promoters or regulator constructs that are compatible with AAV vectors. Importantly, no AAV-size-compatible regulatory cassettes with specific activity in SCs exist (total packaging capacity of AAV is <5000 base pairs (bp)), highlighting a strong need for their development. Temporal control of editing is also attractive from a safety perspective,¹⁷ but currently available systems are either inefficient or require the continued expression of immunogenic gene products (i.e., Cre-recombinase or Tet-activators).

Accordingly, the inventors have generated synthetic promoters that specifically target muscle stem cells. The MuSC-RC disclosed herein target pools of transiently amplifying activated muscle stem cells (SCs), or myogenic progenitors. This generates dystrophin-corrected cells that would lose the episomal AAV vectors that produce potentially immunogenic editing components via cell division, drastically reducing the risk of losing edited daughter cells due to immune rejection or genotoxicity. Resulting daughter cells may then either fuse into mature dystrophin-expressing myofibers or return to quiescence to form a reserve pool of corrected SCs for future regenerative needs.

Based on the yet unmet ability to precisely target muscle stem cells, the inventors have developed RCs that permit efficient, specific and transient expression of delivered genes in quiescent and/or activated muscle SCs.

### Example 2

Design and in vitro testing of MuSC-RCs: MuSC-RC candidates were assembled by identifying and modifying conserved regulatory regions, (also referred to herein as regulatory elements or RE's) of the paired-box transcription factor Pax7 (a marker of muscle stem cells in vivo),¹⁸ and the muscle-specific transcription factor MyoD. While Pax7 expression is considered a bona fide marker of “true” quiescent satellite cells, expression of MyoD signifies activation of SCs in response to injury and is associated with rapid proliferation/amplification of myogenic stem- and progenitors that later fuse to repair injured muscle fibers. The combination of gene regulatory elements from both SC transcription factors allows for prospective targeting of both quiescent-(Pax7⁺/MyoD⁻) and/or activated-(Pax7T/MyoD⁺) SCs (FIG. 5).¹⁹

While key domains that regulate expression of Pax7²⁰⁻²² and MyoD^{23,24} have been previously reported, their use is limited as their sizes vastly exceed the packaging capacity of AAV.

Accordingly, the inventors compared vertebrate sequences within these large regulatory regions (>150 kbp across each gene locus) to identify conserved and histone-marked chromatin domains likely to indicate promoter/enhancer containing essential control elements (CEs) in the human genome. Additional narrowing of the resulting cDNA size was done by selecting regions that displayed high densities of putative myogenic transcription factor binding sites, based on their respective consensus target sequences.

In silico analyses were used to generate libraries of identified key domains from both genes (FIG. 6A) that were used to assemble a range of progressively smaller synthetic MuSC-RC candidates comprised of regulatory components (i.e., regulatory elements) derived from Pax7 or MyoD separately, or in combination as chimeric Pax7/MyoD1 MuSC-RC (FIG. 6B).

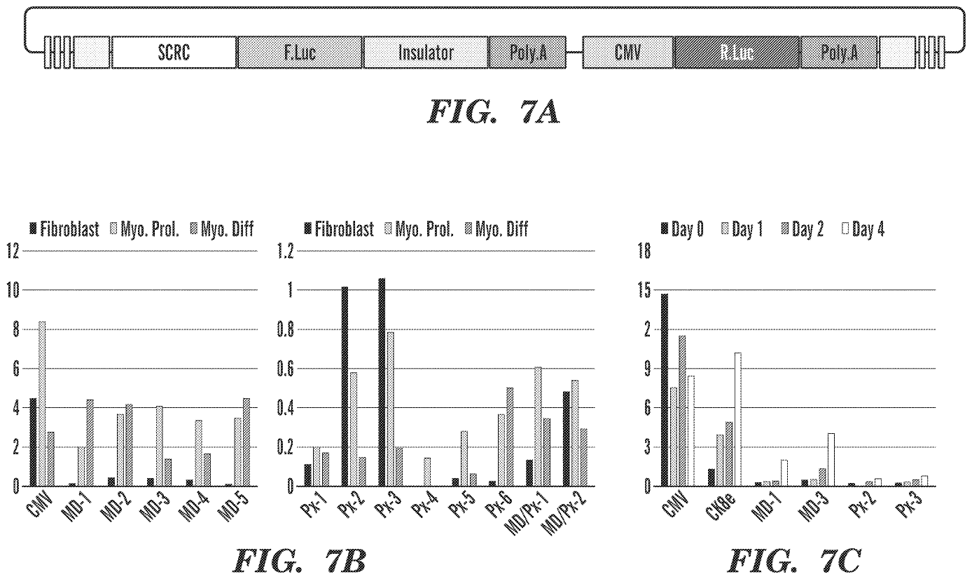

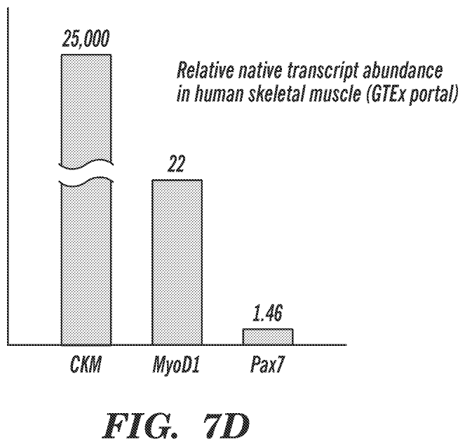

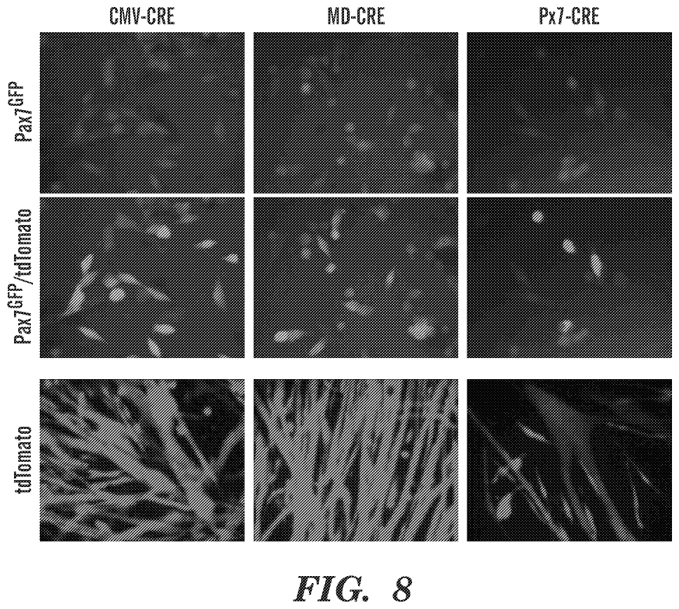

In vitro quantification of transcriptional activities of candidate MuSC-RCs was performed using a dual-luciferase plasmid transfection assay (Promega) where MuSC-RC was used drive expression of Firefly luciferase (FLuc) and normalized to CMV driving Renilla luciferase (RLuc). Both reporters were integrated into a single plasmid (see FIG. 7A) that was transfected into proliferating myogenic progenitors and differentiated myocytes derived from primary mouse SCs, or a mouse fibroblast (L6) cell line. Primary SCs were isolated from wildtype male mice hindlimb muscles using a Pronase™-based digestion protocol,²⁵ followed by Percoll density centrifugation separation of SCs from debris and non-myogenic cells.²⁶ Resulting myogenic cell populations were seeded in 2% gelatin coated culture vessels and grown under myogenic proliferation conditions in DMEM supplemented with Penstrep, 15% fetal bovine serum, 10% horse serum and 7.5 ng/mL basic FGF. Myogenic differentiation was induced by gentle washing of culture vessels with pre-warmed Saline and switching culture media to DMEM containing 2% horse serum and Insulin-Selenium-Transferrin at 4 days prior to transfection. Resulting FLuc/RLuc expression levels were measured on a SpectraMax iD3 Microplate reader at 48 hours post-transfection.

In vitro results show that MyoD1-based RCs generally exhibit higher activity levels in than Pax7-based MuSC-RCs, and approach levels achieved using CMV (FIG. 7B). Furthermore, some MyoD1-based RCs exhibit comparable activity levels in both proliferating and differentiated cultures, while still maintaining low- to negligible activity in non-myogenic fibroblasts. This seemingly promiscuous pan-myogenic activity may be beneficial for the simultaneous targeting/treatment of both activated SCs and existing post-mitotic myonuclei of dystrophic striated muscle. Despite the apparent lower in vitro activity levels, Pax7-based MuSC-RCs may yet perform significantly better in vivo as Pax7 transcription is rapidly shut off following SC isolation. Additionally, based on canonical Pax7 transcription levels in SCs, it is not clear that maximizing transcriptional activity would extrapolate into better therapeutic outcomes. In fact, closer alignment with the normal activity of SC gene expression may yield better outcomes by limiting overexpression that may lead to genotoxicity or interfere with general cellular transcription by soaking up valuable transcription factors.

Evaluating activity levels following transfection of freshly isolated SCs on days 0, 1, 2 and 4 (under proliferating conditions) with select MuSC-RCs, generally demonstrate increased activity levels at 48 hours post-transfection with time post-isolation (except for CMV) (FIG. 7C). This likely reflects a longer post-isolation growth recovery phase for myogenic progenitor cells as compared to fibroadipogenic progenitors (FAPs) that persists in low numbers following primary myogenic cell isolation without FACS enrichment, and that readily express genes regulated by CMV.

### Example 3

In Vivo activity of Candidate MuSC-RCs: To expand on the in vitro studies involving myogenic stem/progenitor cell lines, which are both time- and cost-efficient to select or eliminate RC candidates, the inventors assessed the MuSC-RC in in vivo for a more accurate representation of bona fide muscle satellite cells in vivo. Accordingly, the inventors also evaluated all MuSC-RC candidates in vivo. Here, the inventors utilized Pax7-GFP/Ail4(CAG-fl-STOP-fl-tdTomato) double-transgenic mice.^{21,27} These mice express GFP in SCs, and ubiquitous tdTomato after Cre-recombinase (CRE)-mediated excision of a premature translation termination signal,^{8,11} which enables selection of GFP+SCs and cells in which CRE was expressed (tdTomato+) via fluorescence activated cell sorting (FACS). Transfection of primary derived myogenic cell cultures from these mice with plasmids expressing CRE using CMV, MyoD- or Pax7-based RCs demonstrated sensitive detection of RC activity (FIG. 8).

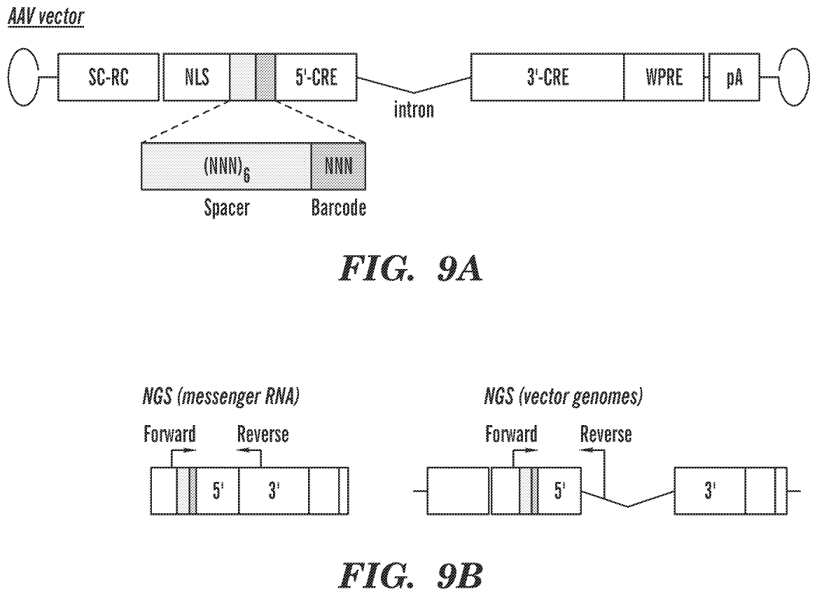

Evaluation of candidate MuSC-RCs was also performed in vivo using 4-6 week-old Pax7-GFP/Ai14 mice. MuSC-RC activities were evaluated via both direct hindlimb intramuscular (IM)- and systemic (IV) delivery after AAV delivery using myotropic AAVMYO1 vectors,²⁸ which express Cre-recombinase (CRE). To enable simultaneous head-to-head comparisons of MuSC-RC versus control CMV (constitutively active) & CK8e (predominately active in postmitotic myonuclei) RCs, the inventors delivered a pool of 15 individual AAV vectors with MuSC-RC and control RCs driving expression of a CRE cDNA separated into two parts by introduction of a human beta-globin intron.²⁹ Each vector (and hence RC) was linked to a unique “barcode” fused to the 5′-end of the first CRE cDNA half for subsequent quantification of RC activity level based on targeted next generation sequencing (NGS), (FIG. 9A). This NGS-based approach eliminates potential FACS artifacts that can arise from reliance of tdTomato expression as a primary readout, stemming from cell extrinsic tdTomato signal leading to false positive cells, as well as uptake of dissolved CRE protein once SCs are in suspension after being released from bulk muscle during digestion. Individual RC activity levels are quantified via NGS of RT-PCR amplicons generated across spliced CRE mRNA (lacking the introduced intron) and de-multiplexed based on the unique barcode associated with each individual RC. Detected mRNA levels are then normalized to the relative presence of corresponding barcoded AAV vector genomes via NGS of PCR amplicons that still carry the intron (FIG. 9B). This approach enables direct quantitative comparisons of transcriptional activities from the various RCs (based on CRE mRNA levels) in both activated and quiescent muscle stem cells (SC) versus committed progenitors, regardless of individual AAV vector transduction levels. A total vector dose of 3×10¹⁰ vg/injection (IM) and 1×10¹² mouse (IV) was split 1/15 among SC- and control RCs and administered into Pax7GFP; Ail4 mice. For IM delivery, injection-related injury ensured presence of both quiescent and activated SCs.

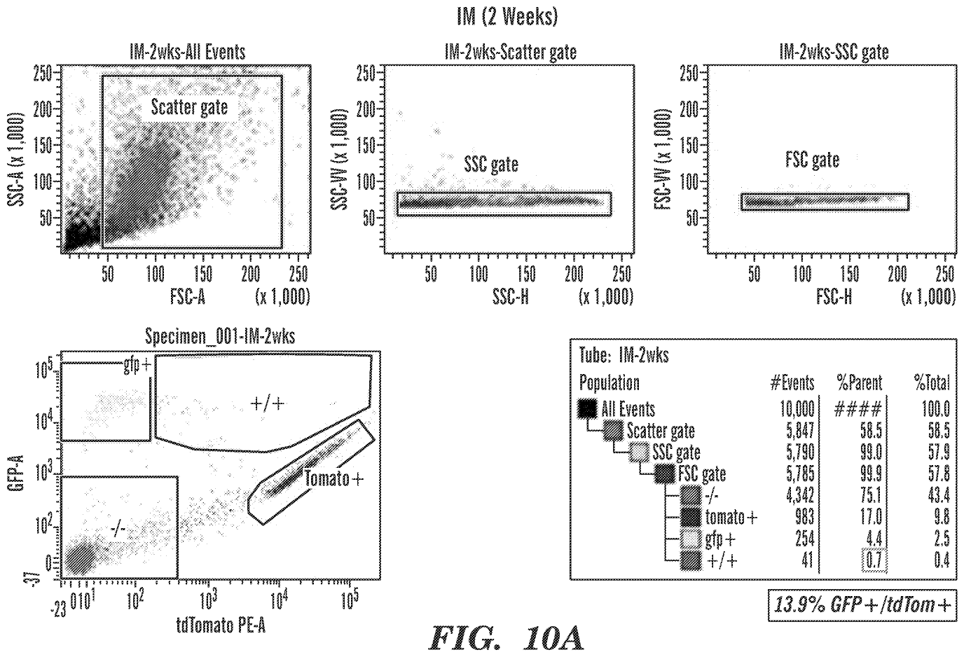

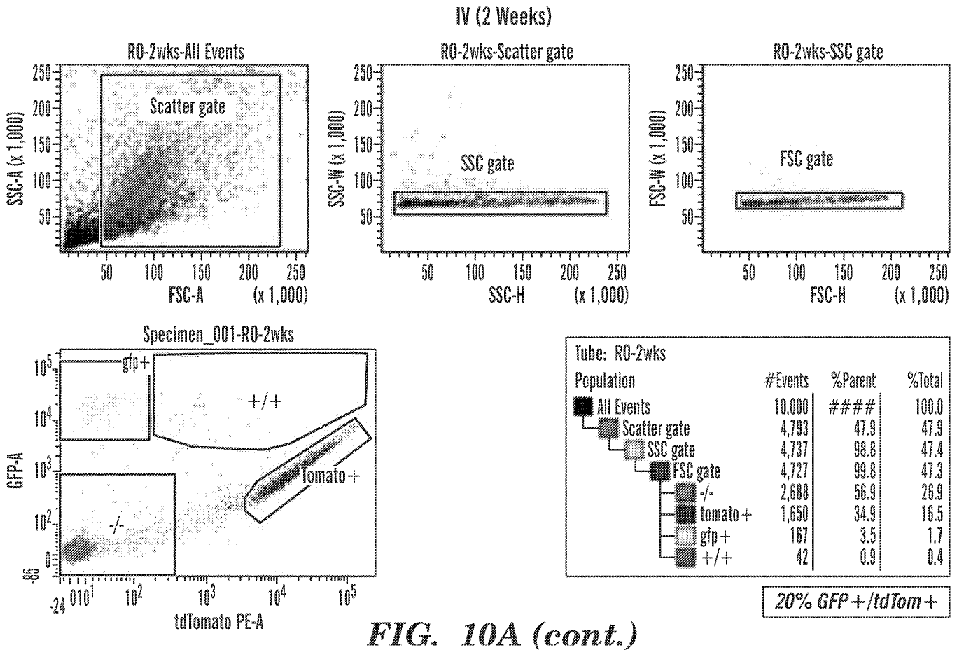

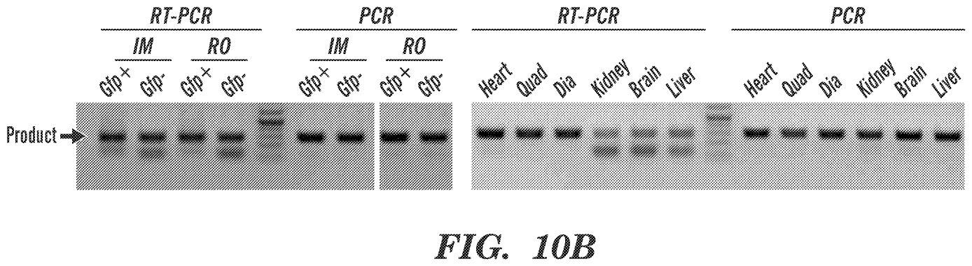

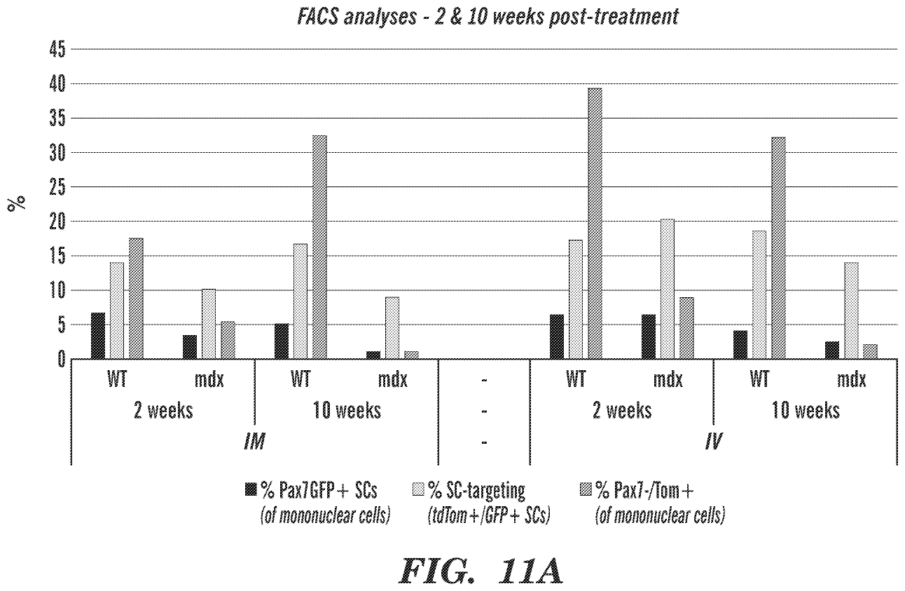

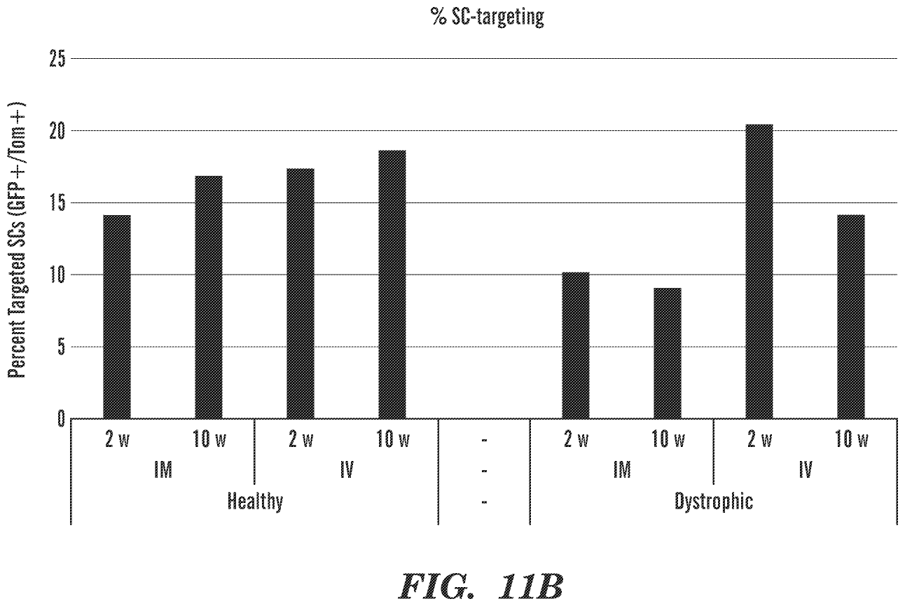

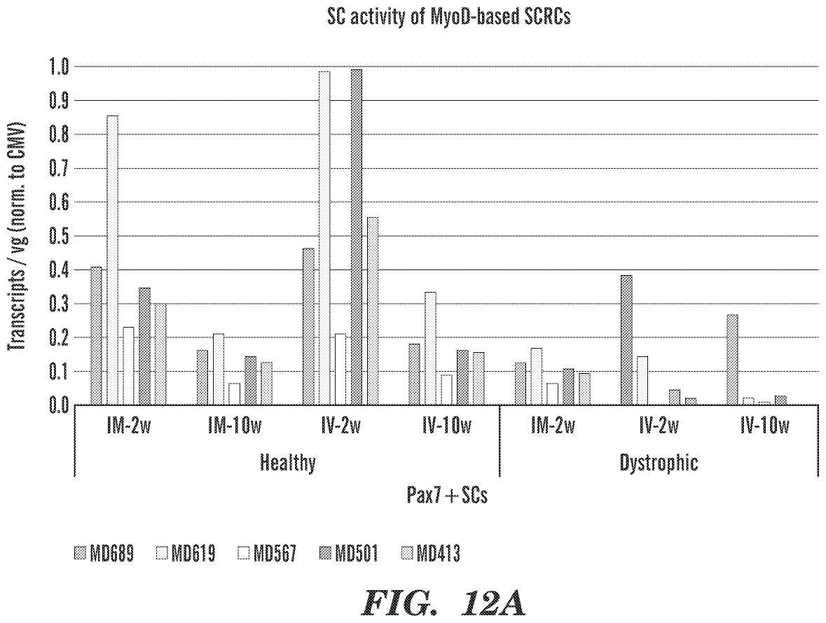

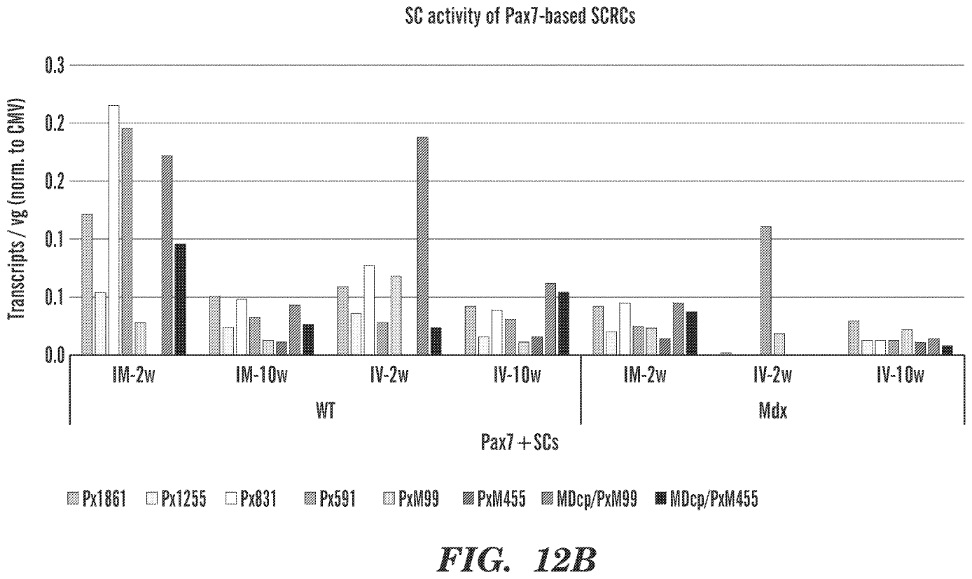

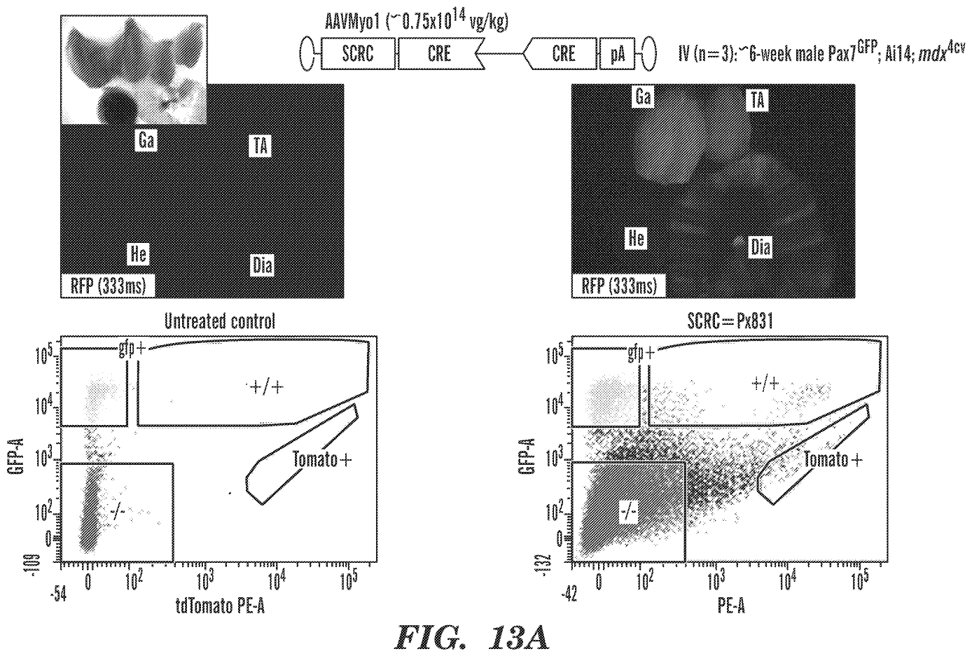

At 2 weeks post-transduction, DNA and RNA was extracted from GFP+SCs and GFP-/tdTomato+ cells isolated using FACS of single-cell suspensions from enzymatically-digested muscles (FIG. 10A), amplified and submitted for NGS (FIG. 10B).^{8,11,25}

Different from previous reports,^{8,11} we isolated SCs from bulk muscle tissue via enzymatic digestion using Pronase prior to FACS for cell-intrinsic GFP and tdTomato fluorescence.²⁵ This method, as opposed to more gentle digestion protocols for sorting cells via antigen-binding to cell surface receptors, removes the majority of ‘sticky’ cell surface receptors that may otherwise non-specifically bind tdTomato protein that is released from lysed bulk muscle and results in ‘false’ positives.^{8,11} Further validation of SC-RC activity and specificity was performed using NGS analysis of amplicons from bulk RNA and DNA isolated from quadriceps and cardiac muscles, as well as non-muscle tissue (e.g., liver, kidney and brain), (FIG. 10B, bottom right).

The inventors compared the in vitro results with the in vivo results achieved with each regulatory element (RE) derived from Pax7 or MyoD1, and surprisingly discovered that the in vitro results does not always predict the in vivo results. The comparisons are described in Table 6.

TABLE 6
Table showing the differences in transgene expression
in vitro and in vivo with Pax7 RE's and MyoD RE's.

| Pax7 RE's | | | MyoD1 RE's | | |
|---|---|---|---|---|---|
| Pax7 | | | MyoD1 | | |
| regulatory | In vitro | In vivo | regulatory | In vitro | In vivo |
| elements | effect | effects | elements | effect | effects |
| R1-R3 | Reduce activity | Reduced | R2 | Increased | Increased |
| | in proliferating | activity | | activity in | activity |
| | SCs | in SCs | | proliferative | in SC |
| | | | | cells | |
| R4 | Reduced SC | Reduced | R3 | reduced | reduced |
| | specificity | activity | | activity in | activity |
| | (elevated activity | in SCs | | proliferative | in SC |
| | in differentiated- | | | cells | |
| | or non-muscle | | | | |
| | cells) | | | | |
| R4, R5, R6 | Possible basal | | R4 | Increases | Increased |
| R15 | MuSC promoter | | | differentiation | activity |
| R16 | | | | activity | in SC |
| R7 | Increases activity | Reduced | R5 | Basal | |
| | in differentiated | activity | | promoter? | |
| | muscle cells | in SCs | | | |
| R8 | Increased all | Increased | R6 | | |
| | activity (specific | activity | | | |
| | and non- | in SC | | | |
| | specific). Could | | | | |
| | be essential RE | | | | |
| R9-R14 | reduces activity | Increased | | | |
| | in proliferating | activity | | | |
| | SC & increases | in SC | | | |
| | myogenic | | | | |
| | specificity | | | | |

While the inventors surprisingly discovered that the RE's often resulted in different transgene expression in SCs in vitro and in vivo as shown in Table 6, at MuSC-RC that contains the MyoD1 region 3 (R3-MD) showed reduced transgene activity in proliferating SCs in vitro and also reduced transgene activity in SCs in vivo; showing some agreement.

In summary, from the designed MuSC-RC-cassettes: MD689 and MD619 appear most promising. Particularly based on WT results which are easier to interpret due to steady-state muscle dynamics. Of Pax7-based RCs: Px831 and Px531 perform best. It is important to note that, as discussed herein above, SCRCs with lower apparent activities can still offer unique benefits where lesser, tuned or titrated expression is desired.

### REFERENCES

References: The references disclosed in the specification and Examples are each incorporated herein in their entirety.
1 Salva, M. Z. et al. Design of tissue-specific regulatory cassettes for high-level rAAV-mediated expression in skeletal and cardiac muscle. Mol Ther 15, 320-329, doi:10.1038/sj.mt.6300027 (2007).
2 Bengtsson, N. E. et al. Muscle-specific CRISPR/Cas9 dystrophin gene editing ameliorates pathophysiology in a mouse model for Duchenne muscular dystrophy. Nat Commun 8, 14454, doi:10.1038/ncomms14454 (2017).
3 Bengtsson, N. E., Tasfaout, H., Hauschka, S. D. & Chamberlain, J. S. Dystrophin Gene-Editing Stability Is Dependent on Dystrophin Levels in Skeletal but Not Cardiac Muscles. Mol Ther 29, 1070-1085, doi:10.1016/j.ymthe.2020.11.003 (2021).
4 Bengtsson, N. E. et al. Comparison of dystrophin expression following gene editing and gene replacement in an aged preclinical DMD animal model. Mol Ther, doi:10.1016/j.ymthe.2022.02.003 (2022).
5 Young, C. S., Pyle, A. D. & Spencer, M. J. CRISPR for Neuromuscular Disorders: Gene Editing and Beyond. Physiology 34, 341-353, doi: 10.1152/physiol.00012.2019 (2019).
6 Bengtsson, N. E., Seto, J. T., Hall, J. K., Chamberlain, J. S. & Odom, G. L. Progress and prospects of gene therapy clinical trials for the muscular dystrophies. Hum Mol Genet, doi: 10.1093/hmg/ddv420 (2015).
7 Nelson, C. E. et al. In vivo genome editing improves muscle function in a mouse model of Duchenne muscular dystrophy. Science 351, 403-407, doi:10.1126/science.aad5143 (2016).
8 Tabebordbar, M. et al. In vivo gene editing in dystrophic mouse muscle and muscle stem cells. Science 351, 407-411, doi:10.1126/science.aad5177 (2016).
9 Long, C. et al. Postnatal genome editing partially restores dystrophin expression in a mouse model of muscular dystrophy. Science 351, 400-403, doi: 10.1126/science.aad5725 (2016).
10 Duchene, B. L. et al. CRISPR-Induced Deletion with SaCas9 Restores Dystrophin Expression in Dystrophic Models In Vitro and In Vivo. Mol Ther 26, 2604-2616, doi:10.1016/j.ymthe.2018.08.010 (2018).
11 Kwon, J. B. et al. In Vivo Gene Editing of Muscle Stem Cells with Adeno-Associated Viral Vectors in a Mouse Model of Duchenne Muscular Dystrophy. Mol Ther Methods Clin Dev 19, 320-329, doi:10.1016/j.omtm.2020.09.016 (2020).
12 Goldstein, J. M. et al. In Situ Modification of Tissue Stem and Progenitor Cell Genomes. Cell reports 27, 1254-1264 e1257, doi:10.1016/j.celrep.2019.03.105 (2019).
13 Tabebordbar, M. et al. Directed evolution of a family of AAV capsid variants enabling potent muscle-directed gene delivery across species. Cell 184, 4919-4938 e4922, doi:10.1016/j.cell.2021.08.028 (2021).
14 Amoasii, L. et al. Single-cut genome editing restores dystrophin expression in a new mouse model of muscular dystrophy. Science translational medicine 9, doi:10.1126/scitranslmed.aan8081 (2017).
15 Amoasii, L. et al. Gene editing restores dystrophin expression in a canine model of Duchenne muscular dystrophy. Science 362, 86-91, doi: 10.1126/science.aaul549 (2018).
16 Mendell, J. R. et al. Assessment of Systemic Delivery of rAAVrh74.MHCK7.microdystrophin in Children With Duchenne Muscular Dystrophy: A Nonrandomized Controlled Trial. JAMA Neurol, doi:10.1001/jamaneurol.2020.1484 (2020).
17 Bengtsson, N. E. C. J. S. Enhanced Dystrophin Gene Editing and Cas9 Inactivation via AAV Re-Administration in a Mouse Model of Duchenne Muscular Dystrophy Mol Ther 30(4S1), 269-270, doi: 10.1016/j.ymthe.2022.04.017 (2022).
18 Lepper, C., Partridge, T. A. & Fan, C. M. An absolute requirement for Pax7-positive satellite cells in acute injury-induced skeletal muscle regeneration. Development 138, 3639-3646, doi:10.1242/dev.067595 (2011).
19 Zammit, P. S. et al. Pax7 and myogenic progression in skeletal muscle satellite cells. Journal of cell science 119, 1824-1832, doi: 10.1242/jcs.02908 (2006).
20 Syagailo, Y. V. et al. Structural and functional characterization of the human PAX7 5′-flanking regulatory region. Gene 294, 259-268 (2002).
21 Tichy, E. D. et al. A robust Pax7EGFP mouse that enables the visualization of dynamic behaviors of muscle stem cells. Skeletal muscle 8, 27, doi:10.1186/s13395-018-0169-7 (2018).
22 Murmann, O. V., Niggli, F. & Schafer, B. W. Cloning and characterization of the human PAX7 promoter. Biological chemistry 381, 331-335, doi:10.1515/BC.2000.043 (2000).
23 Goldhamer, D. J., Faerman, A., Shani, M. & Emerson, C. P., Jr. Regulatory elements that control the lineage-specific expression of myoD. Science 256, 538-542, doi:10.1126/science.1315077 (1992).
24 Tapscott, S. J., Lassar, A. B. & Weintraub, H. A novel myoblast enhancer element mediates MyoD transcription. Molecular and cellular biology 12, 4994-5003, doi:10.1128/mcb.12.11.4994 (1992).
25 Danoviz, M. E. & Yablonka-Reuveni, Z. Skeletal muscle satellite cells: background and methods for isolation and analysis in a primary culture system. Methods in molecular biology 798, 21-52, doi:10.1007/978-1-61779-343-1_2 (2012).
26 Yablonka-Reuveni, Z. & Nameroff, M. Skeletal muscle cell populations. Separation and partial characterization of fibroblast-like cells from embryonic tissue using density centrifugation. Histochemistry 87, 27-38, doi:10.1007/BF00518721 (1987).
27 Madisen, L. et al. A robust and high-throughput Cre reporting and characterization system for the whole mouse brain. Nature neuroscience 13, 133-140, doi:10.1038/nn.2467 (2010).
28 Weinmann, J. et al. Identification of a myotropic AAV by massively parallel in vivo evaluation of barcoded capsid variants. Nat Commun 11, 5432, doi:10.1038/s41467-020-19230-w (2020).
29 Kaczmarczyk, S. J. & Green, J. E. A single vector containing modified cre recombinase and LOX recombination sequences for inducible tissue-specific amplification of gene expression. Nucleic acids research 29, E56-56, doi:10.1093/nar/29.12.e56 (2001).

1. A muscle-stem cell specific regulatory nucleic acid cassette (MuSC-RC) for selectively regulating the expression of an operatively linked target nucleic acid in a muscle stem cell (mSC), the MuSC-RC comprising at least two regulatory elements (REs), wherein the two REs are selected from:
(i) at least two REs located in an untranslated region of the PAX7 gene,
(ii) at least two REs located in an untranslated region in the MyoD1 gene, or
(iii) at least one RE located in an untranslated region of the PAX7 gene, and at least one RE located in an untranslated region in the MyoD1 gene,
wherein the two REs are located adjacent to each other and are recombinant with respect to each other.

2. The MuSC-RC of claim 1, wherein the muscle stem cell (mSC) is a skeletal muscle satellite cell.

3. The MuSC-RC of claim 1, comprising at least one of the following:
a. a nucleic acid sequence comprising at least one RE from the untranslated region of the human PAX7 gene, or a functional fragment thereof, and
a nucleic acid sequence comprising at least one from the untranslated region of the human MYOD1 gene, or a functional fragment thereof;
b. a nucleic acid sequence comprising at least two REs from the 3′ UTR of the human PAX7 gene, or a functional fragment thereof;
c. a nucleic acid sequence comprising at least two REs from the 5′ UTR of the human MYOD1 gene, or a functional fragment thereof;
d. a nucleic acid sequence comprising at least one RE from the 5′ UTR of the human PAX7 gene, or a functional fragment thereof, and at least one regulatory element (RE) from the 5′ UTR of the human MyoD1 gene;
e. at least one RE from 3′UTR of the human MYOD1 gene, and at least one RE from the 5′ UTR of the human PAX7 gene;
f. (i) R2 nucleic acid sequence of the human MYOD1 gene, and (ii) R5 or R6, or both R5 and R6 nucleic acid sequence of the human PAX7 gene;
g. R2 of the human MYOD1 gene and R15 of the human PAX7 gene; and/or
h. R2 of the human MYOD1 gene and R16 of the human PAX7 gene.

4. (canceled)

5. The MuSC-RC of claim 3, wherein a RE from the human PAX7 gene is selected from any of: SEQ ID NO: 1-16, or a nucleic acid sequence having at least 85% sequence identity thereto; and/or a RE from the human MyoD1 gene is selected from any of: SEQ ID NO: 17-22, or a nucleic acid sequence having at least 85% sequence identity thereto.

6. (canceled)

7. The MuSC-RC of claim 1, comprising any one of:
a. a nucleic acid sequence comprising SEQ ID NO: 4 and SEQ ID NO: 5 (R4-Px7 and R5-Px7 from the human PAX7 gene), or a functional fragment thereof, or a sequence having at least 85% sequence identity to at least SEQ ID NO: 4 or SEQ ID NO:5 or a functional variant thereof;
b. a nucleic acid sequence comprising at least (i) SEQ ID NO: 18 (R2-MD) and SEQ ID NO: 21 (R5-MD) or (ii) SEQ ID NO: 21 (R5-MD) and at least one of SEQ ID NO: 19, SEQ ID NO: 20 (R3-MD, R4-MD) of the human MYOD1 gene, or a functional fragment thereof, or a sequence having at least 55% sequence identity to at least SEQ ID NO: 18, SEQ ID NO: 21 or SEQ ID NO: 19, SEQ ID NO: 20, a functional variant thereof;
c. a nucleic acid sequence comprising at least a R5 and R6 RE of the PAX7 gene, and which comprises SEQ ID NO: 15 (R15-Px7) or SEQ ID NO: 16 (R16-Px7), or a sequence having at least 85% sequence identity to at least SEQ ID NO: 15 or SEQ ID NO: 16; or
d. a nucleic acid sequence comprising two REs selected from any one or more of Pax7 REs selected from SEQ ID NO: 1-14 (R1-Px7 to R14-Px7), or a sequence having at least 85% sequence identity to one of SEQ ID NO: 1-14.

8. The MuSC-RC claim 1, wherein when the MuSC-RC is operatively linked to a target nucleic acid, it results in a higher expression of the target nucleic acid in skeletal muscle satellite cells as compared to the expression of a target nucleic acid operatively linked to a CK8e regulatory element having a sequence of SEQ ID NO: 306.

9. (canceled)

10. The MuSC-RC of claim 1, wherein the nucleic acid sequence comprises at least a R5 and R6 RE of the PAX7 gene, and further comprises any one or more of:
a. a nucleic acid sequence comprising at least SEQ ID NO: 1 (R1-Px7) or a sequence having at least 85% sequence identity to SEQ ID NO: 1,
b. a nucleic acid sequence comprising at least SEQ ID NO: 2 (R2-Px7) or a sequence having at least 85% sequence identity to SEQ ID NO: 2,
c. a nucleic acid sequence comprising at least SEQ ID NO: 3 (R3-Px7), or a sequence having at least 85% sequence identity to SEQ ID NO: 3,
d. a nucleic acid sequence comprising at least SEQ ID NO: 4 (R4-Px7), or a sequence having at least 85% sequence identity to SEQ ID NO: 4,
e. a nucleic acid sequence comprising at least SEQ ID NO: 7 (R7-Px7), or a sequence having at least 85% sequence identity to SEQ ID NO: 7,
f. a nucleic acid sequence comprising at least SEQ ID NO: 8 (R8-Px7), or a sequence having at least 85% sequence identity to SEQ ID NO: 8,
g. a nucleic acid sequence comprising at least SEQ ID NO: 9 (R9-Px7), or a sequence having at least 85% sequence identity to SEQ ID NO: 9,
h. a nucleic acid sequence comprising at least SEQ ID NO: 10 (R10-Px7), or a sequence having at least 85% sequence identity to SEQ ID NO: 10,
i. a nucleic acid sequence comprising at least SEQ ID NO: 11 (R11-Px7), or a sequence having at least 85% sequence identity to SEQ ID NO: 11,
j. a nucleic acid sequence comprising at least SEQ ID NO: 12 (R12-Px7), or a sequence having at least 85% sequence identity to SEQ ID NO: 12,
k. a nucleic acid sequence comprising at least SEQ ID NO: 13 (R13-Px7), or a sequence having at least 85% sequence identity to SEQ ID NO: 13,
l. a nucleic acid sequence comprising at least SEQ ID NO: 14 (R14-Px7), or a sequence having at least 85% sequence identity to SEQ ID NO: 7, or
m. a nucleic acid sequence comprising at least SEQ ID NOs: 4-7 (R7, R6, R5, R4), or a sequence having at least 85% sequence identity to SEQ ID NOs: 4-7.

11. (canceled)

12. The MuSC-RC of claim 7, wherein the nucleic acid sequence further comprises:
a. at least one RE selected from R4 or R7 or a sequence having at least 95% sequence identity thereto;
b. at least one RE selected from R1, R2 and R3 or a sequence having at least 95% sequence identity thereto; and/or
c. one or more Pax7 REs selected from any one or more of: R1, R2, R3 or a portion thereof.

13. (canceled)

14. (canceled)

15. (canceled)

16. (canceled)

17. The MuSC-RC of claim 1, wherein the nucleic acid sequence does not comprise any of: R8-R14, or a portion thereof; and/or wherein the nucleic acid sequence does not comprise R6 or a coding region of the human MYOD1 gene.

18. The MuSC-RC of claim 3, wherein the nucleic acid sequence comprises a R2 or R5 RE from the human MYOD1 gene, and further comprises any one or more of:
a. a nucleic acid sequence comprising at least SEQ ID NO: 19 (R3-MD), or a sequence having at least 85% sequence identity to SEQ ID NO: 19,
b. a nucleic acid sequence comprising at least SEQ ID NO: 20 (R4-MD), or a sequence having at least 85% sequence identity to SEQ ID NO: 20
c. a nucleic acid sequence comprising at least R2, R3 and R5, or a portion thereof,
d. a nucleic acid sequence comprising at least R2, R4 and R6, or a portion thereof,
e. a nucleic acid sequence comprising at least R2, R3, R4, and R5, or a portion thereof, and/or
f. a nucleic acid sequence comprising at least R4 and R5, or a portion thereof.

19. (canceled)

20. (canceled)

21. (canceled)

22. (canceled)

23. (canceled)

24. The MuSC-RC of claim 1, wherein the MuSC-RC has a maximal length of 1550 nucleotides.

25. A nucleic acid construct comprising the MuSC-RC of claim 1, operatively linked to a target nucleic acid sequence.

26. The nucleic acid construct of claim 25, wherein the target nucleic acid sequence:
a. encodes a nuclease,
b. encodes a CRISPR-associated nuclease;
c. is selected from any of: a miRNA, antisense nucleic acid sequence, gene or nucleic acid sequence encoding a therapeutic polypeptide; and/or
d. is associated with a neuromuscular disease or disorder.

27. (canceled)

28. (canceled)

29. (canceled)

30. The nucleic acid construct of claim 2926, wherein expression of the target nucleic acid sequence reduces a pathological effect or symptom of a neuromuscular disease or disorder.

31. The nucleic acid construct of claim 30, wherein the neuromuscular disease or disorder is a muscular dystrophy selected from at least one of the group consisting of: the myotonic muscular dystrophies (DM1 or DM2), Duchenne muscular dystrophy, Becker muscular dystrophy, the limb-girdle muscular dystrophies, the facioscapulohumeral muscular dystrophies, the congenital muscular dystrophies, oculopharyngeal muscular dystrophy, distal muscular dystrophy, the desmin-related myopathies, fukyama muscular dystrophy, the FKRP-deficiencies, and Emery-Dreifuss muscular dystrophy.

32. (canceled)

33. (canceled)

34. The nucleic acid construct of claim 24, wherein the construct further comprises one or more guide RNA (gRNA) cassettes.

35. A recombinant vector comprising the MuSC RC of claim 1, operatively linked to a target nucleic acid sequence; wherein the vector is selected from any of: adeno-associated virus (AAV) vector, non-viral DNA vector, or close-circular DNA vectors.

36. (canceled)

37. A cell comprising the MuSC RC of claim 1.

38. A method of expressing a target nucleic acid in a skeletal muscle satellite cell, the method comprising transducing a muscle tissue with the vector of claim 35.

39. A method of treating a subject with a neuromuscular disease or disorder, comprising administering to the subject a recombinant vector of claim 35 to the subject with a neuromuscular disease or disorder.

40. (canceled)

41. (canceled)
